SEQUENCE LISTING (5 sequences)

SEQ ID NO: 1 - DNA, 20 bp, Artificial Sequence
  Synthetic
cactctttccctacacgacg

SEQ ID NO: 2 - DNA, 20 bp, Artificial Sequence
  Synthetic
agttcagacgtgtgctcttc

SEQ ID NO: 3 - DNA, 20 bp, Artificial Sequnece
  Synthetic
cactctttccctacacgacg

SEQ ID NO: 4 - DNA, 19 bp, Artificial Sequence
  Synthetic
atgtccaggtcatagctcc

SEQ ID NO: 5 - DNA, 28 bp, Artificial Sequence
  Synthetic
caacaaacatgccatctccttctcctga

(19) United States
(12) Patent Application Publication
(10) Pub. No.: US 2023/0203568 A1
(43) Pub. Date: Jun. 29, 2023

(54) MINOR ALLELE ENRICHMENT SEQUENCING THROUGH RECOGNITION OLIGONUCLEOTIDES

(71) Applicants: The Broad Institute, Inc., Cambridge, MA (US); Dana-Farber Cancer Institute, Inc., Boston, MA (US)

(72) Inventors: Viktor A. Adalsteinsson, Cambridge, MA (US); Gregory Gydush, Cambridge, MA (US); Gerassimos Makrigiorgos, Boston, MA (US); Erica Nguyen, Cambridge, MA (US)

(73) Assignees: The Broad Institute, Inc., Cambridge, MA (US); Dana-Farber Cancer Institute, Inc., Boston, MA (US)

(21) Appl. No.: 17/792,638

(22) PCT Filed: Jan. 14, 2021
(86) PCT No.: PCT/US2021/013520

§ 371 Date: Jul. 13, 2022

Related U.S. Application Data
(60) Provisional application No. 63/124,424, filed on Dec. 11, 2020.
(60) Provisional application No. 62/961,098, filed on Jan. 14, 2020.

(51) Int. Cl. C12Q 1/683; C12Q 1/686; C12Q 1/6886; C12Q 1/6869

(52) U.S. Cl. CPC C12Q 1/683; C12Q 1/686; C12Q 1/6869; C12Q 1/6886; C12Q 2600/156

(57) ABSTRACT

The disclosure provides novel methods, compositions, and kits that combine hybrid capture using short allele-specific probes with duplex molecular” barcoding and noise modeling within each sample to afford high accuracy sequencing of rare mutations at low cost.

### RELATED APPLICATIONS

This application claims priority under 35 § U.S.C. 119(e) to U.S. Provisional Application Serial No. 62/961,098, filed Jan. 14, 2020. In addition, this application claims priority under 35 § U.S.C. 119(e) to U.S. Provisional Application Serial No. 63/124,424, filed Dec. 11, 2020. The entire contents of each of these prior applications are incorporated herein by reference.

### STATEMENT REGARDING FEDERALLY SPONSORED RESEARCH OR DEVELOPMENT

This invention was made with government support under R01 CA22187 and R03 CA217652 awarded by the National Institutes of Health. The government has certain rights in the invention.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BACKGROUND OF THE INVENTION

Mutations in DNA emerge from single cells¹, define cell populations², and establish genetic diversity³. Considering the vast genetic diversity of living organisms and the significance of mutations in disease biology⁴, there is a growing need to assay many distinct, low-abundance mutations in multiple areas of biomedicine spanning oncology⁵, obstetrics⁶, transplantation^{7,8}, infectious disease⁹, genetics ¹⁰, microbiomics¹¹, forensics¹², and beyond. Yet, the intrinsic tradeoff in breadth-versus-depth of DNA sequencing means that either few mutations can be assayed at high depth, or many mutations at low depth—not both. High depth (i.e., many reads per genomic locus) is required to accurately detect low-abundance mutations, but this severely limits breadth (i.e., number of distinct loci). This explains why, despite massive reductions in sequencing costs, it remains prohibitively expensive to test for large numbers of distinct, low-abundance mutations.

For example, duplex sequencing is one of the most accurate methods for mutation detection, with 1000-fold fewer errors than standard sequencing, however it remains prohibitively expensive due to its requirement for significantly higher number of sequence reads¹³. By requiring mutations to be present in replicate reads from both strands of each DNA duplex, many of the errors in sample preparation and sequencing can be overcome to enable reliable detection of low-abundance mutations. Yet, up to 100-fold more reads per locus are required—a challenge that is exacerbated when tracking many low-abundance mutations. Less stringent methods exist that require fewer reads, however, compromising specificity to save cost would be deeply problematic for applications that impact patient care (e.g., liquid biopsies).

There remains a significant need in the art for new approaches that significantly lower sequencing costs involved in the detection and/or tracking of large numbers of distinct, low-abundance mutations, such as in applications such as liquid biopsies for detecting minimal residual disease (MRD) after cancer treatment and the like.

### SUMMARY OF THE INVENTION

The disclosure provides new methods, compositions, and kits for detecting and/or tracking large numbers of distinct, low-abundance mutations with minimal sequencing required by enriching for low-abundance mutations prior to sequencing, e.g., duplex sequencing. The approach disclosed here—referred to as minor allele enrichment sequencing targeting rare occurrences (MAESTRO)—significantly reduces sequencing costs involved in the detection and/or tracking of large numbers of distinct, low-abundance mutations in applications, such as, but not limited to, liquid biopsies for detecting and tracking low-abundance mutations (e.g., using liquid biopsies for monitoring the presence of low-level genetic aberrations or residual genetic information related to a disorder (e.g., cancer), for example, without limitation, minimal residual disease (MRD)). In various aspects, the approach described herein combines hybrid capture using short allele-specific probes with duplex molecular barcoding and noise modeling within each sample to afford high accuracy sequencing of thousands of rare mutations at low cost.

In one aspect, the compositions, methods, and kits (e.g., liquid biopsy kits) provided herein may be used to detect and track low-abundance mutations in cancer in order to continuously evaluate MRD, e.g., during treatment. The terms “minimal residual disease” and “MRD,” as may be used interchangeably herein, refer to any remaining cells of a disease or disorder (e.g., cells afflicted with, carrying, spreading, or otherwise compromised by, the disease or disorder (e.g., cancer)) which remain in a subject after the subject is thought to be in remission (e.g., showing no signs or symptoms) of the disease or disorder. Cells associated with MRD may remain in the subject, proliferate, and cause relapse of the disease or disorder in the subject. Since the number of cells associated with MRD is often very low in number and concentration, detection is often difficult, leading to such cells evading detection. Assessing MRD is useful for a variety of reasons, including, for example: determining whether treatment has eradicated the disease or disorder (e.g., cancer); determining whether afflicted, affected, or diseased cells remain; comparing the efficacy of treatments; monitoring remission; assessing or detecting recurrence; choosing treatments; and/or diagnosing disease states. Accordingly, being able to detect and/or quantify MRD is exceptionally clinically relevant. Therefore, effective, and robust methods are needed, which are also cost and time efficient. Shown herein, are methods useful for this application, as well as other applications where detection of rare and/or low concentration nucleic acids (e.g., low-abundance mutations occurring in only a small number of cells contained in a cancer biopsy) are important.

Many approaches have been developed to detect minimal residual disease (MRD). For example, MRD can be assessed using liquid biopsies by tracking tumor mutations in cell-free DNA (cfDNA). Sensitivity can be improved by tracking more mutations per patient. For instance, when tumor fraction is low in the bloodstream, not all mutations will be drawn in a blood tube or it may be the case that a desired cancer-specific mutation is present in such low-abundance, that it evades detection with sequencing. Moreover, MRD typically involves that tracking of numerous individualized mutations. However, tracking large numbers of individualized mutations with sufficient accuracy and efficiency (such that their detection may be relied upon to inform meaningful clinical cancer detection) is challenging due to: 1) the massive excess of normal cfDNA in blood; and 2) the inefficiency of high accuracy sequencing methods.

The inventors contemplated that enriching tumor mutations apart from normal cfDNA could enable high accuracy sequencing of thousands of rare mutations at low cost. Mutation enrichment could also improve MRD detection by enabling more mutations to be tracked and identified in cfDNA.

Searching for thousands of rare mutations in the cfDNA from a blood draw involves scanning millions of DNA bases for potential mutations because a typical blood draw samples a few thousand copies of each gene. While this affords the potential for significant dynamic range in MRD detection, such as detection at 1/1,000,000 tumor fraction, it is intrinsically limited by sequencing errors. For instance, conventional sequencing has an error rate of 1/1000, which means that by using such conventional sequencing, discern true mutations from noise will be difficult when tumor fraction is lower than such threshold.

Higher fidelity sequencing can be achieved by uniquely barcoding each original DNA fragment and sequencing it multiple times to obtain a consensus among reads. For instance, single-strand consensus (SSC) sequencing can achieve 10-fold to 100-fold lower error rates, with greatest improvements realized when combined with noise modeling in many normal samples (Newman et αl.). This works well for sequencing cancer gene panels, but most patients share few mutations in common, and testing of many normal samples is challenging for individualized tests. One way to potentially avoid the need to model noise across normal samples is to require a consensus among SSC reads of the sense strands of each DNA duplex, a technique called duplex sequencing.

Duplex sequencing is one of the most accurate methods for mutation detection (>10-fold more accurate than SSC, Schmitt et αl.) but requires very deep sequencing to recover both strands of each cfDNA duplex. This challenge is magnified for rare mutation detection because not only is deep sequencing required to find the mutation, but also redundant sequencing of each strand is required to suppress errors. For instance, historical review indicates that over 1,000,000x coverage of each mutation site is required to recover most original cfDNA molecules from ~20 nanograms (ng) of cfDNA, and even then, recovery can be incomplete. Techniques have been developed to improve duplex sequencing efficiency, such as by linking sense strands within read pairs (Pel et al.), but still require deep sequencing to find rare mutations.

The inventors contemplated that enriching rare mutations from a duplex sequencing library could improve the efficiency of high accuracy sequencing and that this might be feasible using hybrid capture with short, allele-specific probes. One challenge was that error suppression is different than standard duplex sequencing, given the intrinsic enrichment bias for mutant molecules. However, it was reasoned this might enable a modeling of noise in a more efficient manner, without having to sequence large numbers of normal samples. It was also reasoned that due to how a duplex sequencing library is constructed and amplified, it would be feasible to use just one allele-specific probe per target (e.g., designed to capture either the sense or anti-sense sequence) and still recover library molecules derived from both the sense and anti-sense strands of the original DNA duplex. They also reasoned that it would not be necessary to block wild type sequences to achieve strong enrichment under optimized thermodynamics. Both of these factors would substantially limit the number of probes which would need to be designed.

Accordingly, the disclosure provides a new approach for detecting and/or tracking large numbers of distinct, low-abundance mutations with minimal sequencing required by enriching for low-abundance mutations prior to sequencing, e.g., duplex sequencing. The approach disclosed herein significantly reduces sequencing costs involved in the detection and/or tracking of large numbers of distinct, low-abundance mutations in applications, such as, but not limited to, liquid biopsies for detecting and tracking low-abundance mutations (e.g., using liquid biopsies for monitoring the presence of low-level genetic aberrations or residual genetic information related to a disorder (e.g., cancer), for example, without limitation, minimal residual disease (MRD)). In various aspects, the approach described herein combines hybrid capture using short allele-specific probes with duplex molecular barcoding and noise modeling within each sample to afford high accuracy sequencing of thousands of rare mutations at low cost. The approach described herein demonstrates reliable detection at 1/100,000 tumor fraction using 100-fold less sequencing and the potential to detect 1/1,000,000 by tracking ~ 10,000 individualized mutations.

The disclosure throughout includes common terms used in cell biology, molecular biology, and medicine. Definitions of such terms can be found in can be found in numerous sources, including, but not limited to, “The Merck Manual of Diagnosis and Therapy,” 19th Edition, published by Merck Research Laboratories, 2006 (ISBN 0-911910-19-0); Robert S. Porter et al. (eds.), The Encyclopedia of Molecular Biology, published by Blackwell Science Ltd., 1994 (ISBN 0-632-02182-9). Definitions of common terms in molecular biology can also be found in Benjamin Lewin, Genes X, published by Jones & Bartlett Publishing, 2009 (ISBN-10: 0763766321); Kendrew et al. (eds.), Molecular Biology and

Biotechnology: a Comprehensive Desk Reference, published by VCH Publishers, Inc., 1995 (ISBN 1-56081-569-8) and Current Protocols in Protein Sciences 2009, Wiley Intersciences, Coligan et al., eds. Except where otherwise stated, the present invention was performed using standard procedures, as described, for example in Sambrook et al., Molecular

Cloning: A Laboratory Manual (3 ed.), Cold Spring Harbor Laboratory Press, Cold Spring Harbor, N.Y., USA (2001); and Davis et al., Basic Methods in Molecular Biology, Elsevier Science Publishing, Inc., New York, USA (1995) which are all incorporated by reference herein in their entireties.

The present disclosure also involves next-generation sequencing (NGS) methods (e.g., to conduct duplex sequencing methods described herein) share the common feature of massively parallel, high-throughput strategies. NGS methods can be broadly divided into those that require template amplification and those that do not. Amplification-requiring methods include pyrosequencing commercialized by Roche as the 454 technology platforms (e.g., GS 20 and GS FLX), the Solexa platform commercialized by Illumina, and the Supported Oligonucleotide Ligation and Detection (SOLiD) platform commercialized by Applied Biosystems. Non-amplification approaches, also known as single-molecule sequencing, are exemplified by the HeliScope platform commercialized by Helicos Biosciences, and emerging platforms commercialized by VisiGen, Oxford Nanopore Technologies Ltd., and Pacific Biosciences, respectively. Each of these NGS methods may be employed by and are contemplated to be used in connection with the herein disclosed MAESTRO, which provides a new approach for detecting and/or tracking large numbers of distinct, low-abundance mutations with minimal sequencing required by enriching for low-abundance mutations prior to sequencing, e.g., duplex sequencing.

The present methods, compositions, and kits can be used to detect any mutation, but in particular, may be used to detect low-abundance mutations. The term “low-abundance mutations” may equivalently be referred to as “rare mutations” and/or “low-occurrence mutations” and frequently are associated with somatic mutations arising in cancer in subpopulations of cells. Given such mutations are present in only a subset of cancer cells, their relative abundance in the context of the total amount of isolated nucleic acid from cancer cells is quite low. The term variant allele frequency (VAF) is used to measure the proportion of DNA containing an alteration relative to the total DNA at the same genomic locus. Mutations below 10% VAF, for instance, would generally be regarded as low-abundance, while those below 1% VAF would most certainly be regarded as low-abundance.

Accordingly, in one aspect, the present disclosure provides a method of detecting one or more low-abundance mutations in a sample of DNA duplexes comprising: (a) enriching the sample of DNA duplexes for the one or more low-abundance mutations, wherein the enriching step (a) comprises:
(i) optionally fragmenting the sample of DNA duplexes;
(ii) attaching (e.g., ligating) a unique molecular identifier (UMI) to the top and bottom strands of each of the DNA duplexes to obtain barcoded DNA duplexes;
(iii) amplifying the barcoded DNA duplexes;
(iv) contacting the barcoded DNA duplexes with allele-specific probes specific for one or more low-abundance mutations, thereby enriching the sample of DNA duplexes for the one or more low-abundance mutations, and

(b) sequencing the enriched DNA by duplex sequencing to identify the one or more low-abundance mutations. In a further aspect, the step of duplex sequencing of step (b) results in single-stranded consensus (SSC) sequences of the top or bottom strand sequences and/or double-stranded consensus (DSC) sequences of the top and bottom strand sequences of the barcoded DNA fragments. The one or more low-abundance mutations identified in step (b) can be those mutations that are present on both the top and bottom strands of the double-stranded consensus (DSC) sequences of the barcoded DNA fragments.

In other embodiments, the present disclosure provides a method of detecting one or more low-abundance mutations in a sample of DNA duplexes comprising: (a) enriching the sample of DNA for the one or more low-abundance mutations, wherein the enriching step (a) comprises:
(i) optionally fragmenting the sample of DNA duplexes, if not already fragmented (e.g., such as cfDNA);
(ii) constructing a duplex sequencing library by appending adapters which contain a universal sequence for amplification to obtain barcoded DNA duplexes;
(iii) amplifying the barcoded DNA duplexes;
(iv) contacting the barcoded DNA duplexes with allele-specific probes specific for one or more low-abundance mutations, thereby enriching the sample of DNA for the one or more low-abundance mutations, and

(b) sequencing the enriched DNA by duplex sequencing to identify the one or more low-abundance mutations. In a further aspect, the step of duplex sequencing of step (b) results in single-stranded consensus (SSC) sequences of the top or bottom strand sequences and/or double-stranded consensus (DSC) sequences of the top and bottom strand sequences of the barcoded DNA fragments. The one or more low-abundance mutations identified in step (b) can be those mutations that are present on both the top and bottom strands of the double-stranded consensus (DSC) sequences of the barcoded DNA fragments.

In another aspect, the present disclosure provides a mutation filter designed to protect against the possibility that errors or artifacts (e.g., PCR errors introduced during the amplification step) could arise independently on both top and bottom strands of the barcoded DNA fragments and appear as authentic mutations in the double stranded consensus (DSC) sequences constructed following duplex sequencing of the enriched DNA. Without being bound by theory, the filter works based on the assumptions that (i) errors should be impartial to read family, and (ii) error-prone loci should therefore exhibit a disproportionate number of double-(DSC) to single- (SSC) strand consensus read families bearing mutations. It was found herein that sites with DSC/SSC ratios below 0.15 had poor reproducibility in replicate captures of a non-mutant library (the negative control) (see Examples and FIGS. 13A, 13B). It was also found that the DSC/SSC filter protected against errors introduced by excessive PCR (see Examples and FIGS. 13A, 13B) and further confirmed that MAESTRO probes—which contain the mutant base—do not create false mutant duplexes (see Examples and FIGS. 13A, 13B). Filtering by DSC/SSC ratio was found to be robust to changes in sequencing depth with similar concordance observed at 10% of the original sequencing depth (see Examples and FIGS. 13A, 13B).

Accordingly, the disclosure provides a filter that removes those mutations that are associated with having a disproportionate number of double-stranded consensus (DSC) sequences to single-stranded consensus (SSC) sequences (i.e., a DSC/SSC ratio). In some embodiments, any of the methods of the disclosure further comprise the steps of (1) calculating a double-stranded consensus (DSC) to single-stranded consensus (SSC) ratio (DSC to SSC ratio); (2) and identifying a specific mutation if the DSC to SSC ratio is greater than 0.15. In some embodiments, a DSC to SSC ratio is greater than 0.2. In some embodiments, a DSC to SSC ratio is greater than 0.3.

In another aspect, the disclosure relates to a method of identifying the presence of a specific mutation, comprising: (a) obtaining a pool of DNA duplexes having, suspected of having, or at risk of having the specific mutation in at least one strand, and optionally fragmenting the DNA duplexes; (b) attaching (e.g., ligating) a unique molecular identifier (UMI) to the 5′ and 3′ ends of each strand of the DNA duplexes to produce tagged duplexes, wherein the UMIs are unique to each tagged duplex; (c) amplifying the tagged duplexes by polymerase chain reactions (PCR) to produce amplified duplexes; (d) denaturing the amplified duplexes to produce single-stranded amplified DNA; (e) capturing single-stranded amplified DNA having the specific mutation using an allele-specific probe that anneals to the specific mutation to produce an enriched sample; (f) sequencing the enriched sample; and (g) confirming the presence of the specific mutation if the specific mutation is observed in both strands of the tagged duplex as identified by the UMIs.

In some aspects, the disclosure relates to a method comprising: (a) obtaining a pool of DNA duplexes comprising a specific mutation in at least one strand and attaching (e.g., ligating) a unique molecular identifier (UMI) to the 5′ and 3′ ends of each strand of the DNA duplexes to produce tagged duplexes, wherein the UMIs are specific to each tagged duplex; (b) amplifying the tagged duplexes by polymerase chain reactions (PCR) to produce amplified duplexes and subsequently denaturing the amplified duplexes to produce single-stranded amplified DNA; (c) capturing single-stranded amplified DNA having the specific mutation using an allele-specific probe that anneals to the specific mutation to produce an enriched sample, and sequencing the enriched sample; and (d) calculating a double-stranded consensus (DSC) to single-stranded consensus (SSC) ratio (DSC to SSC ratio) using the UMIs, and identifying the specific mutation if the DSC to SSC ratio is greater than 0.15.

In some embodiments, an allele-specific probe of any of the methods of the disclosure anneals to the specific mutation at between 48° C. and 52° C. and the probe is recovered, to produce a sample that is enriched for single-stranded amplified DNA having the specific mutation.

In some embodiments, any of the methods of the disclosure further comprise the steps of (1) calculating a double-stranded consensus (DSC) to single-stranded consensus (SSC) ratio (DSC to SSC ratio); (2) and identifying a specific mutation if the DSC to SSC ratio is greater than 0.15. In some embodiments, a DSC to SSC ratio is greater than 0.2. In some embodiments, a DSC to SSC ratio is greater than 0.3.

In some embodiments, an allele-specific probe of any of the methods of the disclosure is about 10 to about 60 nucleotides long. In some embodiments, an allele-specific probe of any of the methods of the disclosure is about 15 to about 50 nucleotides long. In some embodiments, an allele-specific probe of any of the methods of the disclosure is about 20 to about 40 nucleotides long. In some embodiments, an allele-specific probe of any of the methods of the disclosure is about 28 to about 32 nucleotides long. In some embodiments, an allele-specific probe of any of the methods of the disclosure is 30 nucleotides long.

In some embodiments, a specific mutation of any of the methods of the disclosure can be identified with at least 10 times fewer sequencing reads as compared with conventional duplex sequencing methods. In some embodiments, a specific mutation of any of the methods of the disclosure can be identified with at least 100 times fewer sequencing reads as compared with conventional duplex sequencing methods.

In some embodiments, in any of the methods of the disclosure, capturing of the single-stranded amplified DNA having the specific mutation using an allele-specific probe that anneals to the specific mutation is repeated on the enriched sample at least 10 times relative to a control. In some embodiments, in any of the methods of the disclosure, capturing of the single-stranded amplified DNA having the specific mutation using an allele-specific probe that anneals to the specific mutation is repeated on the enriched sample at least 100 times relative to a control. In some embodiments, in any of the methods of the disclosure, capturing of the single-stranded amplified DNA having the specific mutation using an allele-specific probe that anneals to the specific mutation is repeated on the enriched sample at least 1,000 times relative to a control.

In some embodiments, a pool of any of the methods of the disclosure is generated from a liquid biopsy. In some embodiments, a liquid biopsy is conducted on a subject or on a sample from a subject.

In some embodiments, a subject of any of the methods of the disclosure has a tumor, had a tumor in the past, or is suspected of having a tumor. In some embodiments, a subject of any of the methods of the disclosure has breast cancer, had breast cancer in the past, or is suspected of having breast cancer. In some embodiments, a subject of any of the methods of the disclosure is undergoing, has undergone, or will undergo, neoadjuvant therapy for early-stage breast cancer. In some embodiments, a subject of any of the methods of the disclosure is postoperative.

In some embodiments, a liquid biopsy of any of the methods of the disclosure contains cell-free DNA (cfDNA). In some embodiments, a liquid of any of the methods of the disclosure biopsy is genome-wide.

In some embodiments, a method of the disclosure is a method for detecting minimal residual disease (MRD). In some embodiments, a method of the disclosure is a method for detecting a single nucleotide polymorphism (SNP). In some embodiments, a SNP is in the germ line. In some embodiments, a method of the disclosure is a method for detecting at least one insertion or deletion. In some embodiments, a method of the disclosure is a method for detecting at least one structural variant.

In some embodiments, a pool of the disclosure is enriched for more than one specific mutation. In some embodiments, a pool of the disclosure is enriched for at least 25 specific mutations. In some embodiments, a pool of the disclosure is enriched for at least 50 specific mutations. In some embodiments, a pool of the disclosure is enriched for at least 100 specific mutations. In some embodiments, a pool of the disclosure is enriched for at least 500 specific mutations. In some embodiments, a pool of the disclosure is enriched for at least 1,000 specific mutations.

In some embodiments, a method of the disclosure is capable of tracking up to 10,000 distinct, low-abundance specific mutations throughout the genome.

In some embodiments, mutations of the disclosure are in non-overlapping regions of the genome.

In some embodiments, an allele-specific probe of the disclosure is biotinylated.

In some embodiments, a method of the disclosure, further comprises selecting low-noise mutations. In some embodiments, low-noise mutations comprise mutations at sites in a reference sequence comprising an adenine (A) and thymine (T) base pairing.

In some embodiments, a pool of the disclosure includes internal controls. In some embodiments, internal controls of the disclosure comprise synthetic mutants that the allele-specific probes are capable of binding.

In some embodiments, performance of an allele-specific probe of the disclosure can be assessed based on its ability to detect synthetic mutants.

In some embodiments, an internal control of the disclosure is included for each specific mutation or duplex in the pool.

In some embodiments, an allele-specific probe of the disclosure comprises a modification. In some embodiments, a modification improves structural stability of the probe. In some embodiments, a modification improves binding affinity.

In some embodiments, an allele-specific probe of the disclosure comprises a minor groove binder (MGB). In some embodiments, an MGB is attached to the 3′ end of the allele-specific probe.

In some embodiments, a recovery moiety is attached to the 5′ end of an allele-specific probe of the disclosure.

In some embodiments, a recovery moiety is biotin.

In some aspects, the disclosure relates to a method of detecting minimal residual disease, comprising: (a) performing a liquid biopsy on a subject having, suspected of having, at risk of having, or who has previously had cancer; and (b) performing any of the method of the disclosure for detecting or identifying a specific mutation; wherein identification of mutations associated with tumors indicates minimal residual disease.

In some embodiments, an allele-specific probe of a method of the disclosure, comprises a nucleotide complementary to a specific mutation, wherein the nucleotide complementary to a specific mutation is in the middle 50% of nucleotides of the allele-specific probe. In some embodiments, an allele-specific probe of a method of the disclosure, comprises a nucleotide complementary to a specific mutation, wherein the nucleotide complementary to a specific mutation is in the middle 34% of nucleotides of the allele-specific probe. In some embodiments, an allele-specific probe of a method of the disclosure, comprises a nucleotide complementary to a specific mutation, wherein the nucleotide complementary to a specific mutation is in the middle 5% of nucleotides of the allele-specific probe.

In some embodiments, the Gibbs free energy (ΔG) of an allele-specific probe of a method of the disclosure annealing to its complementary sequence is at least -20 kcal/mol at Temp =50° C., but no more than -12 kcal/mol at Temp =50° C. In some embodiments, the Gibbs free energy (ΔG) of an allele-specific probe of a method of the disclosure annealing to its complementary sequence is at least -18 kcal/mol at Temp =50° C., but no more than -14 kcal/mol at Temp =50° C.

In some embodiments, the sequence of an allele-specific probe is 100% homologous with less than 10 sequences of a reference genome of the subject. In some embodiments, the sequence of an allele-specific probe is 100% homologous with less than 5 sequences of a reference genome of the subject.

In some aspects, the disclosure relates to a method of making an allele-specific probe, the method comprising: (a) identifying a specific mutation in a nucleic acid sequence of a genome; (b) generating a complementary nucleic acid (CNA) including a complementary base to the specific mutation; and (c) attaching a recovery moiety to the 5′ nucleotide of the allele-specific probe; wherein the complementary base is in the middle 50% of nucleotides of the CNA; wherein, the CNA comprises at least 12, but no more than 60 nucleotides; wherein the Gibbs free energy of the CNA and the nucleic acid comprising the specific mutation is at least -20, but no more than -12; wherein the annealing temperature of the allele-specific probe is at least 48° C. (°C), but no more than 52° C.; and wherein the CNA is 100% homologous with less than 10 sequences within the genome. In some embodiments, the disclosure relates to an allele-specific probe produced by the method of making an allele-specific probe. In some embodiments, any of the methods of the disclosure may use the allele-specific probe, made by the method of making an allele-specific probe.

These and other aspects and embodiments will be described in greater detail herein. The description of some exemplary embodiments of the disclosure are provided for illustration purposes only and not meant to be limiting. Additional compositions and methods are also embraced by this disclosure.

The summary above is meant to illustrate, in a non-limiting manner, some of the embodiments, advantages, features, and uses of the technology disclosed herein. Other embodiments, advantages, features, and uses of the technology disclosed herein will be apparent from the Detailed Description, Drawings, Examples, and Claims.

### BRIEF DESCRIPTION OF THE DRAWINGS

FIGS. 1A-1D show an overview and results of the MAESTRO workflow technique. FIG. 1A shows the MAESTRO workflow of identifying somatic SNVs, designing for strong candidates, enriching the mutant duplex nucleic acids, and duplex sequencing with error suppression. FIG. 1B shows a comparison of allele fractions using mutation enrichment with MAESTRO against conventional hybrid capture. The same tumor benchmarking sample (0.1% tumor/normal) was used in both cases and in subsequent figures. FIG. 1C shows mutant molecule concordance between MAESTRO and conventional hybrid capture. FIG. 1D shows the sequencing requirement to saturate mutant molecule recovery using MAESTRO against conventional hybrid capture.

FIGS. 2A-2B show dilution benchmarking. FIG. 2A shows a comparison of the signal (i.e., number of mutations) seen in multiple replicates of 2 tumor dilutions (e.g., 1:100,000 and 1:1,000,000) to the signal seen in multiple replicates of a negative control. FIG. 2B shows the quantification of the mutation abundance across multiple inputs and varying tumor dilutions from 1:10 down to 1:10,000,000. For the 1:1,000 dilution, conventional hybrid capture was also applied to inputs from 5 nanogram (ng) to 250 ng and results are annotated as stars.

FIG. 3 shows an application of MAESTRO to patients treated for breast cancer.

FIGS. 4A-4E show an outline and overview of the workflow and experimental evaluation of MAESTRO. FIGS. 4A and 4B provide a background and description of the technological challenges and need for increased sensitivity as described herein. FIG. 4C provides an overview of tracking low-noise mutations in MAESTRO to increase sensitivity. FIG. 4D provides a conclusion summary of non-limiting examples of the aspects of MAESTRO. FIG. 4E shows data relating to the number of cancer cells over time with relative detection levels of non-limiting examples of method of detection.

FIG. 5 shows that MAESTRO enables accurate, low-cost mutation tracking in clinical specimens. The top panel shows that up to 10,000 MAESTRO probes are designed with stringent length and ΔG for single-nucleotide discrimination of predefined mutations (FIG. 10). DNA libraries containing uniquely barcoded top and bottom strands are subject to hybrid capture using allele-specific MAESTRO probes. Only molecules containing tracked mutations are captured and sequenced with duplex consensus for error suppression. The bottom panel shows that while using MAESTRO the same mutations are discovered using up to 100 x less sequencing because uninformative regions are depleted.

FIGS. 6A-6B show that MAESTRO uncovers most mutant duplexes using significantly fewer reads. FIG. 6A shows a comparison of variant allele frequency with conventional duplex sequencing to MAESTRO with 438 probe panel at 1/1k tumor fraction. FIG. 6B shows a downsampling of conventional duplex sequencing and MAESTRO. As an inset, mutant duplex overlap is shown; of the 57 mutant duplexes exclusive to Conventional, 42 were detected by MAESTRO but excluded by the noise filter. The initial sample was barcoded with UMIs (unique molecular indices) which allowed for tracking individual duplex molecules through different experimental conditions.

FIGS. 7A-7B show the MAESTRO fingerprint validation of whole exome tumor samples. FIG. 7A shows the performance of 16 x tumor fingerprints using both Conventional and MAESTRO. Mutations were called from the 16 x tumor biopsies and both Conventional and MAESTRO fingerprints were created for all possible mutations from each tumor. The tumor biopsy libraries were captured with the Conventional and MAESTRO fingerprints and duplexes were sequenced. Fingerprints were split into two groups based on whether or not their original tumor VAF was < 10%. A mutation was considered validated if it was observed in the sequenced duplexes of the Conventional or MAESTRO sample. FIG. 7B is a graph comparing variant allele fraction across all mutations from all Conventional and MAESTRO panels.

FIGS. 8A-8B show that MAESTRO can detect signal above noise at 1/100k tumor fraction. FIG. 8A shows mutations detected in MAESTRO using a 438 probe panel across 18 x biological replicates of a 1/100k dilution and 17 x biological replicates of a negative control. FIG. 8B shows mutations detected in MAESTRO using a 10,000 probe panel across 16 x biological replicates of a 1/100k dilution, 17 x biological replicates of 1/1M, and 12 x negative controls. The Welch’s t-test was used to determine whether significantly more mutations were uncovered in each tumor dilution compared to the negative controls.

FIG. 9 shows MAESTRO improves detection of MRD in pre-operative setting. The patient graphs show genome-wide tumor mutations detected with MAESTRO compared to exome-wide tumor mutations detected with a personalized MRD test built on conventional duplex sequencing. Fingerprint sizes for the two conditions are shown with triangles. Mutations from all patients were combined into a single panel for MAESTRO and the same panel was applied to all samples. The heatmap shows mutation counts detected using MAESTRO with patient-specific mutations on the diagonal and highlights MAESTRO’s specificity.

FIG. 10 provides a probe design overview.

FIG. 11 shows probe characteristics effect on enrichment. Showing results from the 1/1k dilution samples where each data point is a probe within the capture panel. Enriched VAF is plotted as a function of different probe sequence characteristics.

FIGS. 12A-12C show probe and hybridization optimization. FIG. 12A shows the effect of varying probe length and hybridization temperature on enrichment performance measured using variant allele fraction (VAF), on target fraction, and recall. All temperatures were tested for each probe length, but only the best performing temperature is shown. Data points for VAF and recall show mean across 20 sites whereas on target is calculated once per sample (total bases on target / total bases sequenced). FIG. 12B provides an IGV screenshot showing an example of recall. Here, the same sample was captured using conventional and MAESTRO and identical source duplexes are shown. Recall in this example is ⅚ as 5 of the conventional duplexes were seen in the MAESTRO condition. FIG. 12C shows that when designing probes, either the top or the bottom strand can be used. There will be different mismatches between the probe and wildtype base depending on which strand is chosen. Here, for each reference base across 144 sites, a MAESTRO probe was designed for either the top or the bottom strand and VRF performance is shown. When the reference base is a “C” it is beneficial to design for the negative strand. In all other cases, the positive strand is optimal. Showing mean with error bars representing 95% confidence interval.

FIGS. 13A-13C show a tunable MAESTRO filter to correct for PCR errors. FIG. 13A shows that library molecules accumulate polymerase errors during PCR. In conventional capture, PCR errors are suppressed by sequencing through all molecules at a given site, mutated or not. Errors can be corrected because they are seen spuriously and do not pass single strand consensus (SSC). With MAESTRO probes, PCR errors at the target base are also captured and sequenced. If an unmutated library molecule acquires the same PCR error on fragments derived from both the top and bottom strand of the same starting molecule, a false mutation is called even after double strand consensus (DSC). Additionally, FIG. 13A also provides that in order to filter rare PCR errors that make it through duplex consensus, a DSC/SSC filter can be applied. To verify a mutation is real, most SSCs at the mutant site must be involved in forming a DSC (ideal DSC/SSC ratio of 0.5). Because PCR errors are impartial to read family, an accumulation of unpaired SSCs without accompanying DSC support signals a false mutation. FIG. 13B shows a MAESTRO locus specific noise filter applied to four replicate negative controls. Molecules shared in at least two replicates are shown as well as molecules exclusive to one replicate. After applying the noise filter the majority of exclusive molecules are removed and shared molecules are retained. FIG. 13C shows a comparison of a sample with no added cycles of PCR to the same sample but with 40 added cycles before and after incorporating the DSC/SSC noise filter. Samples in both C and D used the 10,000 SNV panel.

FIGS. 14A-14B show a probe spike-in experiment. FIG. 14A is a schematic showing how probes contain mutation of interest and may have the ability to create mutant duplexes. In order for a mutation to be called after duplex consensus, evidence must be seen in molecules derived from both the original top and bottom strand. During the 16 cycles of PCR performed after capture, a MAESTRO probe could bind to a non-mutant fragment and extend (1). This extended probe could be amplified in the next few rounds of PCR using the Illumina primers present in post-capture PCR (2). The copied products contain the mutation but are not able to be sequenced (3). These products can then bind to another unmutated fragment and extend (4). This creates a mutant molecule with both adapters intact that can be sequenced (5). This can result in a false-positive during duplex consensus if the same events happen on the other strand (6). FIG. 14B shows Capture was performed using the 10,000 SNV MAESTRO panel on two replicate negative control samples (no spike-in) and compared to the same negative controls with 1,000 X the standard concentration of ten MAESTRO probes added prior to both post-capture PCRs (1,000 X spike-in).

FIGS. 15A-15B show the downsampling DSC/SSC ratio. FIG. 15A shows a MAESTRO locus specific noise filter applied to four replicate negative controls with downsampling ranging from 1.0 (full sequencing depth used) down to 0.05 of the original depth. The samples and definitions are as described in FIG. 11. FIG. 15B provides a direct comparison of the fraction of duplexes passing DSC/SSC ratio filter at 1.0 (full sequencing depth) compared to 0.05 of the original depth.

FIGS. 16A-16D show benchmarking 1/100k dilutions, and all use 18 x replicates of a 1/100k dilution and 17 x replicates of a negative control with a 438 SNV panel. FIG. 16A shows a comparison of downsampling curves resulting from applying conventional duplex sequencing and MAESTRO to the same replicate samples. FIG. 16B shows the distance from mutation site to fragment end (using the end closest to the mutation) shown for all mutant molecules uncovered with conventional and MAESTRO. Molecules with mutation near fragment ends were efficiently captured with MAESTRO probes but were not captured with conventional probes. FIG. 16C shows how removing molecules near fragment ends compensates for the different capture efficiencies of conventional and MAESTRO probes and results in high concordance between the two methods. Each axis contains the mutation counts seen across replicates. Points are shaded based on the number of replicates that overlap and any data point with more than one replicate is annotated with a number. FIG. 16D shows how with single strand consensus sequencing, many additional mutations are uncovered in the negative control making it difficult to distinguish signal from noise.

FIGS. 17A-17B show a validation of false positives in negative controls. FIG. 17A shows a validation experiment design. FIG. 17B shows a duplex molecular concordance of false positives seen across 12 negative controls with conventional duplex sequencing and MAESTRO.

FIGS. 18A-18C show MRD testing in a Phase II study of preoperative doxorubicin and cyclophosphamide followed by paclitaxel with avastin in triple-negative breast cancer. FIG. 18A shows a treatment course for patients from diagnosis to surgery with time of blood draw annotated. FIG. 18B shows a whole-exome sequencing of patients’ tumor biopsies was performed, and individualized MRD tests were applied using conventional duplex sequencing to serial cfDNA time points as previously described. MRD status (>=2 mutations detected) is indicated. Stars denote the four patients selected for more extensive testing with MAESTRO, results of which are shown in FIGS. 8A-8B. FIG. 18 provides a comparison of tumor fractions from T1 and T2 blood draws. Data points are shown by pathological complete response or patients having residual cancer burden. Circles indicate patients that experienced recurrence. Error bars indicate 95% confidence intervals.

FIG. 19 shows probe design success rates. Probe design success rate for the 4 patient-specific fingerprints analyzed in FIG. 9. Here, “Exonic” mutations were derived from whole exome sequencing of the tumor whereas “Exonic + Intronic” were from the combined output of whole exome and whole genome sequencing of the patient’s tumor.

FIG. 20 shows somatic SNV counts and validation using patient’s tumor DNA. The total SNV counts from WGS is shown for each patient along with the total number of SNVs that pass our specificity filter that ensures good mappability. Next is the total number of SNVs that pass MAESTRO probe design and lastly are the total counts of mutations that were validated in each patient’s tumor DNA.

FIG. 21 shows MAESTRO tumor fraction estimation. The estimated tumor fraction was compared to the actual tumor fraction for a spike-in tumor dilution series, and the estimated tumor fractions were calculated.

FIG. 22A shows a coiling indouble helix or duplex of DNA. FIG. 22B shows an x-ray crystal structure of a 1:1 complex of netropsin:DNA (PDB 121D on the top, and an x-ray crystal structure of a 2:1 complex of distamycin:DNA (PDB 378D) on the bottom. FIG. 22C shows structures of commonly studied minor groove binders, including natural and synthetic molecules with diverse structures.

FIG. 23A shows a larger ΔΔG (greater discrimination) at MGB binding site. Mismatch discrimination with ODN1 (±MGB). UV melting curves from the DNA duplexes were used to calculate a free energy difference (ΔΔ°so) for each mismatch type and location. Mismatch discrimination for each duplex is shown graphically in relation to the MGB region. FIG. 23B shows that MGB probes show specificity at limiting dilutions. Titration of PCR template with genomic DNA background. 100 000 to 1 copies of the match plasmid per PCR reaction were detected using the MGB 15mer probe. 200 ng of herring sperm genomic DNA was added to each reaction. Flourescence at cycle 1 was subtracted from each curve using the manufacturer’s software. 200 ng = 40,000 copies. FIG. 23C shows MGB’s level Tm of probes across GC content. Tₘ comparison of fluorogenic MGB probes and no-MGB ODNs. Tₘ of match and mismatch complements for sequences with representative G+C content are plotted. FIG. 23D shows a chemical structure where DPI3 = dihydrocyclopyrroloindole tripeptide. The linker region may also affect how the MGB performs (on either N or C terminus).

FIG. 24 shows the SNP site in an MGB probe.

FIG. 25 shows MAESTRO vs. MGB probes.

FIG. 26 shows the capture plan. Per locus, 4 probes x 8 temperatures = 32 hyb conditions, with the hybridization temperature ranging from 60° C. to 75° C. Both loci were captured in each well, and a sampling of single and double capture for ddPCR was performed.

FIGS. 27A-27C show the creation of M AESTRO panels. MGB can only be added to 3′ end, and the Thermo Fisher requirements are 3′ MGB, 5′ biotin, and 13-30 nucleotides.

FIG. 28 shows an approach to create MAESTRO probes and internal controls simultaneously from one pool of synthetic oligos.

FIG. 29 provides a detailed schematic of how internal controls would be created to spike into samples to be tested with MAESTRO.

FIG. 30 shows that each collection of internal controls for a single mutation comprises a diversity of molecules with different indices. The number of indices observed per locus after sequencing is used to estimate the capture efficiency of each probe. This, in turn, may be used to ‘validate’ the performance of each MAESTRO probe.

### DETAILED DESCRIPTION

The disclosure provides new methods, compositions, and kits for detecting and/or tracking large numbers of distinct, low-abundance mutations with minimal sequencing required by enriching for low-abundance mutations prior to sequencing, e.g., duplex sequencing. Aspects of the disclosure relate to a novel method referred to as: minor allele enrichment sequencing targeting rare occurrences (MAESTRO). This method combines hybrid capture using short allele-specific probes with duplex molecular barcoding and noise modeling within each sample to afford high accuracy sequencing of thousands of rare mutations at low cost. Such methods may be useful for a variety of applications, including monitoring the presence of low-level genetic aberrations or residual genetic information related to a disorder (e.g., cancer), for example, without limitation, minimal residual disease (MRD). The terms “minimal residual disease” and “MRD,” as may be used interchangeably herein, refer to any remaining cells of a disease or disorder (e.g., cells afflicted with, carrying, spreading, or otherwise compromised by, the disease or disorder (e.g., cancer)) which remain in a subject after the subject is thought to be in remission (e.g., showing no signs or symptoms) of the disease or disorder. Cells associated with MRD may remain in the subject, proliferate, and cause relapse of the disease or disorder in the subject. Since the number of cells associated with MRD is often very low in number and concentration, detection is often difficult, leading to such cells evading detection. Assessing MRD is useful for a variety of reasons, including, for example: determining whether treatment has eradicated the disease or disorder (e.g., cancer); determining whether afflicted, affected, or diseased cells remain; comparing the efficacy of treatments; monitoring remission; assessing or detecting recurrence; choosing treatments; and/or diagnosing disease states. Accordingly, being able to detect and/or quantify MRD is exceptionally clinically relevant. Therefore, effective, and robust methods are needed, which are also cost and time efficient. Shown herein, are methods useful for this application, as well as other applications where detection of rare and/or low concentration nucleic acids are important.

### Methods

Accordingly, in some aspects, the disclosure relates to a method of identifying the presence of a specific mutation, comprising: (a) obtaining a pool of DNA duplexes having, suspected of having, or at risk of having the specific mutation in at least one strand, and optionally fragmenting the DNA duplexes; (b) attaching (e.g., ligating) a unique molecular identifier (UMI) (e.g., as part of an adapter molecule) to the 5′ and 3′ ends of each strand of the DNA duplexes to produce tagged duplexes, wherein the UMIs are unique to each tagged duplex; (c) amplifying the tagged duplexes by polymerase chain reactions (PCR) to produce amplified duplexes; (d) denaturing the amplified duplexes to produce single-stranded amplified DNA; (e) capturing single-stranded amplified DNA having the specific mutation using an allele-specific probe that anneals to the specific mutation to produce an enriched sample; (f) sequencing the enriched sample; and (g) confirming the presence of the specific mutation if the specific mutation is observed in both strands of the tagged duplex as identified by the UMIs.

In some aspects, the disclosure relates to a method comprising: (a) obtaining a pool of DNA duplexes comprising a specific mutation in at least one strand and attaching (e.g., ligating) a unique molecular identifier (UMI) to the 5′ and 3′ ends of each strand of the DNA duplexes to produce tagged duplexes, wherein the UMIs are specific to each tagged duplex; (b) amplifying the tagged duplexes by polymerase chain reactions (PCR) to produce amplified duplexes and subsequently denaturing the amplified duplexes to produce single-stranded amplified DNA; (c) capturing single-stranded amplified DNA having the specific mutation using an allele-specific probe that anneals to the specific mutation to produce an enriched sample, and sequencing the enriched sample; and (d) calculating a double-stranded consensus (DSC) to single-stranded consensus (SSC) ratio (DSC to SSC ratio) using the UMIs, and identifying the specific mutation if the DSC to SSC ratio is greater than 0.15.

The term “specific mutation,” as may be used herein, refers to a change, alteration, or modification to a nucleotide in a nucleic acid as compared to its wild-type sequence (e.g., unmutated, reference sequence), which is targeted by a probe of the disclosure and is of interest. For example, a specific mutation may be known to be associated with a disorder (e.g., disease or condition). As such, evaluating a subject, or sample from a subject (e.g., pool of DNA duplexes) for the presence of a specific mutation, or evaluating the same for identification of any of such specific mutations, may be useful in, without limitation, the diagnosis, treatment, and/or evaluation of a subject. In some embodiments, of the disclosure, the identification and or presence of a specific mutation is used to indicate the presence of nucleic acids (e.g., DNA, cfDNA) related to a disorder. In some embodiments, the method of the disclosure use this determination to indicate and/or evaluate a subject for minimal residual disease (MRD).

Without limitation, mutations may include substitutions, insertions, deletions, or any combination of the same. In some embodiments, there at least one mutation. In some embodiments, there are more than one mutation. In some embodiments, where there is more than one mutation, the mutations are distinct (e.g., not of the same type (e.g., substitutions, insertions, deletions)). In some embodiments, where there is more than one mutation, the mutations are the same (e.g., not of the same type (e.g., substitutions, insertions, deletions)). Additionally, in some embodiments, mutations result in a frameshift. In some embodiments, a mutation comprises a single nucleotide polymorphism (SNP). In some embodiment a mutation is a structural variant. As used herein, a structural variant shall refer to a variation in structure of a chromosome of a subject, such variation can comprise many kinds of variation in the genome of a subject. For example, without limitation, structural variations can includes microscopic and submicroscopic alterations, such as deletions, duplications, copy-number variants, insertions, inversions and translocations. In some embodiments, a mutation occurs in one strand of a nucleic acid duplex. In some embodiments, the strand is the plus strand (e.g., ‘+’, sense strand). In some embodiments, the strand is the negative strand (e.g., ‘-’, antisense strand). In some embodiments, a mutation occurs in both strands of a nucleic acid duplex (e.g., ‘+’ and ‘-’ strands). In some embodiments, a mutation is a mutation known to be associated with a cancer. In some embodiments, a cancer is leukemia. In some embodiments, a mutation is known to be related, or originated in, tumor tissue.

In some embodiments, specific mutations are chosen (e.g., established as targets) based on existing information such as literature presenting lists of known mutations, databases of known mutations, and/or any other sources of known mutations. In some embodiments, specific mutations are chosen from existing information about a subject (e.g., the subject from which the pool of DNA duplexes and/or enriched sample will be obtained). For example, the existing information may be subject history of disease or disorder, or subject history of a specific mutation. In some embodiments, a specific mutation is chosen based on known association with a disease or disorder. In some embodiments, a specific mutation is chosen based on the fact that a subject has, is suspected of having, or has had a disease of which the specific mutation is associated or related. In some embodiments, a specific mutation is chosen based on existing information or sequencing data from a tissue sample of a subject (either presently obtained or obtained in the past). In some embodiments, the tissue sample is tumor tissue.

In some embodiments, a pool of DNA duplexes (“a pool”) is obtained from a sample. As used in the methods herein, a sample may be any sample from a subject. For example, without limitation, blood, skin, tissue, hair, saliva, bodily fluid, cells, or any other biological component from which the skilled artisan may ascertain, using techniques known and readily available in the art, the parameter being evaluated (e.g., presence or absence of nucleic acids containing a specific mutations or duplexes containing the same). In some embodiments, a sample is a blood sample. In some embodiments, a blood sample contains cell-free DNA (“cfDNA”).

The term “subject,” as used herein, refers to any organism in need of treatment or diagnosis using the subject matter herein. For example, without limitation, subjects may include mammals and non-mammals. In some embodiments, a subject is mammalian. In some embodiments, a subject is non-mammalian. As used herein, a “mammal,” refers to any animal constituting the class Mammalia (e.g., a human, mouse, rat, cat, dog, sheep, rabbit, horse, cow, goat, pig, guinea pig, hamster, chicken, turkey, or a non-human primate (e.g., Marmoset, Macaque)). In some embodiments, a mammal is a human. In some embodiments, a subject is under the care and/or direction of a medical professional (e.g., a patient). In some embodiments, a subject is a patient. In some embodiments, a subject has, is at risk of having, has had previously, or is suspected of having a disorder (e.g., disease). In some embodiments, a subject is a subject that has a tumor, a subject that had a tumor in the past, a subject at risk of having a tumor, or a subject that is suspected of having a tumor. In some embodiments, a tumor is cancerous. In some embodiments, a disorder is associated or related to mutations in nucleic acids. In some embodiments, a disorder is a cancer. In some embodiments, a cancer is leukemia. In some embodiments, a cancer is breast cancer.

In some embodiments, a sample is acquired by biopsy. In some embodiments, a biopsy is a liquid biopsy. Liquid biopsies are well-known in the field to the skilled artisan. They are generally known to be liquid or fluid phase biopsies where the sampling and analysis is that of non-solid biological matter from a subject (e.g., bodily fluid, blood, saliva, etc.). A sample from the liquid biopsy is then analyzed for the presence of markers (e.g., specific mutations or nucleic acids and/or duplexes bearing specific mutations or sequences). The component of the fluid may vary depending on the target to be analyzed, for example, circulating tumor cells and/or circulating tumor DNA (ctDNA), circulating endothelial cells, cell-free DNA (cfDNA), and/or cell-free fetal DNA (cffDNA). In some embodiments, a liquid biopsy sample is a blood sample. In some embodiments, a liquid biopsy is of the reproductive cells of a subject (e.g., from eggs or spermatozoa). In some embodiments, cfDNA is targeted by the methods of the disclosure. However, any suitable liquid biopsy may be used with the methods herein as can be determined by the skilled artisan without undue experimentation.

Once the sample is obtained (e.g., acquired), a pool of DNA duplexes is established using the sample. A “pool of DNA duplexes,” as may be used herein, refers to a plurality of DNA duplexes (e.g., double-stranded nucleic acids) in the sample. The term “DNA duplex,” as may be used herein, refers to an individual double-stranded nucleic acid molecule. As such, the term shall be understood to include genomic DNA (gDNA), germline DNA, cell-free DNA, and other forms of DNA provided the molecule comprise two annealed strands for at least a portion of the nucleic acid molecule. Accordingly, a DNA duplex may refer to an intact DNA molecule comprising an entire genome, portion thereof, or fragments thereof (e.g., after fragmenting, shearing), provided the molecule remains double-stranded for at least a portion of the nucleic acid molecule.

In some embodiments, DNA duplexes of a pool are fragmented. This fragmentation breaks apart a nucleic acid into small fragments. In some embodiments, a DNA duplex is fragmented to reduce its size. In some embodiments, a DNA duplex is fragmented to make a pool of DNA duplexes more homogenous with respect to the size of DNA duplexes therein. In some embodiments, a DNA duplex is fragmented to produce fragments of about 50 to about 250 bases pairs in length (e.g., about 50 to about, 51, 52, 53, 54, 55, 56, 57, 58, 59, 60, 61, 62, 63, 64, 65, 66, 67, 68, 69 ,70, 71, 72, 73, 74, 75, 76, 77, 78, 79, 80, 81, 82, 83, 84, 85, 86, 87, 88, 89,90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250 base pairs in length). In some embodiments, a DNA duplex is fragmented to produce fragments of about 100 to about 200 bases pairs in length. In some embodiments, a DNA duplex is fragmented to produce fragments of about 120 to about 180 bases pairs in length. In some embodiments, a DNA duplex is fragmented to produce fragments of about 130 to about 170 bases pairs in length. In some embodiments, a DNA duplex is fragmented to produce fragments of about 140 to about 160 bases pairs in length. In some embodiments, a DNA duplex is fragmented to produce fragments of about 150 base pairs in length. In some embodiments, a DNA duplex is already fragmented, e.g. cell-free DNA from blood plasma.

Fragmentation may be accomplished, physically (e.g., by sonication or physical force), enzymatically, or chemically. However, all forms of fragmentation inherently damage the strands to break them into smaller portions. Methods of fragmentation are well-known in the art and will be readily appreciated and selected by the skilled artisan. In some embodiments, prior to step (a) a sample has been: (i) fragmented; or (ii) cleaved and tagged (tagmented). In some embodiments, fragmentation is by: (a) physical fragmentation; (b) enzymatic fragmentation; and/or (c) chemical fragmentation. In some embodiments, fragmentation is by physical fragmentation. In some embodiment, physical fragmentation is by nebulization. In some embodiments, physical fragmentation is by acoustic shearing. In some embodiments, physical fragmentation is by needle shearing. In some embodiments, physical fragmentation is by French pressure cell. In some embodiments, physical fragmentation is by sonication. In some embodiments, physical fragmentation is by hydrodynamic shearing. In some embodiments, fragmentation is by enzymatic fragmentation. In some embodiments, enzymatic fragmentation is by nuclease or endonuclease. In some embodiments, enzymatic fragmentation is by DNase I. In some embodiments, enzymatic fragmentation is by restriction endonuclease. In some embodiments, enzymatic fragmentation is by transposase. In some embodiments, is by chemical fragmentation. In some embodiments, chemical fragmentation is by heat and divalent metal cation fragmentation.

Once a DNA duplexes is fragmented, unique molecular identifiers (UMls) may be ligated to one or both ends of the DNA duplex as part of a sequencing adapter which contains sequences to facilitate primer binding and amplification. This process of sequencing preparation is well established in the art, while there are also other ways to append sequencing adapters comprising UMIs. UMIs are tags (e.g., specific sequences) which may be useful in identifying a strand and/or its duplex counterpart (e.g., complementary strand) throughout the remainder of the method and during any post sequencing processing and/or evaluation (e.g., analysis). In some embodiments, UMIs are contained within a sequencing adapter. Use of UMIs is well-known throughout the field. In some embodiments, a UMI is attached to at least a 5′ end of at least one strand of a DNA duplex. In some embodiments, a UMI is attached both 5′ ends of a DNA duplex. In some embodiments, a UMI is attached to at least a 3′ end of at least one strand of a DNA duplex. In some embodiments, a UMI is attached both 3′ ends of a DNA duplex. In some embodiments, a UMI is attached to at least each of, a 5′ end of at least one strand of a DNA duplex, and a 3′ end of at least one strand of a DNA duplex. In some embodiments, a UMI is attached to both 5′ and both 3′ ends of a DNA duplex. In some embodiments, UMIs attached to a DNA duplex are identical to each other, but unique to a DNA duplex. In some embodiments, UMIs of a DNA duplex are unique to each other and unique to a DNA duplex. In some embodiments, UMIs are not unique to the DNA duplex, but when evaluated in combination with the start and/or stop sequencing sites, are unique to the DNA duplex. In some embodiments, UMIs are between about 1 nucleotide and about 20 nucleotides in length. In some embodiments, UMIs are between about 3 nucleotide and about 18 nucleotides in length. In some embodiments, UMIs are between about 5 nucleotide and about 16 nucleotides in length. In some embodiments, UMIs are between about 6 nucleotide and about 15 nucleotides in length. In some embodiments, UMIs are between about 8 nucleotide and about 15 nucleotides in length. In some embodiments, UMIs are attached to the DNA duplex by ligation. One of the benefits and features of duplex sequencing is that the association between UMI sequences added to top and bottom strand are known (e.g., are complementary to one another, or provide indication of which sequence comes from top and bottom strand) so reads from each strand can be paired back to the same original DNA duplex. This knowledge is a key component of duplex sequencing. In some embodiments, after the UMIs are unique to each duplex. In some other embodiments, there will be DNA duplexes which will share the same UMI sequence. However, the odds that two DNA duplexes will share the same UMI and the same start and stop position in the genome is highly unlikely. With this principle in mind, the sequencing reads can be de-duplicated.

After UMI attachment (e.g., an adapter comprising a UMI), a DNA duplex is amplified to produce amplified duplexes (i.e., a sequencing library, which may be defined as a collection of DNA fragments which have adapters added to facilitate their amplification and sequencing). Any suitable method known to the skilled artisan may be employed, but generally amplification is accomplished by means of polymerase chain reaction (PCR). PCR has been known in the field for a number of decades and is well-documented are the methods and protocols are readily available and will be immediately appreciated by the skilled artisan. In some embodiments, a DNA duplex is amplified by PCR.

Once amplified, an amplified DNA duplex (i.e., the sequencing library) will need to be prepared for capture by the allele-specific probes of the disclosure. In some embodiments, an amplified DNA duplex (i.e., the sequencing library) will be denatured to separate the strands of a DNA duplex, producing single-stranded amplified DNA. Any method suitable as determined by the skilled artisan may be used to denature or separate the strands, for example, without limitation, changing the temperature of the environment of a DNA duplex (e.g., apply heat, reduce temperature), sodium hydroxide (NaOH) treatments, or placing a DNA duplex in a salt rich environment. In some embodiments, a DNA duplex is denatured (e.g., strands separated) by changing the temperature of the environment. In some embodiments, the temperature change is accomplished through the application of heat.

At this point, the pool of DNA duplexes been fragmented, had UMIs attached, amplified, and denatured. Methods of the present disclosure now enrich the pool for target sequences (e.g., single-stranded amplified DNA harboring (e.g., containing) a specific mutation). The enrichment process may be accomplished by the use of probes. In some embodiments, a probe of the disclosure, is any of the probes as described herein or according to the methods of making a probe as disclosed herein. In some embodiments, a probe is an allele-specific probe. Further embodiments of probes are disclosed hereinbelow. In some embodiments, a probe comprises a sequence complementary to a portion of a single-stranded amplified DNA (e.g., such that it targets and anneals to that sequence (e.g., discriminately binds)), wherein the portion comprises a specific mutation, and a means by which to recover (e.g., capture) or separate the probe from extraneous material (e.g., unbound nucleic acids). For example, a probe may target a sequence as described herein, and comprise biotin. As such, the probe may be recovered exploiting the properties of biotin to bind streptavidin. Once the probes are bound to a single-stranded amplified DNA comprising a specific mutation, they are captured from a pool thus, producing an enriched sample. Through this process the sample will comprise a higher concentration of single-stranded amplified DNA comprising a specific mutation, than the original pool (e.g., is enriched for single-stranded amplified DNA comprising a specific mutation). This process of capturing (e.g., enriching for) single-stranded amplified DNA may occur once, or multiple times. In instances where capturing is performed multiple times (e.g., enriching multiple times), capture may be performed on a pool comprising the single-stranded amplified DNA and/or an enriched sample. In some embodiments, capture is performed at least one time. In some embodiments, capture is performed more than one time (e.g., 2, 3, 4, 5, 6, or more). In some embodiments, capture is performed more than 10 times. In some embodiments, capture is performed more than 10 times. In some embodiments, capture is performed more than 100 times. In some embodiments, capture is performed more than 1,000 times.

Additionally, capture may be performed using multiple probes. In some embodiments, more than one probe is used to capture single-stranded amplified DNA. In some embodiments, the multiple probes may be distinct, and target the same specific mutation. In some embodiments, more than one probe is used during capture, which probes are distinct from one another and target different specific mutations. By using different probes distinct and which target sequences comprising different (e.g., distinct) specific mutations, the methods of the disclosure can be used to capture (e.g., enrich) a pool of DNA duplexes for a set (e.g., panel, plurality) of mutations concurrently (e.g., simultaneously). Each probe may target a specific mutation (or more than one mutation), which is known to be associated with the same disorder, or distinct disorders. In some embodiments, wherein multiple probes are used, each targets a specific mutation (the same, distinct, or combination thereof) wherein all specific mutations are related or know to be associated with a single disorder (e.g., disease). In some embodiments, wherein multiple probes are used, each targets a specific mutation wherein at least one of the specific mutations is related or know to be associated with at least one disorder (e.g., disease) which is distinct from at least one disorder known to be associated with at least one other specific mutation.

In some embodiments, where more than one probe is used, each of the probes targets the same specific mutation targeted by other probes. In some embodiments, where more than one probe is used, at least one of the probes targets a specific mutation distinct from a specific mutation targeted by at least one other probe.

In some embodiments, at least 25 (e.g., 25, 26, 27, 27, 50, 100, or more) distinct probes are used (e.g., target 25 distinct specific mutations). In some embodiments, at least 50 (e.g., 50 or more) distinct probes are used (e.g., target 50 distinct specific mutations). In some embodiments, at least 100 distinct (e.g., 100 or more) probes are used (e.g., target 100 distinct specific mutations). In some embodiments, at least 500 distinct (e.g., 500 or more) probes are used (e.g., target 500 distinct specific mutations). In some embodiments, at least 1,000 (e.g., 1,000 or more) distinct probes are used (e.g., target 1,000 distinct specific mutations). In some embodiments, at least 10,000 (e.g., 10,000 or more) distinct probes are used (e.g., target 10,000 distinct specific mutations). In some embodiments, where more than one probe is used to capture more than one distinct specific mutation, the specific mutations are in non-overlapping regions of the genome of the subject from which the pool of DNA duplexes is obtained.

Once a probe has annealed a single-stranded amplified DNA and the probes have been recovered along with any bound single-stranded amplified DNA to produce an enriched sample, the sample is prepared for sequencing. In some embodiments, single-stranded DNA is sequenced by duplex sequencing methods. Duplex sequencing is a type of nucleic acid sequencing which uses the information from both strands of a duplex to generate results regarding the genomic profile of a sample, or subject from which a sample was obtained. Herein, we use the term “duplex sequencing” to also embody any sequencing method which derives high accuracy by requiring a consensus of sequences from both strands of each DNA duplex, although any suitable method of nucleic acid sequencing may be used. Duplex sequencing inherently possesses the ability to provide greater accuracy regarding the sequence of the nucleic acid, as computational analysis can resolve errors by using known properties of a duplex. For example, without limitation, the understanding that nucleobases form canonical base “pairings” when part of a duplex. This property of nucleic acids has been well-known since at least the latter half of the past century, and is readily understood and appreciated by those in the art. Accordingly, employing this knowledge, it is possible to infer and determine the predicted complementary sequence from the sequencing of one strand of a duplex. This inferred complementary sequence can then be compared with the results from the sequenced second strand of nucleic acid of the duplex. When such two strands are compared, they can confirm the sequences obtained, or highlight differences, thus pinpointing possible lesions (e.g., damaged bases) or mismatches only found on one strand, or sequencing errors or areas for further investigation. These differences may result from errant base insertions, deletions, or mutations (e.g., damaged bases). Further, the results of sequenced duplexes can further be compared to reference data further providing insight into possible mutations in the sequence. Accordingly, duplex sequencing provides for a high-accuracy method of resolving the sequence of nucleic acids, which accuracy permits greater resolution in determining the effect of differences therein (e.g., the effect of mutations in the genomic data). In some embodiments, an enriched sample is sequenced by duplex sequencing.

After sequencing, the data produced (e.g., sequencing results) may be queried by a user to identifying (e.g., determine, assessing, confirming) if a sequence containing a specific mutation is present. In some embodiments, a specific mutation is identified if a sequence is present in the sequencing results containing (e.g., comprising) a specific mutation. In some embodiments, a sequence containing a specific mutation may be the original top (e.g., sense, ‘+’) strand. In some embodiments, a sequence containing a specific mutation may be the original bottom (e.g., antisense, ‘-’) strand. In some embodiments, a specific mutation is identified if it appears or is contained in a sequence correlating to either the top or bottom strand. In some embodiments, a specific mutation is identified if it appears or is contained in both the top and bottom strand of the original DNA duplex. When a specific mutation appears in both strands, it is understood by the skilled artisan that the specific mutation is with respect to the base pairing, as such the sequencing will be different (as they are complementary), but will comprise the same specific mutation. Assessing the top and bottom strand to determine the pairings of sequences may be accomplished by exploiting the unique nature of the UMIs attached to each strand and which are unique to the duplex. After isolating the pairings, sequences may be aligned using customary tools for nucleic acid alignments (e.g., BLAST, HPC-BLAST, CS-BLAST, CUDASW++, DIAMOND, FASTA, etc.). Such methods are well-known in the art and software to perform such alignments is readily available for free use.

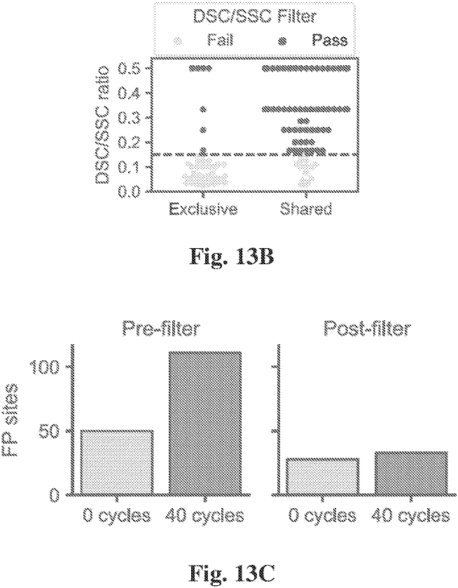

In some embodiments, the double-strand consensus (DSC) to single-strand consensus (SSC) is used to form a ratio. Methods for determining a consensus sequence are well known in the art, and in the context of nucleic acids is generally known to refer to the determination of an accepted sequence based on the most frequent nucleotide found at a given location in a sequence by comparing the position of a multitude of sequences subsequent to alignment. When establishing a DSC to SSC ratio, a consensus sequence is prepared each sequence targeted by a given probe. Optimally, there will be one given consensus sequence for each set of single-stranded amplified DNA captured by a given probe, and further yet, one given consensus sequence for the complementary strand of a single-stranded amplified DNA captured by a given probe. As mentioned elsewhere in this disclosure, the strands of single-stranded amplified DNA comprise UMIs which allow for the tracing of strands to their DNA duplex allowing for analysis of the two strands as one duplex. By exploiting this property, a consensus sequence can be established for the duplex (e.g., a double-stranded consensus sequence (DSC)). Optimally, there will only be one DSC for each set of SSCs captured by probes for a given specific mutation. Thus, an optimal DSC to SSC ratio is 0.5 (e.g., 1 DSC to 2 SSCs). However, due to imperfect capture, as well as other point mutations, sequencing errors, or errors introduced into a sequence during PCR, variations may arise in the single-stranded amplified DNA. Thus, it is improbable, if not impossible, to achieve a DSC to SSC ratio of 0.5. However, by placing a threshold on the DSC to SSC ratio, a filter is created to eliminate detection of errors which lack accuracy and/or have excess variant sequences present (e.g., FIGS. 13A-13B). In some embodiments, the DSC to SSC ration of any of the methods of the disclosure is at least 0.1 (e.g., 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, or more). In some embodiments, the DSC to SSC ratio of any of the methods of the disclosure is greater than or equal to 0.15. In some embodiments, the DSC to SSC ratio of any of the methods of the disclosure is greater than or equal to 0.2. In some embodiments, the DSC to SSC ratio of any of the methods of the disclosure is greater than or equal to 0.3.

In some embodiments, a method of the disclosure relates to methods of detecting specific mutations, wherein a specific mutation is a single nucleotide polymorphism. In some embodiments, a method of the disclosure relates to methods of detecting specific mutations, wherein a specific mutation is a structural variant.

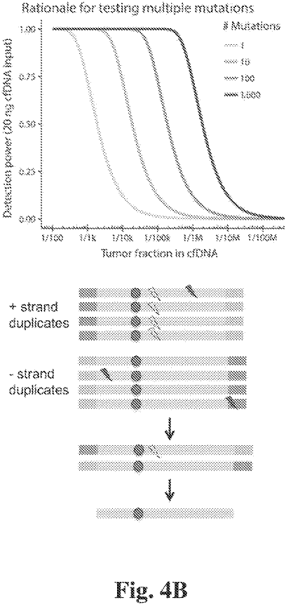

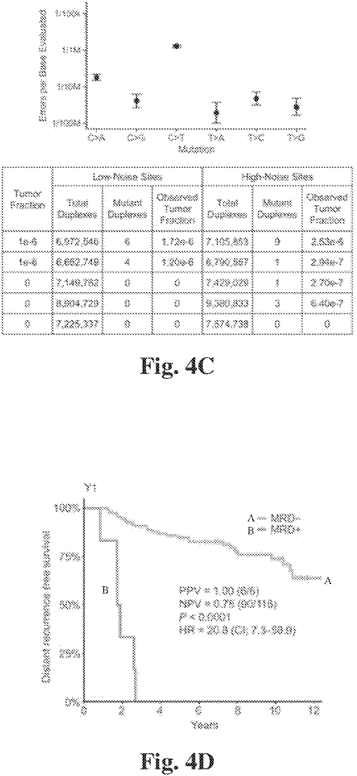

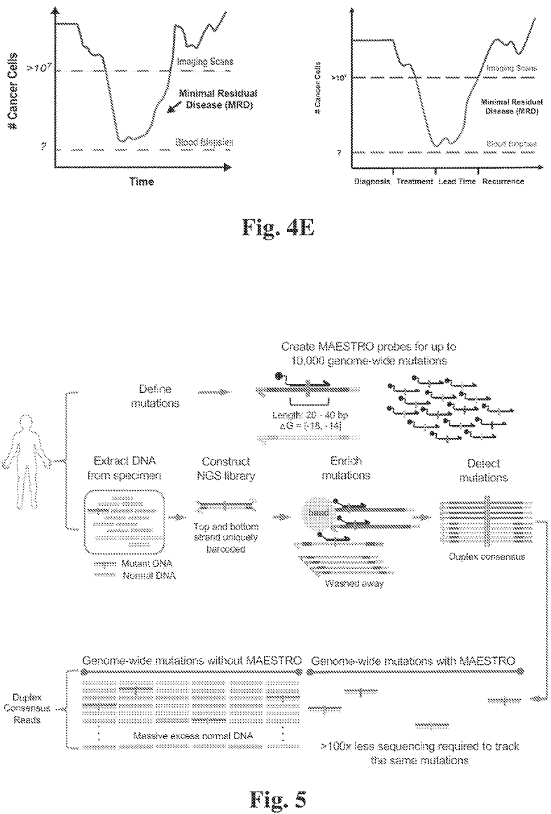

It was observed that certain bases and/or base pairings may be more prone to error (e.g., high-noise) than other bases and/or base pairings (e.g., low-noise) (FIG. 4D). By to investigate the presence of low-noise mutations (e.g., those less prone to error), the likelihood that an observed specific mutation is accurate is increased. Accordingly, when establishing the specific mutations to identify using the methods of the disclosure, those comprising specific mutations at adenine (A) and/or thymine (T) sites in a reference sequence, the confidence the mutation is accurate is increased. As used herein, a site in a reference sequence refers to the location of a base pairing in a consensus sequence for a given genome (or fragment thereof). In some embodiments, methods involve tracking low-noise mutations. In other embodiments, methods involve tracking high-noise mutations. In some embodiments, low-noise mutations comprise mutations at references sites comprising A/T base pairings. In some embodiments, high-noise mutations comprise mutations at references sites comprising cytosine.

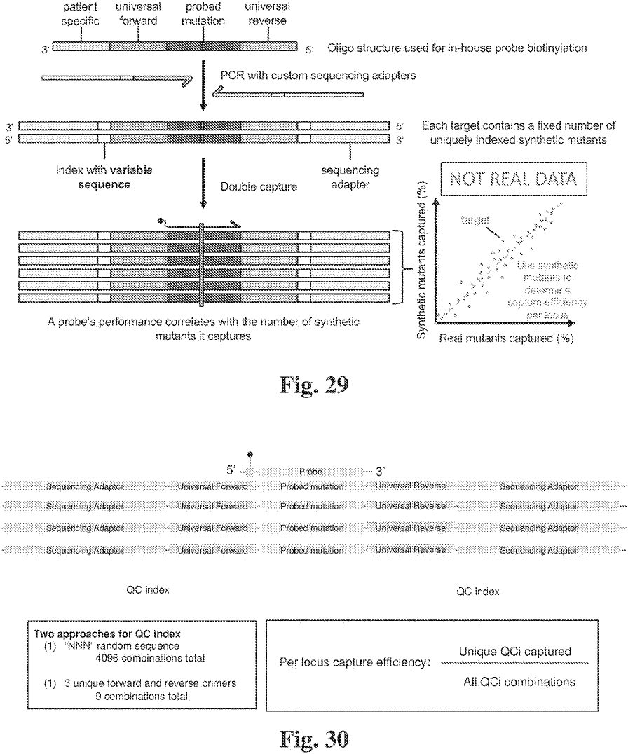

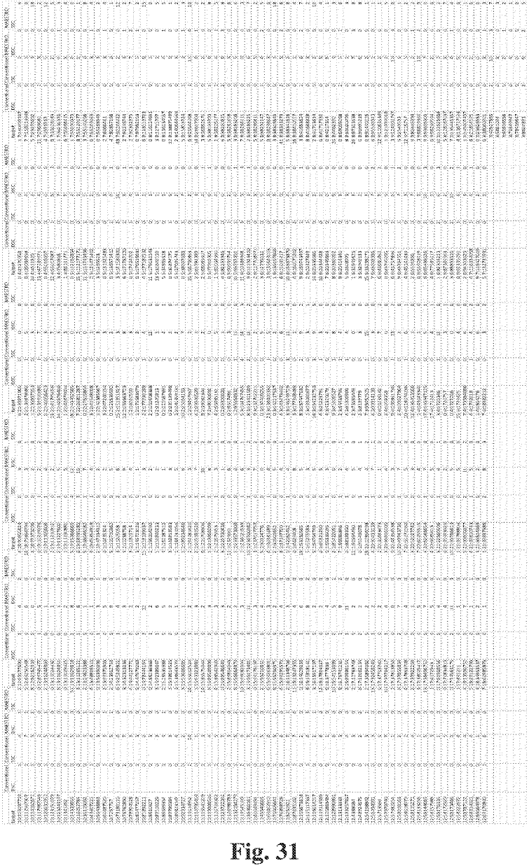

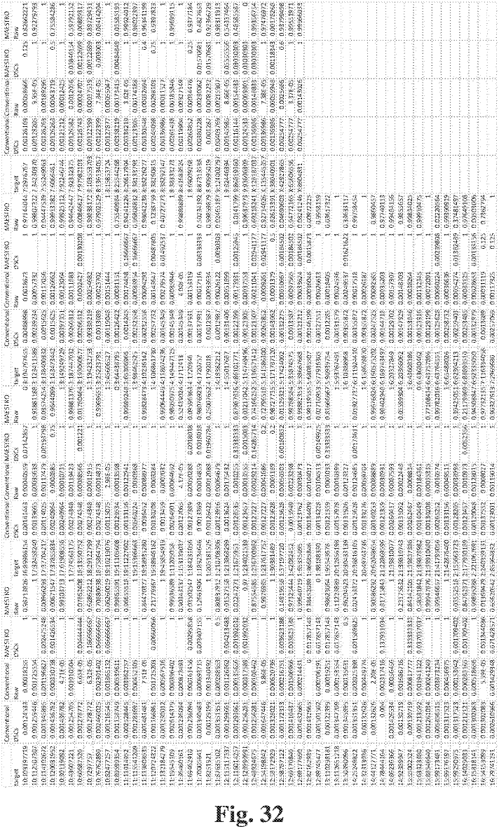

Additional steps may also be included in methods of the disclosure. For example, without limitation, a method may comprise steps to introduce controls (e.g., positive controls, controls to evaluate and/or gauge the efficiency of the method and/or the probes). In some embodiments, methods of the disclosure comprise controls. In some embodiments, a control is a positive control. As used herein, a positive control refers to creating a set of conditions in the method which is known to produce a certain result. For example, the inclusion of synthetic mutant sequences (e.g., synthetic polynucleotides) which contain a target sequence of a probe (e.g., comprise a sequencing containing a specific mutation, and which anneals to a probe). In some embodiments, methods of the disclosure comprise a positive control. In some embodiments, a positive control comprises a polynucleotide comprising a specific mutation in a sequence which anneals to a specific probe. In some embodiments, an internal control polynucleotide further comprises an index sequence. In some embodiments, the index sequence is variable. In some embodiments, an internal control polynucleotide is further flanked on the 5′ end by a universal forward binding primer and on the 3′ end by a universal reverse binding primer (e.g., FIGS. 29-30). In some embodiments, an internal control polynucleotide is further flanked on the 5′ end and the 3′ end by sequencing adapters (e.g., FIGS. 29-30). In some embodiments, an internal control polynucleotide is further flanked on the 5′ end by a universal forward binding primer and on the 3′ end by a universal reverse binding primer, which binding primers are further flanked at the distal ends (e.g., 5′ and 3′ end of the construct) by sequencing adapters (e.g., FIGS. 29-30). By using such polynucleotides, with indexes and appropriate binding primers and sequencing adapters (cumulatively a synthetic mutant) a control can be established by including the synthetic mutant in pool of DNA duplexes and/or enriched sample prior to probe capture. If a probe does not capture the synthetic mutant targeted by the probe, problems may be indicated in the method and/or conditions. If the synthetic mutant is captured, but no single-stranded amplified DNA are captured, the positive control serves to validate a method and the absence of such single-stranded amplified DNA. Use of the index of the synthetic mutant allows for tracking of multiple synthetic mutants against multiple probes (e.g., for multiple target sequences comprising specific mutations). In some embodiments, a distinct synthetic mutant is used for each distinct probe and/or distinct specific mutation.

In some embodiments, internal controls comprise a fixed number, but more than one, of synthetic mutants for a single probe (e.g., single specific mutation), wherein each synthetic mutant comprises a unique index. By using more than one, but of a known number, synthetic mutant for a given specific mutation (e.g., target sequence), each with a unique index, a method can evaluate (e.g., assess, quantify) the capture efficiency of a probe (e.g., FIGS. 29-30). For example, the number of uniquely synthetic mutants captured can be assessed against the number of specific mutations (e.g., real mutants) captured by the probes (e.g., FIGS. 29-30). This property can be used for each specific mutation of a method (e.g., for multiple, more than one). In some embodiments, a set of internal controls is used for each distinct probe, wherein each set of synthetic mutants is targeted by a probe for a specific mutation, comprises a known fixed number, and comprises a unique index.

In some embodiments, the term internal is used to describe the property that these controls are placed in the pool of DNA duplexes and/or enriched sample and are sequenced with the single-stranded amplified DNA (e.g., internal controls). The term internal controls shall be understood to include all of the aforementioned control types and variations.

In some embodiments, a specific mutation can be identified or duplex selected with at least 10 times (e.g., 10^1, 10^2, 10^3, 10^4, 10^5, 10^6) fewer sequencing reads as compared with conventional duplex sequencing methods using the methods of the disclosure. In some embodiments, a specific mutation can be identified or duplex selected with at least 50 times fewer sequencing reads as compared with conventional duplex sequencing methods using the methods of the disclosure. In some embodiments, a specific mutation can be identified or duplex selected with at least 100 times fewer sequencing reads as compared with conventional duplex sequencing methods using the methods of the disclosure. In some embodiments, a specific mutation can be identified or duplex selected with at least 500 times fewer sequencing reads as compared with conventional duplex sequencing methods using the methods of the disclosure. In some embodiments, a specific mutation can be identified or duplex selected with at least 1,000 times fewer sequencing reads as compared with conventional duplex sequencing methods using the methods of the disclosure. In some embodiments, a specific mutation can be identified or duplex selected with at least 10,000 times fewer sequencing reads as compared with conventional duplex sequencing methods using the methods of the disclosure. In some embodiments, a specific mutation can be identified, or duplex selected with at least 100,000 times fewer sequencing reads as compared with conventional duplex sequencing methods using the methods of the disclosure.

### Probes

As discussed elsewhere herein, the probes of the instant disclosure are helpful in identifying specific mutations (and/or low-abundance mutations) in pools of DNA duplexes and/or enriched samples, as each has been described herein and as derived from subjects.

In some embodiments, the probe of any of the methods of the disclosure is 10-60 nucleotides long (e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 nucleotides long). In some embodiments, the probe of any of the methods of the disclosure is about 15 to about 50 nucleotides long. In some embodiments, the probe of any of the methods of the disclosure is about 20 to about 40 nucleotides long. In some embodiments, the probe of any of the methods of the disclosure is about 12 to about 32 nucleotides long. In some embodiments, the probe of any of the methods of the disclosure is about 28 to about 32 nucleotides long. In some embodiments, the probe of any of the methods of the disclosure is 30 nucleotides long.

The probes of the disclosure can be of any configuration known in the art. For example, without limitation, the probes may comprise nucleotides of deoxyribose (e.g., DNA) and/or ribose (e.g., RNA). In some embodiments, a probe comprises DNA. In some embodiments, at least one nucleotide of the probe comprises a modification (e.g., an alteration or change to at least one component of the nucleotide (e.g., nucleobase, sugar, or phosphate group). In some embodiments, a probe contains no modified nucleotides.

In some embodiments, the probes comprise an additional moiety. A moiety may be a marker or tag. A “marker” or “tag” as used herein, refers to a molecule (e.g., nucleic acid, protein, etc.) which can be used to identify the probe in vitro and/or in vivo. Markers or tags may be any composition or molecule (e.g., nucleic acid, amino acid, peptide (e.g., glycosylated proteins, oxine, fluorescent proteins (e.g., green and/or red fluorescent protein), structures (e.g., tetracysteine loops, epitopes), any of which may be natural or synthetic (e.g., synthetic nucleic acids, amino acids, peptides, etc.))) which may be detected in vivo, in vitro, ex vivo, visually, or by exploitation of a property of the tag (e.g., fluorescence, magnetism, radioactivity, size, affinity, enzyme activity, etc.). A moiety may further be used to recover or isolate the probe, and by extension, any molecules bound thereto. In some embodiments, a moiety is a recovery moiety, wherein the moiety has a property which can be isolated and/or manipulated to separate the probe based on such property. For example, without limitation, the moiety may comprise a magnetic, chemical, physical, or affinity property which may be useful in separating the probe from extraneous material not possessing this property. Examples of such moieties are well-known in the art and any such moieties suitable may be used herein. For example, without limitation, a recovery moiety may comprise biotin. In some embodiments, an additional moiety is attached to the probe through the 5′ nucleotide. In some embodiments, a recovery moiety is attached to the probe through the 5′ nucleotide. In some embodiments, attachment is via a covalent bond.

In some embodiments, a probe comprises a nucleic acid sequence which is specific to (e.g., targets for binding) a target sequence. In some embodiments, a target sequence is representative of a specific mutation (e.g., a sequence of nucleotides equivalent to a reference sequence, but for comprising a mutation). In other words, the probe is designed to target a complementary sequence, wherein that complementary sequence comprises a specific mutation as compared to a reference sequence. In some embodiments, a specific mutation is associated or related to a disorder. Accordingly, if the probe binds this target sequence (e.g., comprising the specific mutation) it is indicative of the presence of the nucleic acid data associated with the disorder.

In some embodiments, the sequence portion of the probe which binds the specific mutation, target sequence, or SNP is located within the middle 50% of nucleotides comprising the probe, or in other words, the portion of the probe comprising the nucleotides not in the first quarter of nucleotides of the probe (e.g., the quarter comprising the 5′ end), or last quarter of nucleotides of the probe (e.g., the quarter comprising the 3′ end). In some embodiments, the sequence portion of the probe which binds the specific mutation, target sequence, or SNP is located within the middle third of nucleotides comprising the probe, or in other words, the portion of the probe comprising the nucleotides not in the first third of nucleotides of the probe (e.g., the third comprising the 5′ end), or last third of nucleotides of the probe (e.g., the third comprising the 3′ end).

In some embodiments, the nucleotide of the probe which binds the specific mutation or SNP, is located within the middle 50% of nucleotides comprising the probe, or in other words, the portion of the probe comprising the nucleotides not in the first quarter of nucleotides of the probe (e.g., the quarter comprising the 5′ end), or last quarter of nucleotides of the probe (e.g., the quarter comprising the 3′ end). In some embodiments, the nucleotide of the probe which binds the specific mutation or SNP is located within the middle third of nucleotides comprising the probe, or in other words, the portion of the probe comprising the nucleotides not in the first third of nucleotides of the probe (e.g., the third comprising the 5′ end), or last third of nucleotides of the probe (e.g., the third comprising the 3′ end). In some embodiments, the nucleotide of the probe which binds the specific mutation or SNP is located within the middle 6% of nucleotides comprising the probe, or in other words, the portion of the probe comprising the nucleotides not in the first 47% of nucleotides of the probe, or last 47% of nucleotides of the probe (e.g., the third comprising the 3′ end).

In wherein an allele-specific probe is evaluated, and/or modified to increase/decrease, the Gibbs free energy (ΔG) of the allele-specific probe annealing to its complementary sequence. By controlling and/or modifying this property of the probe, the specificity and ability for the probe to more precisely discriminate sequences and single-stranded amplified DNA, can be modulated (e.g., increased, decreased). Further, by controlling this property, the stability of bound probes can also be modulated (e.g., increase, decreased). In some embodiments, the Gibbs free energy (ΔG) of an allele-specific probe annealing to its complementary sequence is at least -25 Kcal/mol at Temp =50° C., but no more than -5 kcal/mol at Temp =50° C. (e.g., -25, -24, -23, -22, -21, -20, -19, -18, -17, -16, -15, -14, -13, -12, -11, -10, -9, -8, -7, -6, -5J, or increment therein). In some embodiments, the Gibbs free energy (ΔG) of an allele-specific probe annealing to its complementary sequence is at least -23 Kcal/mol at Temp =50° C., but no more than -7 kcal/mol at Temp =50° C. In some embodiments, the Gibbs free energy (ΔG) of an allele-specific probe annealing to its complementary sequence is at least -21 kcal/mol at Temp =50° C., but no more than -9 kcal/mol at Temp =50° C. In some embodiments, the Gibbs free energy (ΔG) of an allele-specific probe annealing to its complementary sequence is at least -20 kcal/mol at Temp =50° C., but no more than -12 kcal/mol at Temp =50° C. In some embodiments, the Gibbs free energy (ΔG) of an allele-specific probe annealing to its complementary sequence is at least -19 kcal/mol at Temp =50° C., but no more than -13 kcal/mol at Temp =50° C. In some embodiments, the Gibbs free energy (AG) of an allele-specific probe annealing to its complementary sequence is at least -18 kcal/mol at Temp =50° C., but no more than -14 kcal/mol at Temp =50° C. In some embodiments, the Gibbs free energy (AG) of an allele-specific probe annealing to its complementary sequence is at least -17 kcal/mol at Temp =50° C., but no more than -15 kcal/mol at Temp =50° C. In some embodiments, the Gibbs free energy (AG) is modified by adjusting the length of the sequence of the probe which will bind a target sequence (e.g., comprising a specific mutation). In some embodiments, length is increased. In some embodiments, length is decreased. In some embodiments, the length is adjusted iteratively until the Gibbs free energy (AG) is within the ranges preferred. In some embodiments, the length is adjusted iteratively until the Gibbs free energy (ΔG) is within the ranges as described herein.

A further evaluation and design consideration given to constructing a probe according to the present disclosure comprises evaluating the likely ability of the probe to bind other portions of a nucleic acid (e.g., other areas, portions, fragments, of a genome). Accordingly, once a probe sequence is developed, it may be evaluated to see if it is homologous with any other areas of a genome of a subject from which the pool of DNA duplexes and/or enriched sample was taken. There are a multitude of well-known methods, tools, and software programs publicly, and freely available to perform such searches (e.g., BLAST, etc.). In some embodiments, a target sequence of the allele-specific probe is homologous with less than 20 sequences of a reference genome of the subject. In some embodiments, a target sequence of the allele-specific probe is homologous with less than 15 sequences of a reference genome of the subject. In some embodiments, a target sequence of the allele-specific probe is homologous with less than 10 sequences of a reference genome of the subject. In some embodiments, a target sequence of the allele-specific probe is homologous with less than 5 sequences of a reference genome of the subject. In some embodiments, a target sequence of the allele-specific probe is 100% homologous with less than 20 sequences of a reference genome of the subject. In some embodiments, a target sequence of the allele-specific probe is 100% homologous with less than 15 sequences of a reference genome of the subject. In some embodiments, a target sequence of the allele-specific probe is 100% homologous with less than 10 sequences of a reference genome of the subject. In some embodiments, a target sequence of the allele-specific probe is 100% homologous with less than 5 sequences of a reference genome of the subject. If there are an excess number of sites which are homologous with the target sequence of the probe (e.g., the sequence it will bind comprising a specific mutation), a probe may be modified (e.g., altered). For example, without limitation, the sequence targeted may be frameshifted in one direction or the other relative to the position of the nucleotide(s) of the specific mutation. This modification may be performed in either direction. Further, this modification may include altering the length of the probe as well (while keeping the Gibbs free energy in an appropriate range), or the length of the probe may remain constant during this shift. In some embodiments, a sequence targeted by an allele-specific probe is moved 5 nucleotides, or less (e.g., 1, 2, 3, 4, or 5) in the 5′ direction. In some embodiments, a sequence targeted by an allele-specific probe is moved 10 nucleotides, or less (e.g., 1, 2, 3, 4, 5, 6, 7, 8, 9, or 10) in the 5′ direction. In some embodiments, a sequence targeted by an allele-specific probe is moved 5 nucleotides, or less (e.g., 1, 2, 3, 4, or 5) in the 3′ direction. In some embodiments, a sequence targeted by an allele-specific probe is moved 10 nucleotides, or less (e.g., 1, 2, 3, 4, 5, 6, 7, 8, 9, or 10) in the 3′ direction.

In some embodiments, a probe is designed and/or selected for use according to one or methods of the present disclosure, due at least in part to its annealing temperature. For example, without limitation, in some embodiments, an allele-specific probe has an annealing temperature of at least 44 degrees Celsius (°C.), but no more than 56° C. In some embodiments, an allele-specific probe has an annealing temperature of at least 45 degrees Celsius (°C.), but no more than 55° C. In some embodiments, an allele-specific probe has an annealing temperature of at least 47 degrees Celsius (°C.), but no more than 54° C. In some embodiments, an allele-specific probe has an annealing temperature of at least 48 degrees Celsius (°C.), but no more than 52° C. In some embodiments, an allele-specific probe has an annealing temperature of at least 49 degrees Celsius (°C.), but no more than 51° C. In some embodiments, an allele-specific probe has an annealing temperature of at least 50 degrees Celsius (°C.). In still other embodiments, the allele-specific probe has an annealing temperature of at least 40° C., or at least 41° C., of at least 42° C., of at least 43° C., of at least 44° C., of at least 45° C., of at least 46° C., of at least 47° C., of at least 48° C., of at least 49° C., of at least 50° C., of at least 51° C., of at least 52° C., of at least 53° C., of at least 54° C., of at least 55° C., of at least 56° C., of at least 57° C., of at least 58° C., of at least 59° C., of at least 60° C., of at least 61° C., of at least 62° C., of at least 63° C., of at least 64° C., of at least 65° C., of at least 66° C., of at least 67° C., of at least 68° C., of at least 69° C., of at least 70° C., of at least 71° C., of at least 72° C., of at least 73° C., or of at least 74° C. but not more than 75° C., or but not more than 50° C., but not more than 51° C., but not more than 52° C., but not more than 53° C., but not more than 54° C., but not more than 55° C., but not more than 56° C., but not more than 57° C., but not more than 58° C., but not more than 59° C., but not more than 60° C., but not more than 61° C., but not more than 62° C., but not more than 63° C., but not more than 64° C., but not more than 65° C., but not more than 66° C., but not more than 67° C., but not more than 68° C., but not more than 69° C., or not more than 70° C.

In some embodiments, a recovery moiety is attached to the 5′ end of an allele-specific probe. In some embodiments, an MGB is attached to the 3′ end of an allele-specific probe. In some embodiments, a recovery moiety is biotin. However, it should be noted that any suitable appropriate tag or moiety providing a means or property by which the probe (and any single-stranded amplified DNA bound thereto) may be separated and/or recovered may be used. Appropriate such tags and/or moieties are well-known in the art and will be readily discernable by the skilled artisan. In some embodiments, an allele-specific probe comprises biotin. In some embodiments, biotin is recovered (e.g., captured) by exploiting its ability to preferentially bind avidin. In some embodiments, biotin is recovered (e.g., captured) by exploiting its ability to preferentially bind streptavidin. In some embodiments, biotin is recovered (e.g., captured) by exploiting its ability to preferentially bind neutravidin.

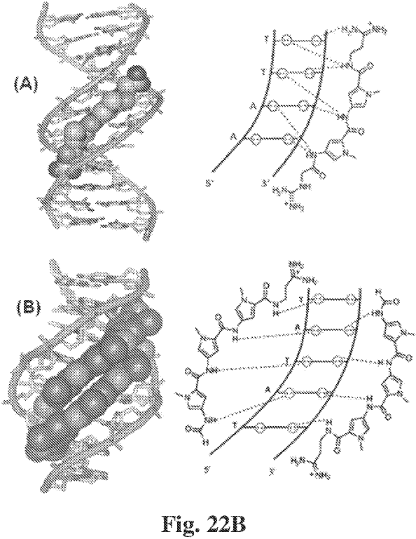

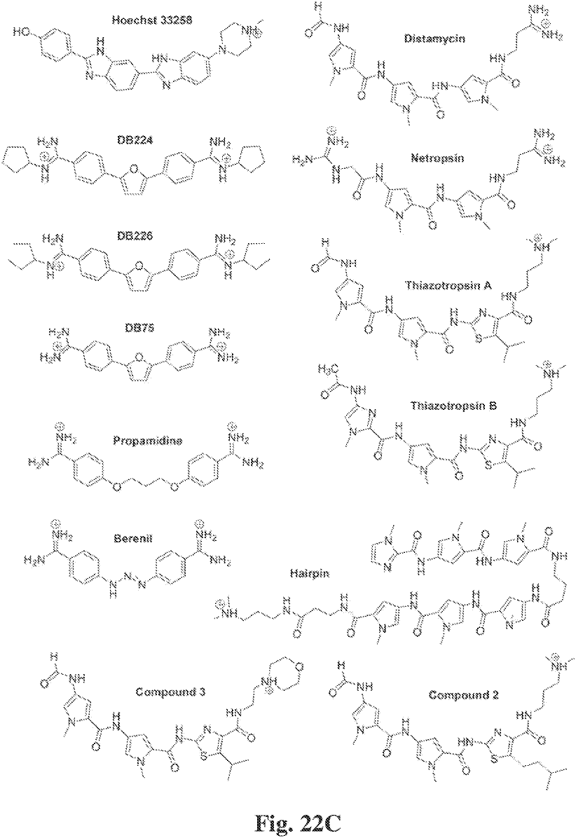

In some embodiments, the disclosure relates to an allele-specific probe, further comprising a minor grove binder (MGB). MGBs are molecules, typically crescent-shaped molecules, which selectively bind minor grooves of nucleic acids. MGBs typically bind with specific sequences and may bind non-covalently by a combination of directed hydrogen bonding to base pair edges. Examples of MGBs are shown in FIG. 22C, which bind the minor grooves of DNA (FIGS. 22A-22B). Examples of MGBs increasing discrimination of mismatches in ODNs (Oligodeoxynucleotides) as shown in FIG. 22D. The MGBs ODNs (+MGB) are shown to have a greater free energy difference (ΔΔG) in the MGB region as compared to the ODN absent the MGB (-MGB). In certain embodiments, the probes may be modified by any known means to increase the ΔΔG between match and mismatch, e.g., locked nucleic acid; peptide nucleic acid; SuperG,C,T,A (e.g., available or obtainable commercially); XNA nucleotides; etc).

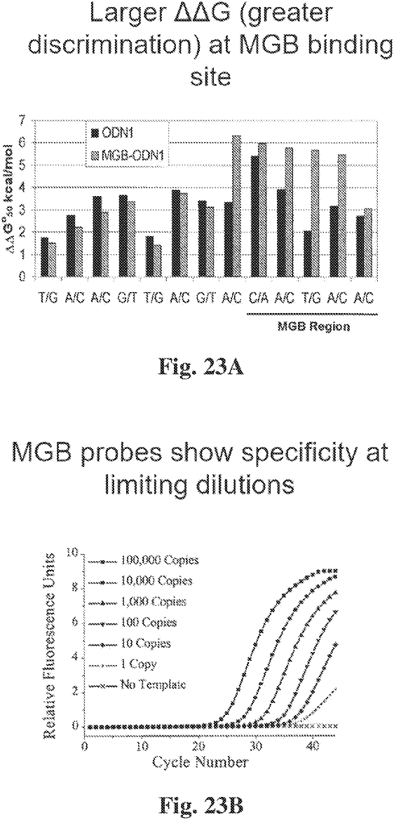

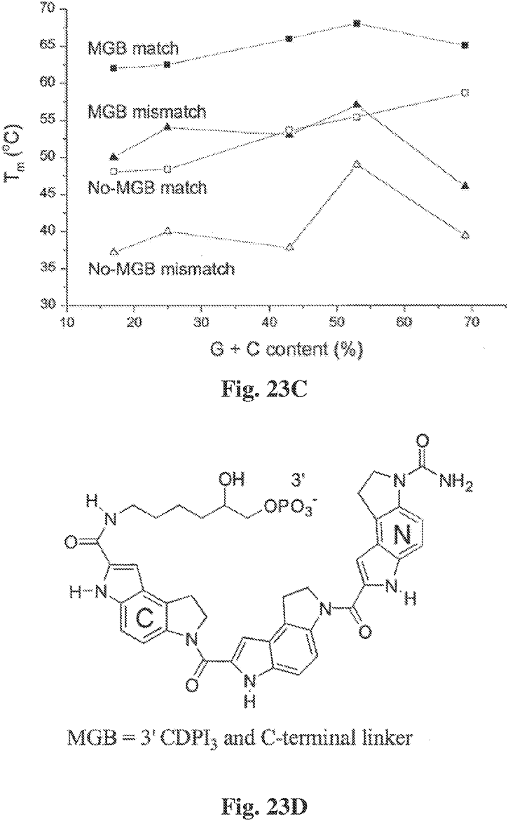

Additionally, the MGB are still effective at discriminating and binding target sequences at dilutions which are increasingly small (e.g., 1 copy) (FIG. 23B). Finally, MGBs are shown to increase the melting temperature (Tₘ) of bound ODN to in various configurations, Mismatches±, MGB±, wherein ODNs with no mismatches and MGBs show an elevated Tₘ (FIG. 23C). Thus, the addition of MGBs to the probes of the disclosure will improve affinity and specificity, further improving the resolution and sensitivity of the methods herein. In some embodiments, an allele-specific probe comprises an MGB. In some embodiments, an MGB comprises at least one of the MGBs of FIG. 22C.

In some embodiments, the disclosure relates to a method of making allele-specific probes, the method comprising: for each target sequence (e.g., sequence comprising a specific mutation), a 30-nucleotide probe is created with the altered base (e.g., nucleotide targeting the specific mutation, e.g., the nucleotide complementary to the specific mutation) at its center. The probe may be designed against the plus strand or the minus strand depending on the base change. The length is adjusted until the estimated delta G of the probe sequence is within an acceptable range (yielding probe candidates between 20 and 40 nucleotides in length). This same strategy is used while shifting the probe’s center up to 5 bp in either direction to create multiple candidates for each target. A BLAST search is performed and the candidate with the highest specificity for the target is selected. A given target may be removed from the design if its probe characteristics (delta G, length, %GC, melting temperature, number of BLAST hits) do not meet pre specified requirements.

In some aspects, the disclosure relates to a method of making an allele-specific probe, the method comprising: (a) identifying a specific mutation in a nucleic acid sequence of a genome; (b) generating a complementary nucleic acid (CNA) including a complementary base to the specific mutation; and (c) attaching a recovery moiety to the 5′ nucleotide of the allele-specific probe; wherein the complementary base is in the middle 50% of nucleotides of the CNA; wherein, the CNA comprises at least 12, but no more than 60 nucleotides; wherein the Gibbs free energy of the CNA and the nucleic acid comprising the specific mutation is at least -20, but no more than -12; wherein the annealing temperature of the allele-specific probe is at least 48 degrees Celsius (°C.), but no more than 52° C.; and wherein the CNA is 100% homologous with less than 10 sequences within the genome.

These and other aspects and embodiments will be described in greater detail herein. The description of some exemplary embodiments of the disclosure are provided for illustration purposes only and not meant to be limiting. Additional compositions and methods are also embraced by this disclosure.

### Kits

In an aspect, the disclosure relates to kits for performing one or more of the methods of the disclosure (e.g., identification of specific mutations and/or low-abundance mutations) in a pool of DNA duplexes and/or enriched sample.

In some embodiments, a kit comprises materials and/or reagents to carry out one or more of the methods of the disclosure. For example, without limitation, the kit may comprise the components and/or reagents to perform the entire method, and/or any portion thereof. In some embodiments, materials and devices are provided in the kits which provide for the acquisition and/or procurement of a pool of DNA duplexes. In some embodiments, a kit comprises devices and/or housings (e.g., containers) to hold any of the liquid stages or materials of one or more methods of the disclosure.

In some embodiments, a kit comprises any of the probes as described herein useful for one or more of the methods of the disclosure.

In some embodiments, a kit comprises materials and/or reagents to carry out the method of making an allele-specific probe according to the instant disclosure. In some embodiments, a kit comprises a probe as produced by the methods of the disclosure.

In some embodiments, a kit comprises materials, devices, and/or reagents to carry out a liquid biopsy to detect one or more mutations.

Instructions for performing one or more of the methods of the disclosure may also be included in the kits described herein.

The kit may contain packaging or a container with components as described herein.

Other suitable components to include in such kits will be readily apparent to one of skill in the art, taking into consideration the desired application and use of one or more of the methods of the disclosure.

### Examples

### Introduction

The ability to assay large numbers of low-abundance mutations is crucial in biomedicine. Yet, the technical hurdles of sequencing multiple mutations at extremely high depth and accuracy remain daunting. For sequencing low-level mutations, it’s either ‘depth or breadth’ but not both. Here, it is reported, a simple and powerful approach to accurately track thousands of distinct mutations with minimal reads. Our technique called MAESTRO (minor allele enriched sequencing through recognition oligonucleotides) employs massively-parallel mutation enrichment to empower duplex sequencing-one of the most accurate methods-to track up to 10,000 low-frequency mutations with up to 100-fold less sequencing. In example use cases, show that MAESTRO could enable mutation validation from cancer genome sequencing studies. It is also shown that the method could track thousands of mutations from a patient’s tumor in cell-free DNA, which may improve detection of minimal residual disease from liquid biopsies. In all, MAESTRO improves the breadth, depth, accuracy, and efficiency of mutation testing. Here is shown an accurate and efficient technique to track large numbers of distinct tumor mutations, identified from patients’ tumor biopsies, in cfDNA.

Mutations in DNA emerge from single cells, define cell populations, and establish genetic diversity. Considering the vast genetic diversity of living organisms and the significance of mutations in disease biology, there is a growing need to assay many distinct, low-abundance mutations in multiple areas of biomedicine spanning oncology, obstetrics, transplantation, infectious disease, genetics, microbiomics, forensics, and beyond. Yet, the intrinsic tradeoff in breadth-versus-depth of DNA sequencing means that either few mutations can be assayed at high depth, or many mutations at low depth-not both. High depth (i.e. many reads per genomic locus) is required to accurately detect low-abundance mutations, but this severely limits breadth (i.e. number of distinct loci). This explains why, despite massive reductions in sequencing costs, it remains prohibitively expensive to test for large numbers of distinct, low-abundance mutations.

Duplex sequencing is one of the most accurate methods for mutation detection, with 1000-fold fewer errors than standard sequencing, but adds significant cost. By requiring mutations to be present in replicate reads from both strands of each DNA duplex, many of the errors in sample preparation and sequencing can be overcome to enable reliable detection of low-abundance mutations. Yet, up to 100-fold more reads per locus are required-a challenge that is exacerbated when tracking many low-abundance mutations. Less stringent methods exist that require fewer reads, but compromising specificity to save cost would be deeply problematic for applications that impact patient care. While methods to enrich rare mutations have been developed, none have employed high-accuracy sequencing, nor tracked many rare mutations

Liquid biopsy represents an application for which accurate, low-cost tracking of many distinct mutations could empower clinical decisions. For instance, applying liquid biopsies to detect minimal residual disease (MRD) after cancer treatment has the potential to inform whether surgery is needed after neoadjuvant therapy, whether adjuvant therapy is needed after surgery, and ultimately, whether it is safe to stop treatment. It could also enable treatment response to be monitored over several log-fold-changes in cancer burden, which has been critical in hematologic malignancies, but is not yet feasible for most patients due to limited sensitivity.

One promising way to improve sensitivity of liquid biopsies is to track many patient-specific tumor mutations in cell-free DNA (cfDNA), recognizing that not all mutations may be present in an individual blood tube when tumor DNA in the bloodstream is sparse (i.e. less than a genome equivalent of tumor DNA per tube). Yet, this has been challenging, because to rely upon any subset for MRD detection requires extremely accurate sequencing of many rare mutations. It is reasoned that methods to deplete the normal (i.e. non-tumor derived) cfDNA could enable accurate, low-cost tracking of thousands of mutations in a patient’s tumor genome and improve MRD detection.

Here, is described ‘MAESTRO’ (minor allele enriched sequencing through recognition oligonucleotides), a technique which combines massively-parallel mutation enrichment with duplex sequencing to enable accurate, low-cost mutation testing. In contrast to conventional hybrid-capture duplex sequencing (herein referred to as ‘Conventional’), which uses long probes to capture mutant and wild type with similar efficiency, MAESTRO uses short probes to enrich for patient-specific mutant alleles and uncovers the same mutant duplexes using up to 100-fold fewer reads. The performance of MAESTRO is first established in dilution series. Then, two proof-of-principle applications are provided. In the first, it is shown that MAESTRO could enable verification of low-abundance mutations discovered from cancer whole-exome sequencing. In the second, it is shown that MAESTRO could enable thousands of mutations from a patient’s tumor to be assayed in cfDNA, which may improve the detection of MRD.

### Methods

### Patients and Samples

All patients provided written informed consent to allow the collection of blood and/or tumor tissue and analysis of clinical and genetic data for research purposes. Patients with triple-negative breast cancer (TNBC) and a tumor size greater than 1.5 centimeters (>1.5 cm) were prospectively identified for enrollment into tissue analysis and banking cohorts (Dana-Farber Cancer Institute [DFCI] IRB-approved protocol 07130). Patients had plasma isolated from 20 cubic centimeters (cc) blood in Ethylenediaminetetraacetic acid (EDTA) tubes and tissue sampling performed within six months of diagnosis. All patients completed the following course of neoadjuvant Phase II therapy: Bevacizumab x 1 dose; Doxorubicin/Cyclophosphamide x 4 cycles plus Bevacizumab; Paclitaxel x 4 cycles plus Bevacizumab. Blood draws were taken before each course. A Residual Cancer Burden (RCB) score was calculated after surgery. For those patients with sufficient tumor tissue, exome-sequencing identified mutations captured using a Conventional assay.³⁴ From within this cohort four TNBC patients were identified who had tested MRD-negative using the exome-wide panel but who experienced metastatic recurrence. For these patients, MAESTRO was applied to analyze genome-wide tumor mutations. HapMap DNA from NA12878 and NA19238 was purchased from Corielle. This research was conducted in accordance with the provisions of the Declaration of Helsinki and the U.S. Common Rule.

### Defining Mutations to Track

For the HapMap panels, VCF files were taken from the Genome in a Bottle Consortium49 (NA12878) and 1000 Genomes project50 (NA19238). Sites specific to NA12878 were subsampled to create MAF files and were subsequently run through probe design to create the 438 and 10,000 SNV (single nucleotide variant) fingerprints. Tumor DNA was extracted from fresh-frozen tumor samples. All patients’ tumor DNA underwent whole-exome sequencing to identify trackable mutations for conventional capture. Of the four patients selected for MAESTRO, tumor DNA underwent PCR-free whole-genome sequencing. Illumina output from whole-genome sequencing was processed by the Broad Picard pipeline and aligned to hg19 using BWA. The GATK best practices workflow was used on the Terra platform to detect somatic SNVs and indels in the deep whole-genome sequencing data using tumor/normal calling (see Terra workflow). Somatic mutation calls were subset to only SNVs and passed the candidate SNVs for tracking to the probe design pipeline. By sequencing each patient’s tumor and normal to adequate depth is was possible to avoid tracking variants arising from clonal hematopoiesis.

### Probe Design

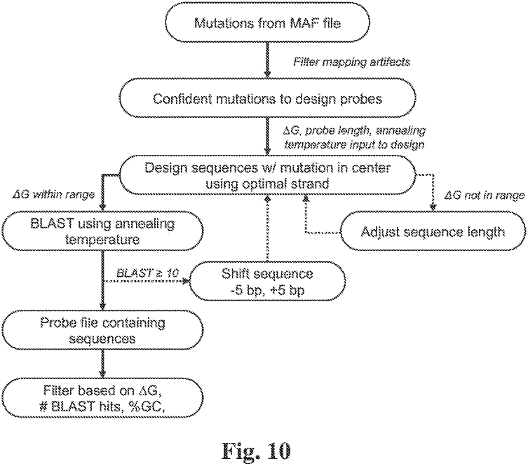

Mutations in MAF (mutation annotation format) were first checked for specificity in the reference genome to filter out potential mapping artifacts. The resulting filtered MAF was then used as input into probe design. Conventional probe design was performed on the filtered MAF as previously described 34. For MAESTRO probe design, along with the mutation file, initial probe length (default = 30 bp), annealing temperature (default = 50° C.), and AG range (default = -18 to -14 kcal/mol) were used as input. For AG and melting temperature calculations, annealing temperature was used, [Na+] = 50 mM, [Mg2+] = 0 mM, and [DNA] = 250 nM. An initial sequence was designed for the given length with the mutation at its center. If the sequence was within the specified AG range, it proceeded through the subsequent design steps, otherwise the sequence length was adjusted until it fell within the range. A modified BLAST was performed where the melting temperature for each hit was calculated and if it was less than the annealing temperature, it was removed. If there were 10 or greater pass-filter BLAST hits, the sequence was redesigned using a sliding window (e.g., shifted forward bases or backward bases). This resulted in the mutation being offset from the center of the sequence, but still provided good enrichment. The sequence with the minimum BLAST hits was then chosen. All sequences were output in a tab-delimited file, and the results were filtered based on length, GC content, AG, and the number of BLAST hits before ending up with the final panel design (FIG. 10).

### In-House Biotinylation of Probe Panel

Patient-specific oligo pools ordered from Twist Bioscience contained universal forward and reverse primer binding sites. Amplification of the oligo pool was performed using an internally biotin-modified forward primer containing a dU base directly 5′ to the biotinylated dT and an unmodified reverse primer containing a BciVI recognition sequence at its 3′ end. The PCR product was purified using Zymo’s DNA Clean & Concentrator-25 columns. Two micrograms of biotinylated, double-stranded product were sequentially subject to the following 100 µL one-tube enzymatic reaction: 40 units BciVI for 60 minutes at 37° C.; 10 units Lambda Exonuclease for 30 minutes at 37° C. followed by 20 minutes at 80° C.; 7 units USER Enzyme for 30 minutes at 37° C. (NEB).⁵¹ Zymo’s Oligo Clean & Concentrator columns were used to purify short, single-stranded, biotinylated probes for hybrid capture.

### DNA Extraction and Library Construction

Healthy gDNA from two HapMap cell lines, NA12878 and NA19238, were sheared to 150 bp fragments using a Covaris E220/LE220 Ultrasonicator. Sheared DNA was quantified using the Quant-iT Picogreen dsDNA assay kit on a Hamilton STAR-line liquid handler. Tumor fraction dilutions were created by spiking sheared gDNA from NA12878 (“tumor”) into NA19238 (“normal”) at 0, 1:1K, 1:10K, 1:100K, and 1:1M tumor fractions. All libraries were constructed with 20 ng sheared gDNA using the Kapa Hyper Prep Kit with custom dual-index duplex UMI adapters (IDT). These UMI adapters allowed tracking of the top and bottom strand of each unique starting molecule despite rounds of amplification. Processing of patient blood samples followed the same protocol as previously described.⁵⁶ Germline DNA (gDNA) was extracted from either buffy coat or whole blood using the QIAsymphony DSP DNA Mini kit and sheared. Cell-free DNA (cfDNA) was extracted from plasma using the QIAsymphony DSP Circulating DNA Kit. cfDNA and gDNA libraries were constructed in the same manner as HapMap DNA. In cases where there was insufficient library remaining for a subsequent capture, 200 ng of library was subject to additional rounds of PCR to generate workable mass (>1 µg) for hybrid capture using KAPA’s library amplification primer mix. In cases where technical replicates of the same library were needed, libraries were reindexed using a new set of P5/P7 indices (IDT).

### MAESTRO Capture

Hybrid capture using biotinylated, short probe panels was performed using xGen Hybridization and Wash Kit with xGen Universal Blockers (IDT) using a protocol adapted from Schmitt, et al.⁵⁷ Each hybrid capture contained 1 µg of library and 0.75 pmol/µL of MAESTRO probes (IDT or Twist Bioscience), using wells in the middle of the 96-well plate to prevent temperature fluctuations. The hybridization program began at 95° C. for 30 seconds. This was followed by a stepwise decrease in temperature from 65° C. to 50° C., dropping 1° C. every 48 minutes. Finally, the plate was held at 50° C. for at least four hours, making the total time in hybridization 16 hours. Heated wash buffer was kept at 50° C. (lid temp 55° C.) and heated wash steps were performed at 50° C. After the first round of hybrid capture, 16 cycles of PCR were applied. The product was subject to a second round of hybrid capture using half volumes of Cot-1 DNA, xGen Universal Blockers, and probes. This was followed by another 16 cycles of PCR. Apart from these differences, MAESTRO double capture was performed using the same protocol as outlined in Parsons, et al.⁵⁴ Final captured product was quantified and pooled for sequencing on an Illumina HiSeq 2500 (101 bp paired-end reads) or a HiSeqX (151 bp paired-end reads) with a target raw depth of 10,000 x per site.

### Conventional Capture

The following described protocol was outlined previously in Parsons, et al.⁵⁴ Hybrid capture using a panel consisting of patient-specific (i.e. germline informed), biotinylated 120 nt probes was performed using the xGen Hybridization and Wash Kit with xGen Universal Blockers (IDT). For each Conventional capture reaction, libraries were pooled up to 6-plex with 500 ng input each and 0.56 to 0.75 pmol/µL, of probe panel was applied (IDT). The hybridization program began at 95° C. for 30 seconds. This was followed by 65° C. for 16 hours. Heated wash buffer was kept at 65° C. (lid temp 70° C.) and heated wash steps were performed at 65° C. the first round of hybrid capture, 16 cycles of PCR were applied. The product was subject to a second round of hybrid capture using half volumes of Cot-1 DNA, xGen Universal Blockers, and probes. This was followed by another 8 cycles of PCR. Final captured product was quantified and pooled for sequencing on an Illumina HiSeq 2500 (101 bp paired-end reads) or a HiSeqX (151 bp pairedend reads) with a target raw depth of 1,000,000 x per site.

### Quantification of Library Conversion Efficiency by ddPCR

To quantify probe capture efficiency, a ddPCR assay was designed to target the flanking adapter regions. Only fragments with successful double ligation were exponentially amplified within the QX200 ddPCR EvaGreen Supermix (Bio-Rad). Varying DNA inputs into LC (3 ng, 10 ng, 20 ng, 50 ng) were tested for their varying conversion efficiencies and adjusted to an unligated control (Table 1).

TABLE 3

ddPCR Assay Design for Library Conversion Efficiency
Primer 1: CACTCTTTCCCTACACGACG (SEQ ID NO: 1)
Primer 2: AGTTCAGACGTGTGCTCTTC (SEQ ID NO: 2)

### Quantification of Probe Capture Efficiency by ddPCR

To quantify probe capture efficiency, a ddPCR assay was designed to target a homozygous mutation site chosen from the 438 SNV HapMap fingerprint (see ddPCR assay design below). Conventional and MAESTRO hybrid capture was performed on pure tumor Hapmap gDNA libraries, with all waste streams collected from washes. The total number of mutant molecules into hybrid capture and lost during hybrid capture were quantified by using the designed ddPCR assay.^{54,55} Probe capture efficiencies were determined using the equation below (Table 2).

TABLE 4

ddPCR Assay Design for Probe Capture Efficiency
Homozygous mutation site: g.chr2:29940529A>T
Primer 1 (targeting a consensus sequence on our duplex UMI adapters): CACTCTTTCCCTACACGACG (SEQ ID NO: 3)
Primer 2: ATGTCCAGGTCATAGCTCC (SEQ ID NO: 4)
Taqman probe targeting the tumor DNA sequence: /5HEX/-CAACAAACATGCCATCTCCTTCTCCTGA-/ZEN/31ABkFO/ (SEQ ID NO: 5)

$$\begin{matrix}C a p t u r e e f f i c i e n c y = f - \\ \frac{T h e t o t a l n u m b e r o f m u t a n t m o l e c u l e s l o s t d u r i n g h y b r i d s e l e c t i o n}{T h e t o t a l n u m b e r o f m u t a n t m o l e c u l e s i n t o h y b r i d s e l e c t i o n}\end{matrix}$$

### Sequencing and Data Analysis

Sequencing and pre-processing of BAM files followed a similar protocol as previously described 33,34 with the following changes. Before grouping reads by UMI, read groups were added to samples from the same library and samples were merged into a single BAM. This ensured identical molecules found in different samples were given the same family ID from Fgbio’s GroupReadsByUmi (Fulcrum Genomics). The resulting BAM was then pushed through GroupReadsByUmi and split afterwards by the added read group tag. The split BAMs were then passed through the consensus calling workflow. Consensus BAM files were indel realigned using GATK 4 before calling mutations using custom scripts. Noise filtering based on DSC/SSC ratio (total mutant DSCs / total mutant SSCs) was performed on all MAESTRO samples. For mutation calling in clinical samples, both matched tumor and normal were used. It was required that each mutation be seen in the tumor and not in the normal in order for the mutation to be considered. Processing of BAM files was automated using a Snakemake53 workflow.

### Miredas Minimal Residual Disease Analysis Scripts

A suite of scripts (Miredas) was used for calling mutations and creating metrics files. In the Snakemake workflow, MiredasCollectErrorMetric uses the duplex BAM file to describe the number of errors and calculates errors per base sequenced. MiredasDetectFingerprint uses the duplex BAM file to call mutations and MiredasDetectFingerprintSsc uses the single-stranded BAM file to call mutations. This single-stranded output of MiredasDetectFingerprintSsc is used along with the duplex MiredasDetectFingerprint output to create DSC/SSC ratios.

### VAF/Recall

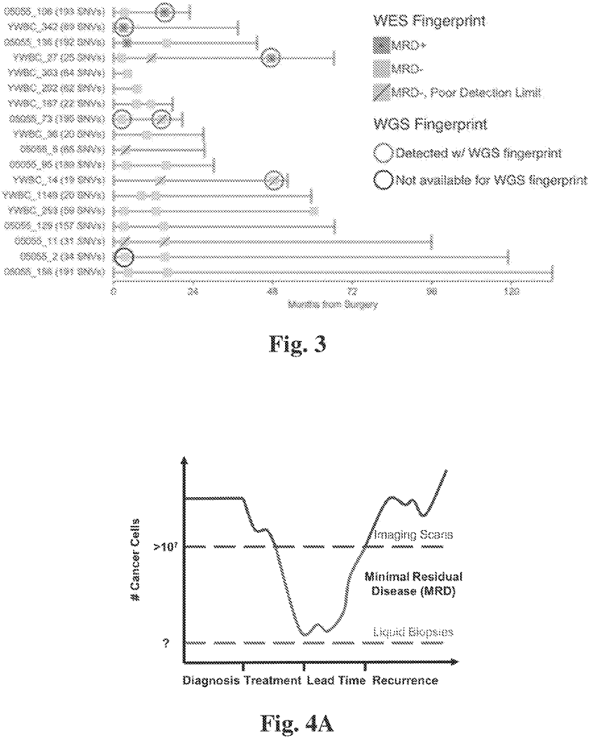

Raw VAF was calculated using the single strand consensus BAMs as consensus bases are more reliable compared to raw sequenced bases and help correct for PCR bias. Single strand consensus BAMs were used rather than the duplex BAMs as a goal was to retain the majority of sequenced reads - with duplex sequencing, more than 50% of reads can be lost due to support only being observed on one strand. For each site, a pileup was created from the single strand consensus BAM and read bases were compared to the called bases in the MAF file. Each base was categorized as reference (REF), alternate (ALT), or OTHER and the consensus family size (number of reads contributing to the consensus) was added to the site’s read counts. Raw VAF could then be calculated by comparing the number of ALT reads to the total reads (REF + ALT + OTHER) for each site. This raw VAF measurement is important for determining the efficiency of sequencing the ALT base, but may not be an accurate readout of true variant allele fraction due to PCR bias. To address this, duplex VAF has been included in FIG. 32, where duplex VAF is calculated using the consensus duplex fragments rather than family size as used in raw VAF. To assess recall, the duplex consensus BAM files were used. The consensus calling workflow gives source molecules the same family ID, so two samples from the same library have many overlapping molecules. Recall was calculated by looking at the overlap of duplex families between two samples (oftentimes a Conventional sample and a MAESTRO sample). See Supplementary FIG. 3B for an example.

### Noise Filter

Four replicate negative controls were created from the same source library via reindexing as described in DNA Extraction and Library Construction. The replicates were captured using the 10,000 SNV MAESTRO panel. For each targeted site with ALT molecules present in any of the replicates, a DSC/SSC ratio was calculated by summing all ALT supporting duplexes and dividing by the total ALT supporting single strand consensus molecules. Targets with ALT duplexes present in more than one replicate were considered “shared” whereas targets with ALT duplexes present in a single replicate were marked as “exclusive.” A single DSC/SSC ratio was chosen that maximized the number of targets shared while minimizing the number of exclusive targets.

### Probe Spike-in Experiment

MAESTRO capture was performed with a 10,000 SNV panel applied to negative control HapMap samples. Prior to post-capture PCR, ten MAESTRO probes selected randomly from the 10,000 SNV panel and synthesized by IDT were added at 1000 x concentration. This created a worstcase scenario to test the hypothesis that excess probe can create new mutant molecules by extending from real molecules, specifically during post-capture PCR (see Supplementary FIG. 5A for a schematic of this hypothesis). The usual post-PCR cleanup removed all excess probes. Second capture proceeded in the same manner.

### Tumor Fraction Estimation

Methods for calculating tumor fraction were previously described^{54,55} but some changes were made for use with MAESTRO. In a conventional sample, the full wildtype and mutant diversity is available and can inform tumor fraction. This is important as the tumor fraction methods currently rely on first calculating allele fraction (ALT depth / total depth) for all sites. In MAESTRO samples, there is often full mutant diversity, where wildtype molecules have been depleted. Because enrichment is not perfect, for each panel some targets were used that retain the full diversity of wildtype. This leverages the imperfect enrichment to estimate what the total potential depth of the sample is (how many cells likely contributed to the cfDNA library). This estimated depth is applied to all targets which allows us to calculate allele fraction (without considering copy number alterations) and subsequently tumor fraction. Supplementary FIG. 13 shows this strategy and how it compares to actual tumor fractions. These methods are not perfect in their current state, but believe that advances in quality control (i.e., testing for a handful of germline SNPs to measure unique duplexes per loci) could further improve tumor fraction estimation from enriched samples.

### Example 1 MAESTRO Uncovers the Same Mutant Duplexes With ~ 100-Fold Less Sequencing

An accurate and efficient technique to track large numbers of low abundance mutations in clinical specimens has been established (FIG. 5, top panel). The technique, called MAESTRO, utilizes allele-specific hybridization with short probes, leveraging thermodynamic differences in heteroduplex versus homoduplex DNA (FIG. 10), to enrich barcoded library molecules bearing up to 10,000 prespecified mutations. Minimal sequencing is applied, and mutations are detected on both sense strands of each DNA duplex (FIG. 5, bottom panel). MAESTRO also employs a tunable noise filter which excludes error-prone loci (Methods).

First, the maximization of fold-enrichment while minimizing loss of mutations was sought. A 1/1k dilution of sheared genomic DNA from two human cell lines was created, exclusive single nucleotide polymorphisms (SNPs) were identified as proxies for clonal mutations, and duplex sequencing libraries that were split for hybrid capture were generated. Using qPCR, it was confirmed that adapter ligation efficiencies were consistent with prior reports (Table 1), and that MAESTRO capture efficiency was only slightly lower than conventional capture (Table 2).

TABLE 1

| Input (ng) | Adjusted Library Conversion Efficiency (%) | Std Adjusted Library Conversion Efficiency (%) |
|---|---|---|
| 50 | 17.8451915 | 0.79462085 |
| 20 | 12.2496892 | 1.82621481 |
| 10 | 8.94962653 | 1.876943 |
| 3 | 7.951238 | 0.95417308 |

TABLE 2

| Protocol | Replicate | Hyb Input (ng library) | Measured molecules into capture | Measured molecules out of capture | Capture Efficiency |
|---|---|---|---|---|---|
| Conventional | 1 | 500 | 42334 | 22022 | 0.52 |
| Conventional | 2 | 500 | 42532 | 26370 | 0.62 |
| Conventional | 1 | 1500 | 142888 | 86996 | 0.61 |
| Conventional | 2 | 1500 | 127728 | 72737 | 0.57 |
| Conventional | 1 | 4000 | 343087 | 215647 | 0.63 |
| Conventional | 2 | 4000 | 368016 | 225371 | 0.61 |
| MAESTRO | 1 | 500 | 42334 | 15814 | 0.37 |
| MAESTRO | 2 | 500 | 42532 | 13646 | 0.32 |
| MAESTRO | 1 | 1500 | 142888 | 71887 | 0.50 |
| MAESTRO | 2 | 1500 | 127728 | 52686 | 0.41 |
| MAESTRO | 1 | 4000 | 321902 | 168528 | 0.52 |
| MAESTRO | 2 | 4000 | 339980 | 160004 | 0.47 |

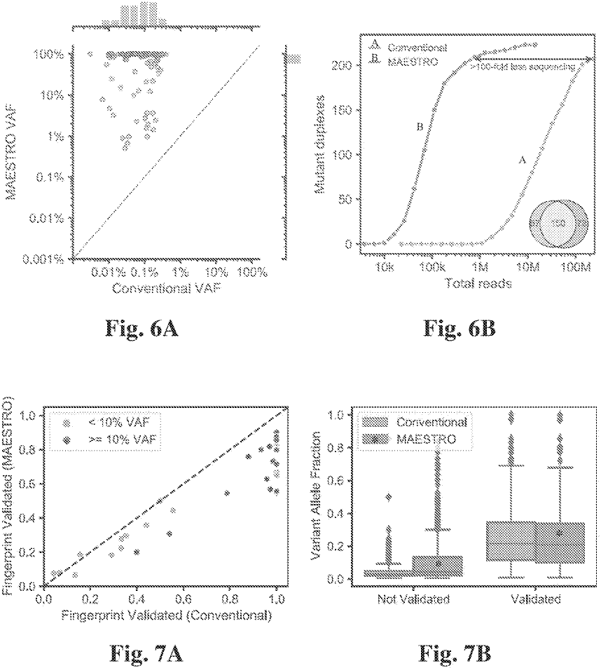

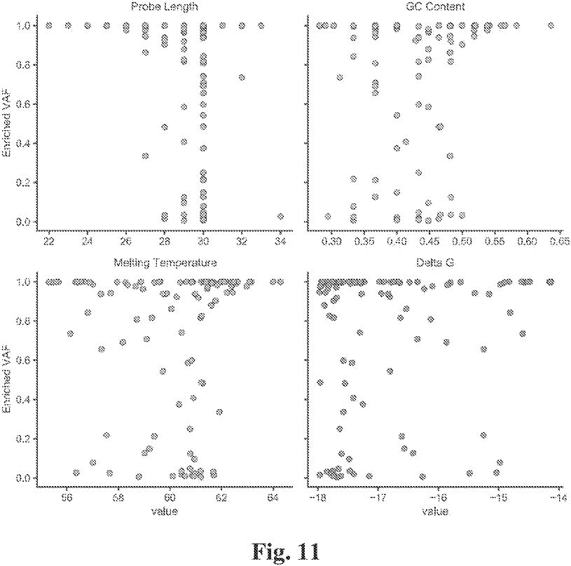

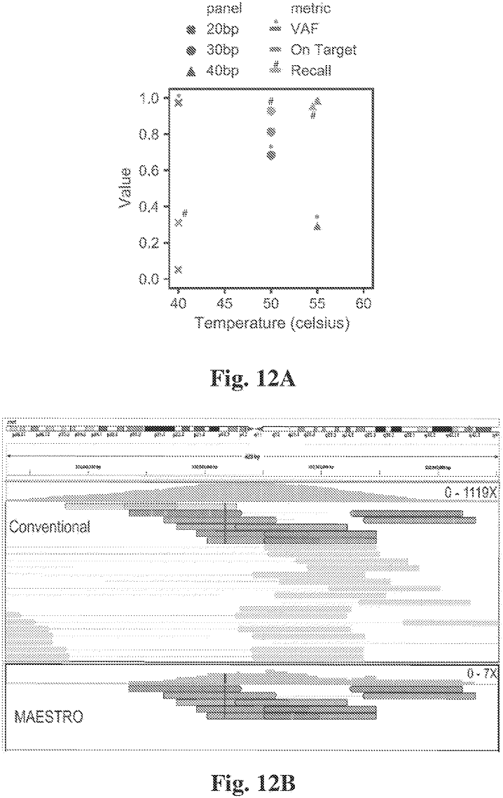

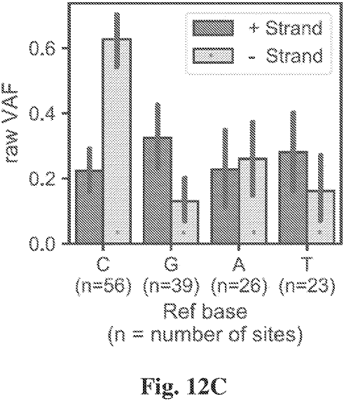

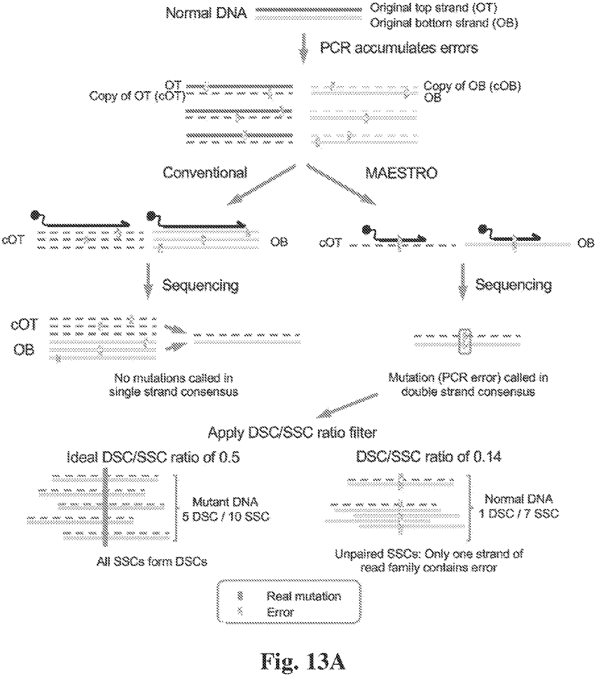

After sequencing, raw variant allele fraction (raw VAF) and recall of mutant duplexes (FIGS. 12B and 31) using MAESTRO were compared against conventional hybrid capture (120 bp probes, 65° C. annealing). By adjusting probe length and hybridization parameters, conditions (ΔG -18 to -14 kcal/mol, T=50° C., FIG. 10-12A) were established that yielded strong fold-enrichment of mutant vs. wild type alleles (median 948.3-fold, range 8.1 to 3.4E4) while uncovering the majority of mutant duplexes (FIGS. 6A-6B and 31). Indeed, the median raw VAF with MAESTRO was 0.97 (range 5.03E-3 to 1), in contrast to 6.98E- 4 (range 3.00E-5 to 3.87E-3) with Conventional. The fraction of recoverable mutations (or, enrichment ‘success rate’) was 72.5%. Interestingly, equal and opposite magnitude raw VAF changes were not observed when swapping strands of C and G reference base probes (FIG. 12C). This may be due to differences in probe characteristics (i.e. delta G, length) for each base category but further investigation is needed. MAESTRO cannot uncover more mutations than physically present in a sample; yet, by detecting each with up to 100x fewer reads, it can recover more total unique mutations, particularly when it would not otherwise be possible (e.g. due to cost) to sequence a sample to saturation.

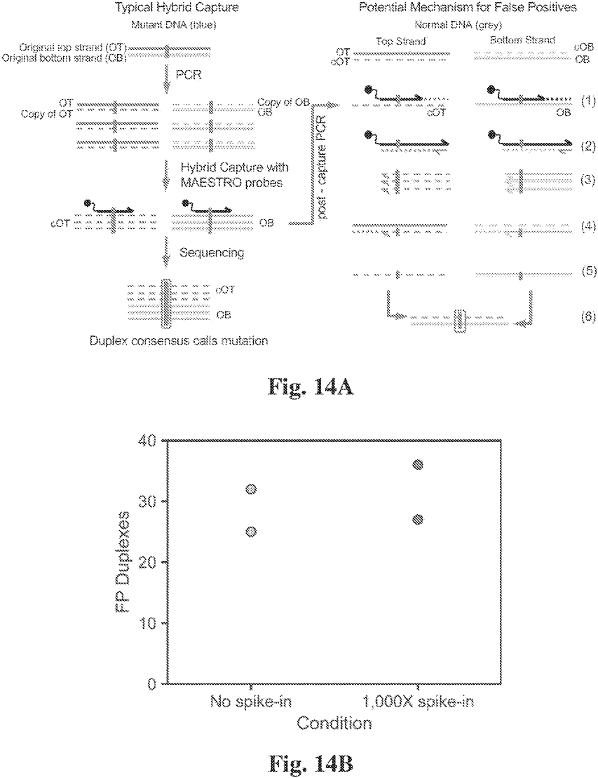

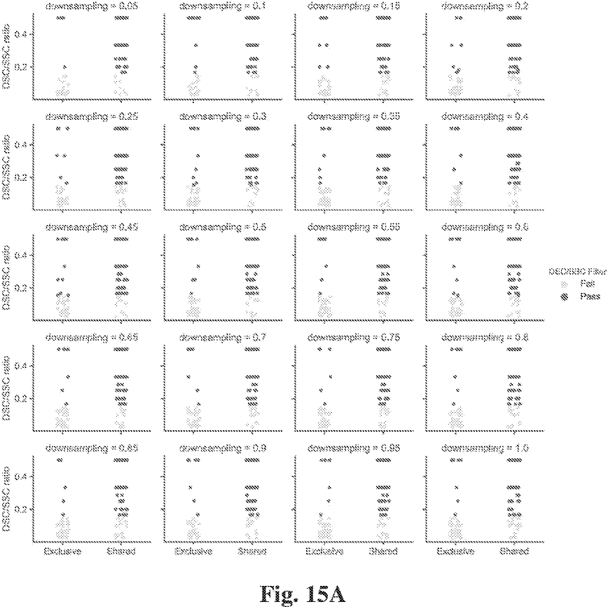

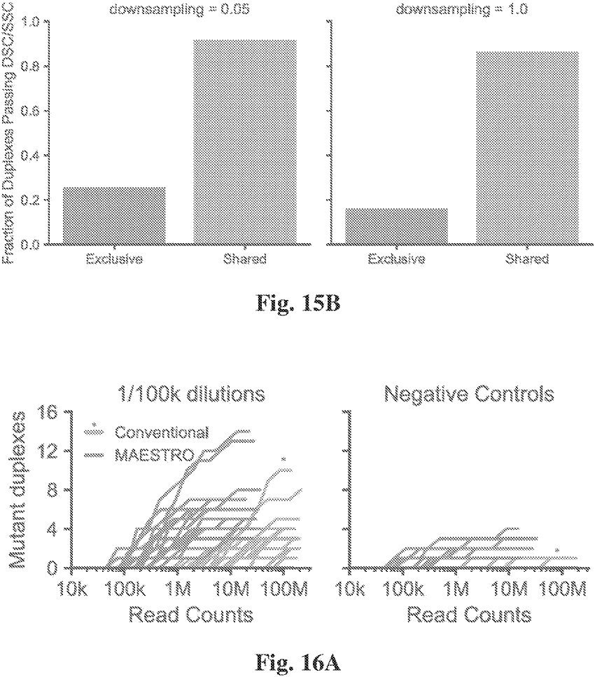

Next, the MAESTRO noise filter was tuned. This filter was designed to protect against the possibility that errors could arise independently on both strands of library molecules and, given enrichment bias, ‘collide’ to form a duplex (FIG. 13A). It works based on the assumptions that (i) errors should be impartial to read family, and (ii) error-prone loci should therefore exhibit a disproportionate number of double- (DSC) to single- (SSC) strand consensus read families bearing mutations (FIG. 13A). Sites with DSC/SSC ratios below 0.15 had poor reproducibility in replicate captures of a non-mutant library (the negative control) (FIG. 13B). The filter also protected against errors introduced by excessive PCR (FIG. 13C), and further confirmed that MAESTRO probes—which contain the mutant base-do not create false mutant duplexes (FIGS. 14A-14B). Filtering by DSC/SSC ratio was found to be robust to changes in sequencing depth with similar concordance observed at 10% of the original sequencing depth (FIGS. 15A-15B).

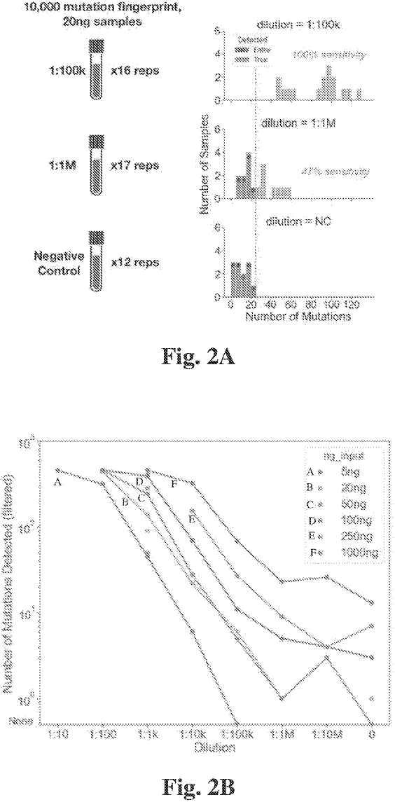

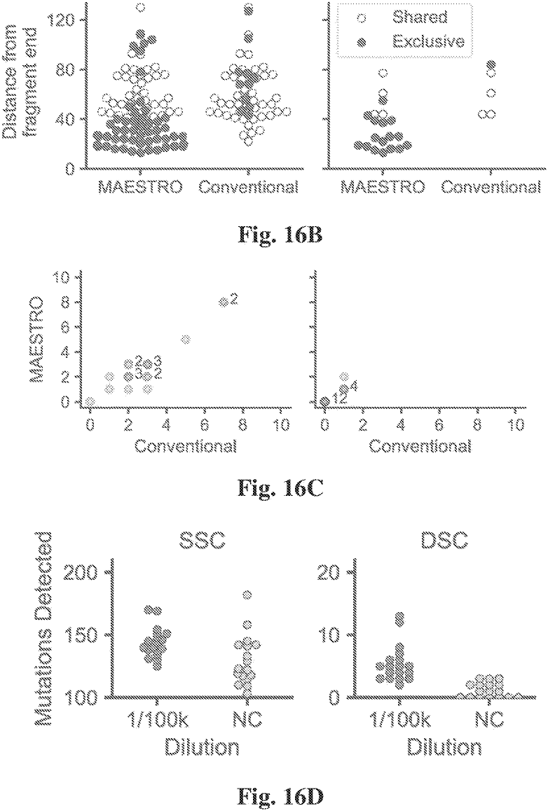

Considering the profound enrichment, it was then asked how many fewer reads would be required to detect the same mutant duplexes as Conventional. It was found that MAESTRO could uncover the majority (n=150/207) using ~100-fold less sequencing (FIG. 2B), while providing comparable specificity (FIG. 16C). Interestingly, of the 57 mutant duplexes exclusive to Conventional, 42 were detected by MAESTRO but excluded by the noise filter. These results suggest that MAESTRO can uncover the majority of mutant duplexes using significantly less sequencing.

### Example 2 MAESTRO Enables Mutation Verification From Tumor Sequencing

Expansive methods such as whole-exome and whole-genome sequencing stand to unravel the genetic basis of human diseases. However, it remains challenging to resolve low-level mutations (e.g. < 10% VAF) given insufficient depth to read each DNA molecule enough times to suppress errors. Currently, mutations discovered in sequencing studies may be orthogonally validated via technologies such as digital droplet PCR or multiplex amplicon sequencing. However, these are not highly scalable approaches and are usually restricted to a handful of mutations suspected of having potential clinical significance. It was reasoned that MAESTRO could enable rapid, low-cost verification of large numbers of mutations discovered from whole-exome and -genome sequencing. The net result would be that lower abundance mutations could be reliably discovered and verified from comprehensive sequencing studies.

To explore this, whole-exome sequencing of tumor biopsies (of varied tumor purity; median 63%, range 26 - 100%) was performed and matched with normal DNA from 16 patients. A median of 40 mutations per patient (median 40, range 13-130) were identified and both a MAESTRO and Conventional panel were created comprising all mutations for which probes could be designed. Requiring the true mutations to be detected on both strands of each duplex, similar fractions of validated mutations were found between MAESTRO and Conventional, with slightly lower fractions for MAESTRO likely due to probe dropout (FIG. 7A). Yet, the fraction of validated mutations was much higher for those which had been identified at >0.10 VAF from tumor whole-exome sequencing (median 0.75, range 0.21-0.90 for MAESTRO; median 0.98, range 0.40-1.0 for Conventional), in comparison to those which had been identified at <0.10 VAF (median 0.29, range 0.07-0.82 for MAESTRO; median 0.35, range 0.04-1.0 for Conventional, FIG. 7A). Indeed, the mutations which were found to be “not validated” tended to have the lowest VAFs from tumor whole-exome sequencing (median 0.04, range 0.01-0.83, FIG. 7B). Expectedly, higher fractions of MAESTRO-validated mutations were observed in fresh-frozen (median 0.65, range 0.62-0.77) as compared to formalin-fixed (median 0.58, range 0.10-0.76) tumor biopsies. The results suggest that MAESTRO could be an invaluable tool for validation in mutation discovery efforts.

### Example 3 MAESTRO Could Enable Liquid Biopsies to Track Up to 10,000 Individualized Mutations

To further characterize performance, and explore the feasibility to detect trace levels of ultra-rare mutations via liquid biopsy, MAESTRO was compared to conventional duplex sequencing for tracking 438 mutations in 18 x replicate 1/100k dilutions and 17 x replicate negative control samples. Sheared genomic DNA from the same two cell lines described in the previous section was used to mimic cfDNA^{8,34,38-42} and isolated 20 ng for each replicate to reflect the cfDNA in typical 10 mL blood samples. These were intended to model the scenario for which (i) a limited mass of cfDNA fragments is drawn from the bloodstream, and (ii) at sufficiently low tumor fraction such that mutations are sparsely partitioned into each blood tube. At such ‘limiting dilution’, it becomes highly unlikely that the same mutation will be drawn in replicate samples and therefore, it is necessary to track many mutations^{33,34}.

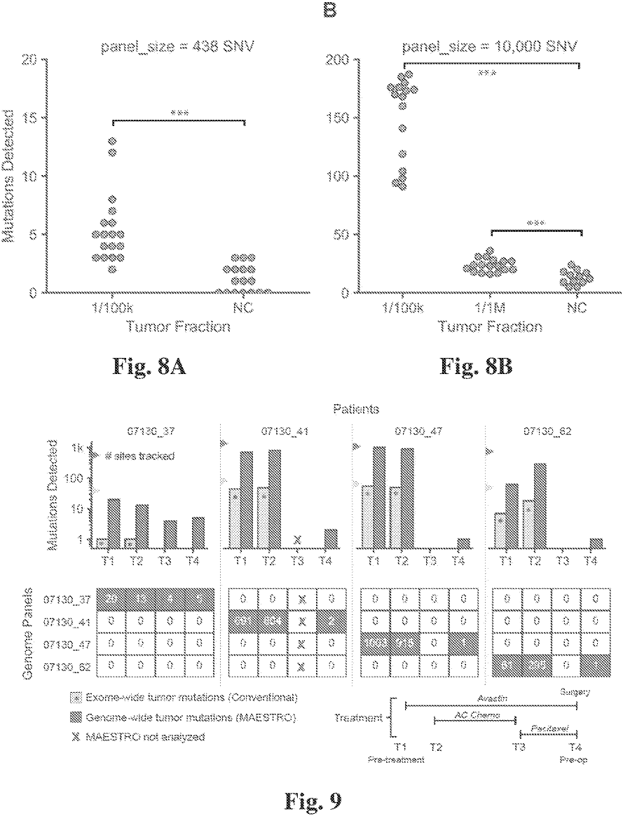

MAESTRO uncovered 81% (n=47/58) and 80% (n=⅘) of the mutant duplexes detected with Conventional across all 1/100k and negative control samples, respectively, using much less sequencing (FIG. 16A). Most that were exclusive to Conventional in the 1/100k samples (n=6/11) were detected by MAESTRO but excluded by the noise filter. MAESTRO also uncovered an additional 52 and 16 mutant duplexes across all 1/100k and negative control samples, respectively, but most were near fragment ends, which proved less likely to be captured by Conventional in these experiments (FIG. 16B). If these differences were considered, the concordance is nearly perfect (FIG. 16C). For the rest of the study the molecules that were less likely to be captured with Conventional were not removed. Importantly, MAESTRO detected significantly more mutations in the 1/100k samples than the negative controls (FIG. 8A, p=1.16E-5, Welch’s t-test). It was also confirmed that without duplex error suppression, MRD at these limiting dilutions could not have been resolved (FIG. 16D).

While MAESTRO provided comparable sensitivity and specificity using significantly less sequencing, the number of mutations detected at 1/100k dilution, of 438 tracked, was not much greater than the negative controls. Thus it was hypothesized that tracking even more mutations, e.g. 10,000—the typical number in a cancer genome⁴³—could improve the signal-to-noise ratio and enhance MRD detection. Yet, this could only be done feasibly with MAESTRO, as Conventional would require >10 billion reads (~$20,000 on the Illumina HiSeqX) to saturate duplex recovery, in contrast to about ~100 million reads (~$200) with MAESTRO, in addition to other costs of sample preparation.

Applying MAESTRO to track 10,000 mutations in 16 x replicate 1/100k dilutions, 17 x 1/1M dilutions and 12 x negative controls, a large increase in number of mutations detected in the 1/100k samples (median mutations=169, range 91 to 187) was observed, which was significantly higher than the negative controls (median 13 mutations, range 5 to 24, p=7.23E-11, FIG. 8B). Higher mutation counts were also observed in the 1/1M dilutions (median 23, range 16 to 36, p=7.47E-5), although further refinements are likely needed to enable reliable detection at 1/1M. These results suggest that tracking thousands of genome-wide mutations provides a profound boost in the signal-to-noise ratio, which is likely to be crucial for tracking MRD and guiding treatment.

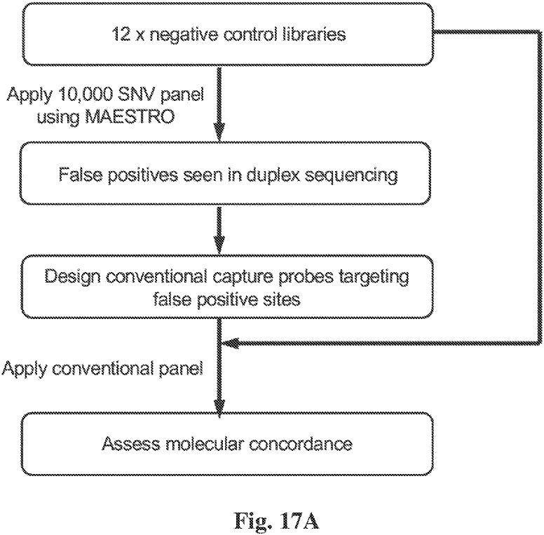

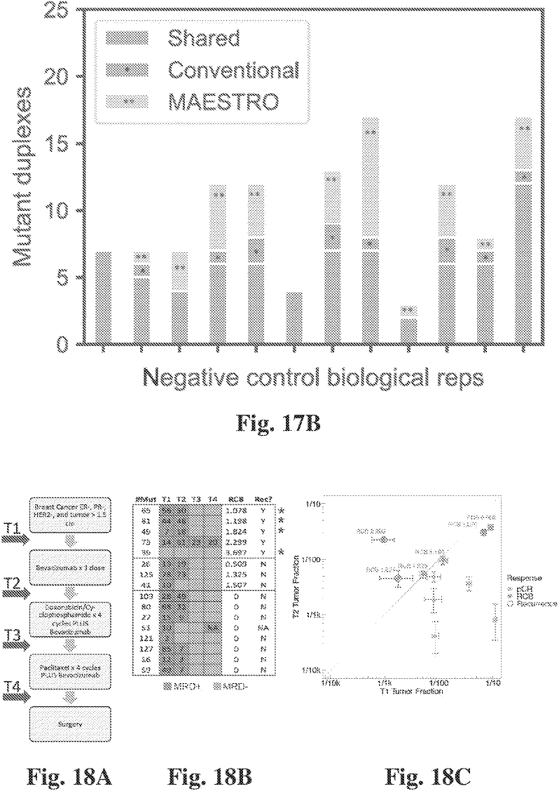

As for the mutations in the negative controls, it was reasoned that these could either be (a) true mutations that arose spontaneously with each cell division, (b) cross-contamination when cell lines were cultured, or (c) technical artifacts that have yet to be overcome in duplex sequencing. While the source cannot be discerned, the mutation counts were consistent with what was expected for scanning tens of millions of bases for potential mutation (10,000 mutations x few thousand haploid genomes of DNA) given the reported error rate of ~1x10-6 in duplex sequencing^{13,34,37}. By retesting specific loci, it was verified that the majority would have been detected with conventional duplex sequencing (FIGS. 17A-17B), suggesting that most are not artifacts of the MAESTRO protocol.

### Example 4 Tracking Thousands of Mutations From Patients’ Tumor Genomes in cfDNA Improves MRD Detection

Considering the profound boost in the signal-to-noise ratio in dilution series, whether tracking all genome-wide tumor mutations could enhance MRD detection from cfDNA was sought to be determined. For patients with some common, aggressive forms of breast cancer, standard care involves preoperative systemic chemotherapy for its utility in guiding subsequent response-based treatment^{44,45}. Patients with breast cancer enrolled in a clinical trial (16 patients) of preoperative therapy (FIG. 18A) were analyzed, with the reasoning that (i) the detectability of tumor-derived cfDNA at diagnosis could be determined, (ii) how cfDNA trends with clinical response over the course of treatment could be described, and (iii) whether preoperative MRD testing could predict the presence of residual cancer in the surgical specimen could be determined.

Reasoning that genome-wide mutation tracking would be most useful in samples with low tumor fraction, all exome-wide tumor mutations using a personalized cfDNA test built on conventional duplex sequencing³⁴ were tracked. It was found that most patients had detectable circulating tumor DNA at diagnosis (median tumor fraction 0.00858, range 0 to 0.21, Supplementary FIG. 18B) and that a decrease in tumor fraction in cfDNA between the first two time points (T1, T2) trended with clinical response (Supplementary FIG. 18C) which is consistent with prior reports²⁷⁻²⁹. Yet, MRD was detected preoperatively (T4) using conventional duplex sequencing in only one of eight patients with residual disease at the time of surgery and in only one of five who experienced future distant recurrence. The remaining four patients were chosen to explore whether genome-wide mutation tracking could enhance MRD detection.

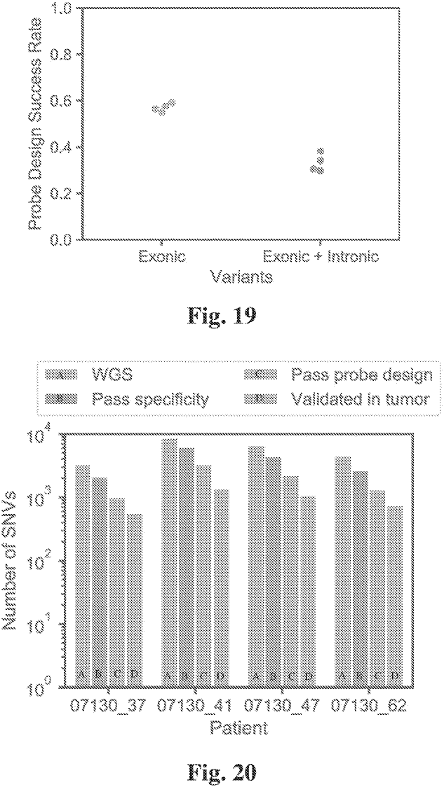

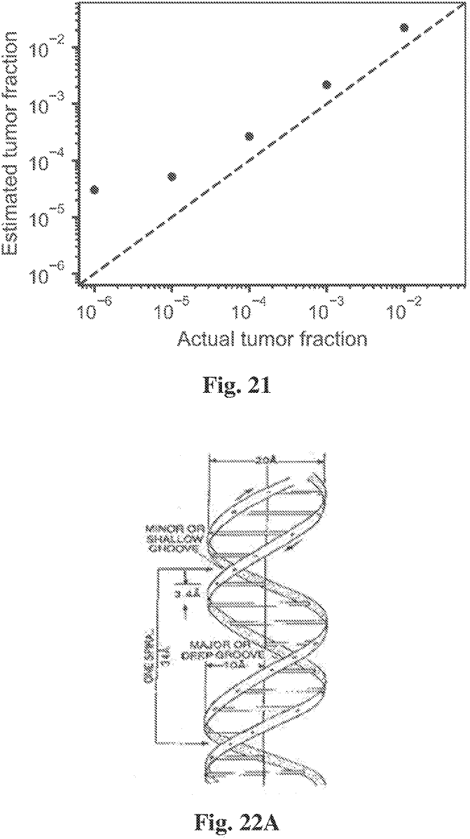

For these four patients who had tested MRD-negative preoperatively but experienced future distant recurrence, PCR-free whole-genome sequencing of their tumor biopsy specimens and blood normal DNA was performed. A median of 5575.5 (range 3385 to 8783) somatic mutations per patient was identified and, using stringent criteria for probe design, one MAESTRO test comprising 55-58% of exonic mutations and 30-38% of intronic mutations from all patients was created (FIG. 19). The MAESTRO test was applied to tumor and normal DNA and found 52% (range 41-56%) of probed mutations to be verified (FIG. 20). Then the assay was applied to all available cfDNA samples from all four patients, such that all mutations in all patients were assessed, using the unmatched samples as controls for one another. By also applying MAESTRO tests to matched germline DNA from each patient, the potential impact of variants arising from clonal hematopoiesis was limited.

It was found that tracking all tumor mutations with MAESTRO uncovered more mutations per patient in cfDNA compared to Conventional (FIG. 9, top row) and no false mutations in any unmatched samples were detected (FIG. 9, bottom row). Previous studies have shown that using > 1 mutation for MRD detection helps to protect against error^{25,33,34}. Multiple tumor mutations were uncovered preoperatively for two of the four patients, while observing profound signal enhancement in the earlier time points from all patients. These proof-of-principle results suggest that MAESTRO could enhance MRD detection by enabling all genome-wide tumor mutations to be accurately tracked in cfDNA.

### Example 5 Allele-Specific Enrichment Increases Variant Allele Fractions

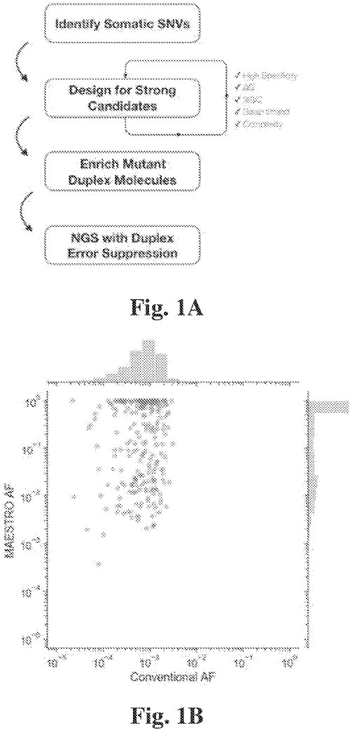

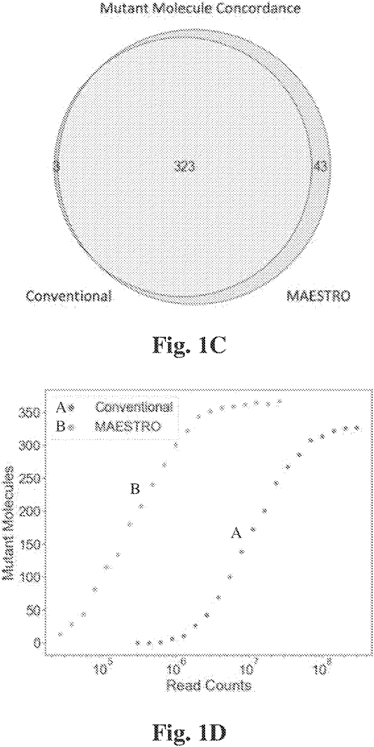

Results: Using allele-specific hybridization with short probes, leveraging thermodynamic differences in heteroduplex versus homoduplex DNA, barcoded library molecules bearing mutations identified from each patient’s tumor were enriched (FIG. 1A). As each library molecule harbors a unique molecular identifier (UMI), it was required that a mutation be observed in library molecules derived from top and bottom strand of a cfDNA duplex. To mitigate errors, it was further required the ratio of double-strand (duplex) consensus (DSC) to single-strand consensus (SSC) fragments per locus be greater than 0.3, an intrinsic measure of noise.

The potential for short, allele-specific hybrid capture probes to enrich rare mutations from a duplex sequencing library was first examined. To do this, a 20 ng, 1/1,000 dilution, sample of sheared genomic DNA from two cell lines and an amplified duplex sequencing library, were generated and split into two aliquots for hybrid capture. Four-hundred sixty-six (466) single nucleotide polymorphisms that were exclusive to the cell line in low dilution (private SNPs) were identified and developed into two sets of hybrid capture probes: conventional 120 base-pair (bp) probes against the human reference genome, and 30 bp allele-specific probes targeting the private SNPs (enrichment probes). Two rounds of hybridization were then performed followed by deep sequencing to examine the allele fractions of private SNPs. A substantial increase in variant allele fraction (VAF) was observed when comparing hybrid capture with enrichment probes (median X, range X-Y) versus conventional probes (median X, range X-Y), suggesting the potential to enrich rare mutations from a sequencing using a simple hybridization protocol (FIG. 1B).

### Example 6 Allele-Specific Enrichment Probes Require Significantly Less Sequencing

To determine whether true mutations can be resolved from errors, duplexes were formed to evaluate consensus reads and compare the molecules identified in each of the hybridization conditions. It was found that many of the same mutant duplexes, as determined by fragment start/stop position and UMI, were uncovered using conventional probes in comparison to enrichment probes (FIG. 1C). While the majority were shared in common, non-overlapping duplexes could be attributed to factors such as: a) differences in probe length relative to position of mutation in fragment; b) varied efficiency in enrichment; and/or c) low level mutations that were previously undetected, though potential errors could not be ruled out. Considering the profound boost in allele fraction afforded by hybrid capture with short probes, it was then assessed how much sequencing was required to recover those mutant duplexes. Using in silico down-sampling, it was found that significantly less sequencing was required for the enriched sampling to saturate recovery of mutant duplexes (FIG. 1D). These results suggest that short probes can uncover many of the same mutant duplexes using significantly less sequencing.

### Example 7 Allele-Specific Enrichment and Duplex Sequencing Can Improve MRD Detection

It was then assessed how MAESTRO would perform for detection of MRD in dilution series. The technique (i.e., MAESTRO) was applied to replicate 20 ng, 1:100,000 dilutions of the sheared DNA from the same cell lines. It was further assessed whether tracking of 10,000 mutations could further improve detection. More mutations were uncovered in the 1:100,000 samples (median X, range X-Y) than in the negative controls (median X, range X-Y). Tracking 10,000 mutations involves scanning up to tens of millions of fragments for potential mutation and, given an error rate of roughly 1/1,000 ,000, tens of mutations may be found in the negative controls. Nonetheless, signal in the 1/100,000 samples was well above the noise, making MRD detection readily distinguishable. Signal in the 1/1,000 ,000 samples was only slightly above background, however, and further reductions in sequencing error rate would be required to make detection at 1/1,000,000 more reliable.

To determine how this might improve MRD detection in real patient samples, MAESTRO was applied to a series of samples from patients with early stage breast cancer. Mutations had been previously tracked and identified from whole-exome sequencing and were re-analyzed using genome-wide mutations. It was found that some patients had mutations in their cfDNA that were not previously detected using smaller fingerprints, and that could now be detected, while those with previously detectable mutations had even more that could be identified. Meanwhile, simultaneous testing of negative controls confirmed high specificity. These results suggest that large fingerprint screening using mutation enrichment is feasible and may improve signal-to-noise ratio for MRD detection.

### Example 8 Minor Groove Binders Can Be Used to Improve the Specificity and Binding Properties of Allele-Specific Probes

Probe design as explained above, includes design aspect related to the Gibbs free energy (AG) of the probe at binding the target sequence containing a mutation of interest. This property of the probe increases the discrimination of the probe to the target sequence including the mutation of interest, increasing the specificity. It is envisioned that additional method for increasing this specificity can be accomplished by including additional moieties (e.g., minor groove binders (MGBs)) on the probes. Examples of MGBs are shown in FIG. 22C, which bind the minor grooves of DNA (FIGS. 22A-22B). Examples of MGBs increasing discrimination of mismatches in ODNs (Oligodeoxynucleotides) as shown in FIG. 22D. The MGBs ODNs (+MGB) are shown to have a greater free energy difference (ΔΔG) in the MGB region as compared to the ODN absent the MGB (-MGB). Additionally, the MGB are still effective at discriminating and binding target sequences at dilutions which are increasingly small (e.g., 1 copy) (FIG. 23B). Finally, MGBs are shown to increase the melting temperature (Tₘ) of bound ODN to in various configurations, Mismatches±, MGB±, wherein ODNs with no mismatches and MGBs show an elevated Tₘ (FIG. 23C). Thus, the addition of MGBs to the probes of the disclosure will improve affinity and specificity, further improving the resolution and sensitivity of the methods herein.

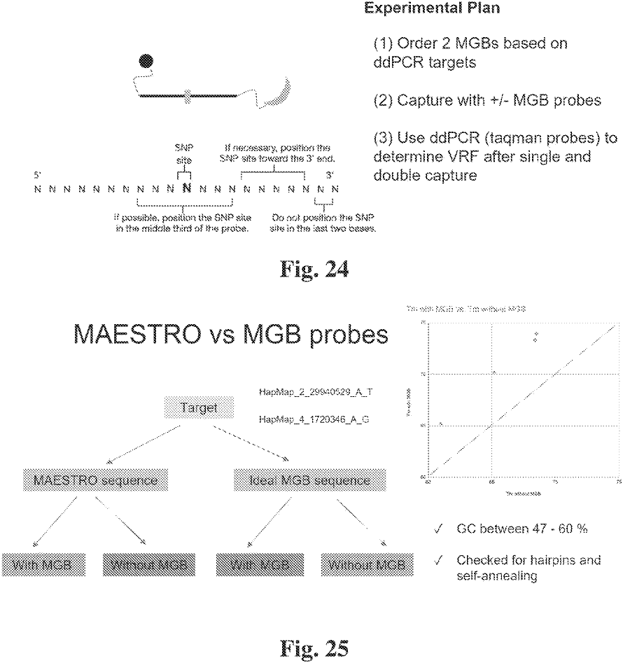

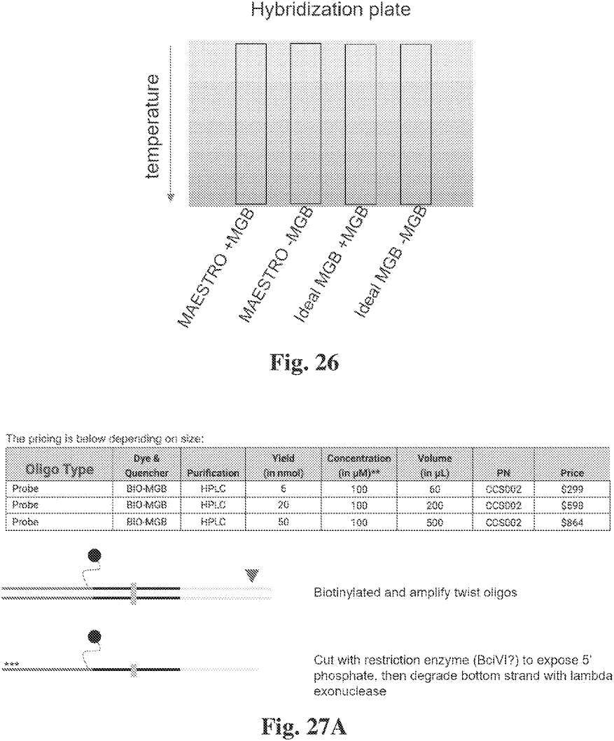

Two pairs of probes will be made, each pair consisting of a MAESTRO probe without an MBG and one with an MGB, each pair targeting one of two sequences containing a VRF (FIGS. 24-25). The probes will be biotinylated at the 5′ end of the sequence and the MGB attached to the 3′ end. The sequence of the probe will be constructed to have the SNP site in the middle third of the probe (FIG. 24). The probes will be confirmed to not comprise hairpins and contain a GC content between 47% and 60% (FIG. 25). A capture plan will utilize the four probes at 8 different temperatures to create 32 hybridization conditions. The conditions will be sampled by single and double capture for ddPCR.

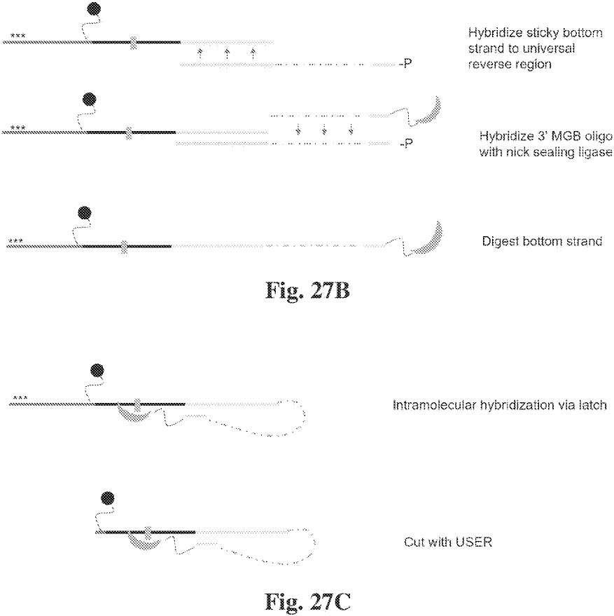

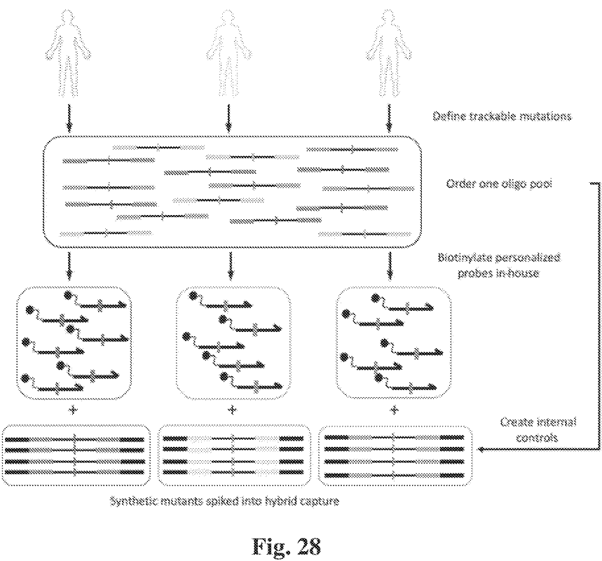

Adding MGBs to probes can be accomplished by creating the biotinylated and amplified oligos (FIG. 27A) and attaching the MGB to the 3′ end of the probe (FIG. 27B)

### Example 9 Synthetic Oligos Can Be Used to Create Internal Controls

Synthetic probes can be designed to mimic the probe target, thus creating a positive control for the allele-specific probe. Accordingly, the synthetic probes operate to provide the user of the methods feedback that the probe is binding a target sequence containing the specific mutation of interest. The probes are formulated with a fixed number of uniquely indexes per target sequences. The indexes provide the ability to track the synthetic probes and evaluate capture.

Additionally, by using a fixed number of unique indexes per target sequence, capture efficiency of the probe can be evaluated by mapping the number of unique synthetic probes captured against the specific mutations captured (FIGS. 29 and 30).

The synthetic probes comprise a central region of the probed mutation (e.g., probe target sequence), flanked by a universal forward primer on the 5′ end and a universal reverse primer on the 3′ end, which primers are flanked by sequencing adapters at the 5′ and 3′ ends (FIGS. 29-30).

### Discussion

In summary, a simple and practical approach to extend the breadth, depth, accuracy, and efficiency of mutation tracking in clinical specimens was demonstrated. This technique breaks the breadth-vs-depth ‘glass ceiling’ of DNA sequencing, enabling thousands of low-abundance mutations to be accurately tracked at low cost. This is likely to empower many types of biomedical research and diagnostic tests that demand accurate and efficient tracking of many rare mutations. For instance, it was shown that MAESTRO uniquely enables thousands of genome-wide tumor mutations to be tracked in liquid biopsies, and that this improves the detection of MRD after cancer treatment.

MAESTRO is the first method to simultaneously enrich and detect thousands of genome-wide mutations with high-accuracy sequencing. In a dilution series involving sheared genomic DNA, a median ~1000-fold enrichment from 0.1% VAF to nearly pure mutant DNA was demonstrated, which enabled the detection of most mutant duplexes using ~100-fold less sequencing. It was shown that MAESTRO could track up to 10,000 distinct, low-abundance (< 0.1% VAF) mutations scattered throughout the genome. This is important because existing methods can scan for all possible mutations within consecutive bases (e.g. within the same amplicons or probed loci) but break down when it comes to tracking many mutations in non-overlapping regions, such as genome-wide tumor mutations. MAESTRO was designed to track predefined mutations—not for mutation scanning or discovery.

This study is the first to track thousands of genome-wide tumor mutations from liquid biopsies, with sufficient breadth and depth to improve the detection of MRD. This is significant because (i) detecting MRD remains a significant unmet medical need, and (ii) while MRD detection correlates with the number of tumor mutations tracked in cfDNA^{27,34,35}, existing techniques have had limited breadth or depth. For instance, cancer gene panels typically cover just a few mutations per patient³⁷; patient-specific assays track tens to hundreds²⁷⁻³³; and whole-genome sequencing remains far too costly to apply beyond minimal depth⁴⁶. Using MAESTRO, many more mutations were detected at limiting dilutions such as 1/100k, from about 5 when 438 were tracked to almost 200 when 10,000 were tracked. Applying MAESTRO to patients undergoing neoadjuvant therapy for early-stage breast cancer, significantly more were detected when all genome-wide tumor mutations were tracked in comparison to all exome-wide mutations. With this improved sensitivity, it is believed that MAESTRO may also potentially benefit the postoperative and longitudinal detection of minimal residual disease. Bespoke genome-wide liquid biopsies reflect one potential application for MAESTRO. It was shown that tracking more mutations per patient improves the signal-to-noise ratio for MRD detection, suggesting that this could be valuable for the field. Yet, it remains to be determined whether this approach will outperform other existing tests, including epigenetic-based methods.

The profound signal enhancement that was observed for detecting MRD from liquid biopsies is likely to be important for guiding key treatment decisions such as to intensify therapy long before clinical recurrence, or to de-escalate treatment in a patient who does not have residual disease. For instance, the detection of hundreds of mutations at 1/100k limiting dilution could enable more confident determination of MRD status by placing less weight on any single mutation. This could help to overcome spurious mutations arising from clonal hematopoiesis. It could also empower new classification methods that leverage features such as fragment size that may be ‘less specific’ for any single mutation but informative when integrated across many mutations. While this approach requires whole-genome sequencing of each patient’s tumor and individualized probe design, the cost of each continues to decline, and biotinylation of oligonucleotides in-house can further help to limit costs (see Methods). It is also expected that upfront costs could be amortized over many serial MRD tests, while being offset by large savings in sequencing required per test.

MAESTRO addresses a fundamental challenge in the mutation enrichment field by using molecular barcodes to discern true mutations from low-level errors that may also be enriched. Specifically, the DSC/SSC ratio filter is a novel advance that measures intrinsic noise within each sample, but two current limitations are (i) that it needs to be tuned, and (ii) that error-prone loci are discarded, which impacts sensitivity when these regions contain real mutations. One simple way to address this is to recapture MAESTRO-detected loci with probes that target both mutant and wild type, as was done to confirm high specificity, but a better solution will be to recover all library molecules in the read family irrespective of mutant or wild type.

Another limitation of mutation enrichment is that it may lose the ability to quantify mutation abundance. To address this, internal controls may be incorporated to calibrate enrichment performance on a locus-by-locus basis, as well as incorporate probes against fixed sequences to estimate the total molecular diversity of the library and to confirm whether it was sequenced to saturation. There was also a focus on enrichment of point mutations, but it is expected that MAESTRO could also be useful for tracking other types of alterations such as insertions and deletions or structural variants. While tracking more mutations per patient could increase the number of unique cfDNA molecules sampled (and therefore, the detection limit for MRD)^{27,35,37,46}, it will never be possible to detect MRD at tumor fractions below sequencing error rates. Accordingly, the most accurate sequencing method was employed, duplex sequencing. In vitro and in silico methods exist to enrich circulating tumor DNA based upon size selection⁴⁷ and preferred end coordinates⁴⁸ but come nowhere near MAESTRO in terms of fold-enrichment.

In all, MAESTRO is a simple yet powerful approach to (i) convert low-abundance mutations into high-abundance mutations, and (ii) enable their detection with high-accuracy sequencing using significantly fewer reads. This means that it is no longer necessary to trade breadth for depth, or accuracy for efficiency, when tracking many low-abundance mutations in clinical samples. While this is expected to be useful in many ways, the ability to improve MRD detection is particularly exciting, as this could lead to more precise care for millions of cancer patients.

### References

1. Luquette, L. J., Bohrson, C. L., Sherman, M. A. & Park, P. J. Identification of somatic mutations in single cell DNA-seq using a spatial model of allelic imbalance. Nat. Commun. 10, 3908 (2019).

2. Ludwig, L. S.Lineage Tracing in Humans Enabled by Mitochondrial Mutations and Single-Cell Genomics. Cell 176, 1325-1339.e22 (2019).

3. Zahn, L. M. Mapping genotype to phenotype. Science vol. 362 555.4-556 (2018).

4. D’Gama, A. M. & Walsh, C. A. Somatic mosaicism and neurodevelopmental disease. Nat. Neurosci. 21, 1504-1514 (2018).

5. Garcia-Murillas, I. et al. Assessment of Molecular Relapse Detection in Early-Stage Breast Cancer. JAMA Oncol (2019) doi:10.1001/jamaoncol.2019.1838.

6. Canick, J. A., Palomaki, G. E., Kloza, E. M., Lambert-Messerlian, G. M. & Haddow, J. E. The impact of maternal plasma DNA fetal fraction on next generation sequencing tests for common fetal aneuploidies. Prenat. Diagn. 33, 667-674 (2013).

7. Bejar, R. et al. Somatic mutations predict poor outcome in patients with myelodysplastic syndrome after hematopoietic stem-cell transplantation. J. Clin. Oncol. 32, 2691-2698 (2014).

8. Snyder, T. M., Khush, K. K., Valantine, H. A. & Quake, S. R. Universal noninvasive detection of solid organ transplant rejection. Proc. Natl. Acad. Sci. U.S.A. 108, 6229-6234 (2011).

9. Blauwkamp, T. A. et al. Analytical and clinical validation of a microbial cell-free DNA sequencing test for infectious disease.Nat Microbiol 4, 663-674 (2019).

10. Boyd, S. D. et al. Measurement and clinical monitoring of human lymphocyte clonality by massively parallel VDJ pyrosequencing. Sci. Transl. Med. 1, 12ra23 (2009).

11. Gilbert, J. A. et al. Current understanding of the human microbiome. Nature Medicine vol. 24 392-400 (2018).

12. Lowe, A., Murray, C., Whitaker, J., Tully, G. & Gill, P. The propensity of individuals to deposit DNA and secondary transfer of low level DNA from individuals to inert surfaces. Forensic Sci. Int. 129, 25-34 (2002).

13. Schmitt, M. W. et al. Detection of ultra-rare mutations by next-generation sequencing. Proc. Natl. Acad. Sci. U.A. 109, 14508-14513 (2012).

14. Song, C. et al. Elimination of unaltered DNA in mixed clinical samples via nuclease-assisted minor-allele enrichment. Nucleic Acids Res. 44, e146 (2016).

15. Li, J. & Mike Makrigiorgos, G. COLD-PCR: a new platform for highly improved mutation detection in cancer and genetic testing. Biochemical Society Transactions vol. 37 427-432 (2009).

16. Wu, L. R., Chen, S. X., Wu, Y., Patel, A. A. & Zhang, D. Y. Multiplexed enrichment of rare DNA variants via sequence-selective and temperature-robust amplification. Nat Biomed Eng 1, 714-723 (2017).

17. Jeffreys, A. J. & May, C. A. DNA enrichment by allele-specific hybridization (DEASH): a novel method for haplotyping and for detecting low-frequency base substitutional variants and recombinant DNA molecules. Genome Res. 13, 2316-2324 (2003).

18. Gaudet, M., Fara, A.-G., Beritognolo, I. & Sabatti, M. Allele-Specific PCR in SNP Genotyping. Methods in Molecular Biology 415-424 (2009) doi:10.1007/978-1-60327-411-1_26.

19. Vargas, D. Y., Marras, S. A. E., Tyagi, S. & Kramer, F. R. Suppression of Wild-Type Amplification by Selectivity Enhancing Agents in PCR Assays that Utilize SuperSelective Primers for the Detection of Rare Somatic Mutations. J. Mol. Diagn. 20, 415-427 (2018).

20. Li, J. et al. Replacing PCR with COLD-PCR enriches variant DNA sequences and redefines the sensitivity of genetic testing. Nature Medicine vol. 14 579-584 (2008).

21. Li, J., Milbury, C. A., Li, C. & Makrigiorgos, G. M. Two-round coamplification at lower denaturation temperature-PCR (COLD-PCR)-based sanger sequencing identifies a novel spectrum of low-level mutations in lung adenocarcinoma. Hum. Mutat. 30, 1583-1590 (2009).

22. Pantel, K. & Alix-Panabières, C. Liquid biopsy and minimal residual disease - latest advances and implications for cure. Nat. Rev. Clin. Oncol. 16, 409-424 (2019).

23. Tie, J. et al. Circulating tumor DNA analysis detects minimal residual disease and predicts recurrence in patients with stage II colon cancer. Sci. Transl. Med. 8, 346ra92 (2016).

24. Chaudhuri, A. A. et al. Early Detection of Molecular Residual Disease in Localized Lung Cancer by Circulating Tumor DNA Profiling. Cancer Discov. 7, 1394-1403 (2017).

25. Coombes, R. C. et al. Personalized Detection of Circulating Tumor DNA Antedates Breast Cancer Metastatic Recurrence. Clin. Cancer Res. 25, 4255-4263 (2019).

26. Wan, J. C. M. et al. High-sensitivity monitoring of ctDNA by patient-specific sequencing panels and integration of variant reads. bioRxiv 759399 (2019) doi:10.1101/759399.

27. McDonald, B. R. et al. Personalized circulating tumor DNA analysis to detect residual disease after neoadjuvant therapy in breast cancer. Sci. Transl. Med. 11, (2019).

28. Butler, T. M. et al. Circulating tumor DNA dynamics using patient-customized assays are associated with outcome in neoadjuvantly treated breast cancer. Cold Spring Harb Mol Case Stud 5, (2019).

29. Magbanua, M. J. M. et al. Circulating tumor DNA in neoadjuvant treated breast cancer reflects response and survival. Oncology (2020) doi:10.1101/2020.02.03.20019760.

30. Moding, E. J. et al. Circulating tumor DNA dynamics predict benefit from consolidation immunotherapy in locally advanced non-small-cell lung cancer. Nature Cancer vol. 1 176-183 (2020).

31. Etienne, G. et al. Long-Term Follow-Up of the French Stop Imatinib (STIM1) Study in Patients With Chronic Myeloid Leukemia. J. Clin. Oncol. 35, 298-305 (2017).

32. Wiestner, A. Ibrutinib and Venetoclax - Doubling Down on CLL. The New England journal of medicine vol. 380 2169-2171 (2019).

33. Abbosh, C. et al. Phylogenetic ctDNA analysis depicts early-stage lung cancer evolution. Nature 545, 446-451 (2017).

34. Parsons, H. A. et al. Sensitive detection of minimal residual disease in patients treated for early-stage breast cancer. Clin. Cancer Res. (2020) doi:10.1158/1078-0432.CCR-19-3005.

35. Wan, J. C. M. et al. ctDNA monitoring using patient-specific sequencing and integration of variant reads. Sci. Transl. Med. 12, (2020).

36. Schmitt, M. W. et al. Sequencing small genomic targets with high efficiency and extreme accuracy. Nat. Methods 12,423-425 (2015).

37. Newman, A. M. et al. Integrated digital error suppression for improved detection of circulating tumor DNA. Nat. Biotechnol. 34, 547-555 (2016).

38. Newman, A. M. et al. An ultrasensitive method for quantitating circulating tumor DNA with broad patient coverage. Nat. Med. 20, 548-554 (2014).

39. Lee, H., Park, C., Na, W., Park, K. H. & Shin, S. Precision cell-free DNA extraction for liquid biopsy by integrated microfluidics. npj Precision Oncology 4, 3 (2020).

40. Mauger, F. et al. Comparison of commercially available whole-genome sequencing kits for variant detection in circulating cell-free DNA. Sci. Rep. 10, 6190 (2020).

41. Liu, D. et al. Multiplex Cell-Free DNA Reference Materials for Quality Control of Next- Generation Sequencing-Based In Vitro Diagnostic Tests of Colorectal Cancer Tolerance. Journal of Cancer vol. 9 3812-3823 (2018).

42. Tsao, D. S. et al. A novel high-throughput molecular counting method with single base-pair resolution enables accurate single-gene NIPT. Sci. Rep. 9, 14382 (2019).

43 ICGC/TCGA Pan-Cancer Analysis of Whole Genomes Consortium. Pan-cancer analysis of whole genomes. Nature 578, 82-93 (2020).

44. Masuda, N. et al. Adjuvant Capecitabine for Breast Cancer after Preoperative Chemotherapy. N. EngL J. Med. 376, 2147-2159 (2017).

45. von Minckwitz, G. et al. Trastuzumab Emtansine for Residual Invasive HER2-Positive Breast Cancer. N. Engl. J. Med. 380, 617-628 (2019).

46. Zviran, A. et al. Genome-wide cell-free DNA mutational integration enables ultrasensitive cancer monitoring. Nat. Med. 26, 1114-1124 (2020).

47. Mouliere, F. et al. Enhanced detection of circulating tumor DNA by fragment size analysis. Sci. Transl. Med. 10, (2018).

48. Jiang, P. et al. Preferred end coordinates and somatic variants as signatures of circulating tumor DNA associated with hepatocellular carcinoma. Proc. Natl. Acad. Sci. U.S.A. 115, E10925-E10933 (2018).

49. Zook, J. M. et al. An open resource for accurately benchmarking small variant and reference calls. Nat. Biotechnol. 37, 561-566 (2019).

50. 1000 Genomes Project Consortium et al. A global reference for human genetic variation. Nature 526, 68-74 (2015).

51. Zhang, D. Y. & Bae, J. H. Methods for studying nucleotide accessibility in dna and ma based on low-yield bisulfite conversion and next-generation sequencing. US Patent (2020).

52. Adalsteinsson, V. A. et al. Scalable whole-exome sequencing of cell-free DNA reveals high concordance with metastatic tumors. Nat. Commun. 8, 1324 (2017).

53. Köster, J. & Rahmann, S. Snakemake—a scalable bioinformatics workflow engine. Bioinformatics 28, 2520-2522 (2012).

54. Parsons, H. A. et al. Sensitive detection of minimal residual disease in patients treated for early-stage breast cancer. Clin. Cancer Res. (2020) doi:10.1158/1078-0432.CCR-19-3005.

55. Zhang, D. Y. & Bae, J. H. Methods for studying nucleotide accessibility in dna and rna based on low-yield bisulfite conversion and next-generation sequencing. US Patent (2020).

56. Adalsteinsson, V. A. et al. Scalable whole-exome sequencing of cell-free DNA reveals high concordance with metastatic tumors. Nat. Commun. 8, 1324 (2017).

57. Schmitt, M. W. et al. Sequencing small genomic targets with high efficiency and extreme accuracy. Nat. Methods 12,423-425 (2015).

58. Parsons, H. A. et al. Sensitive detection of minimal residual disease in patients treated for early-stage breast cancer. Clin. Cancer Res. (2020) doi:10.1158/1078-0432.CCR-19-3005.

59. Abbosh, C. et al. Phylogenetic ctDNA analysis depicts early-stage lung cancer evolution. Nature 545, 446-451 (2017).

### Other Embodiments

Embodiment 1. A method of identifying the presence of a specific mutation, comprising: (a) obtaining a pool of DNA duplexes having, suspected of having, or at risk of having the specific mutation in at least one strand, and optionally fragmenting the DNA duplexes; (b) attaching (e.g., ligating) a unique molecular identifier (UMI) to the 5′ and 3′ ends of each strand of the DNA duplexes to produce tagged duplexes, wherein the UMIs are unique to each tagged duplex; (c) amplifying the tagged duplexes by polymerase chain reactions (PCR) to produce amplified duplexes; (d) denaturing the amplified duplexes to produce single-stranded amplified DNA; (e) capturing single-stranded amplified DNA having the specific mutation using an allele-specific probe that anneals to the specific mutation to produce an enriched sample; (f) sequencing the enriched sample; and (g) confirming the presence of the specific mutation if the specific mutation is observed in both strands of the tagged duplex as identified by the UMIs.

Embodiment 2. A method comprising: (a) obtaining a pool of DNA duplexes comprising a specific mutation in at least one strand and attaching (e.g., ligating) a unique molecular identifier (UMI) to the 5′ and 3′ ends of each strand of the DNA duplexes to produce tagged duplexes, wherein the UMIs are specific to each tagged duplex; (b) amplifying the tagged duplexes by polymerase chain reactions (PCR) to produce amplified duplexes and subsequently denaturing the amplified duplexes to produce single-stranded amplified DNA; (c) capturing single-stranded amplified DNA having the specific mutation using an allele-specific probe that anneals to the specific mutation to produce an enriched sample, and sequencing the enriched sample; and (d) calculating a double-stranded consensus (DSC) to single-stranded consensus (SSC) ratio (DSC to SSC ratio) using the UMIs, and identifying the specific mutation if the DSC to SSC ratio is greater than 0.15.

Embodiment 3. The method of embodiment 1, wherein in step (e) the allele-specific probe anneals to the specific mutation at between 48 degrees Celsius (°C) and 52° C. and the probe is recovered, to produce a sample that is enriched for single-stranded amplified DNA having the specific mutation.

Embodiment 4. The method of embodiment 1 or embodiment 3, further comprising: (h) (1) calculating a double-stranded consensus (DSC) to single-stranded consensus (SSC) ratio (DSC to SSC ratio); (2) and identifying a specific mutation if the DSC to SSC ratio is greater than 0.15.

Embodiment 5. The method of embodiment 2 or embodiment 4, wherein the DSC to SSC ratio is greater than 0.2.

Embodiment 6. The method of embodiments 2 or any one of embodiments 4-5, wherein the DSC to SSC ratio is greater than 0.3.

Embodiment 7. The method any one of embodiments 1-6, wherein the allele-specific probe is about 10 to about 60 nucleotides long.

Embodiment 8. The method of any one of embodiments 1-7, wherein the allele-specific probe is about 15 to about 50 nucleotides long.

Embodiment 9. The method of any one of embodiments 1-8, wherein the allele-specific probe is about 20 to about 40 nucleotides long.

Embodiment 10. The method of any one of embodiments 1-9, wherein the allele-specific probe is about 28 to about 32 nucleotides long.

Embodiment 11. The method of any one of embodiments 1-10, wherein the allele-specific probe is 30 nucleotides long.

Embodiment 12. The method of any one of embodiments 1-11, wherein the specific mutation can be identified with at least 10 times fewer sequencing reads as compared with conventional duplex sequencing methods.

Embodiment 13. The method of any one of embodiments 1-12, wherein the specific mutation can be identified with at least 100 times fewer sequencing reads as compared with conventional duplex sequencing methods.

Embodiment 14. The method of any one of embodiments 1-13, wherein capturing of the single-stranded amplified DNA having the specific mutation using an allele-specific probe that anneals to the specific mutation is repeated on the enriched sample at least 10 times relative to a control.

Embodiment 15. The method of any one of embodiments 1-14, wherein capturing of the single-stranded amplified DNA having the specific mutation using an allele-specific probe that anneals to the specific mutation is repeated on the enriched sample at least 100 times relative to a control.

Embodiment 16. The method of any one of embodiments 1-15, wherein capturing of the single-stranded amplified DNA having the specific mutation using an allele-specific probe that anneals to the specific mutation is repeated on the enriched sample at least 1,000 times relative to a control.

Embodiment 17. The method of any one of embodiments 1-16, wherein the pool is generated from a liquid biopsy.

Embodiment 18. The method of embodiment 17, wherein the liquid biopsy is conducted on a subject or on a sample from a subject.

Embodiment 19. The method of embodiment 18, wherein the subject has a tumor, had a tumor in the past, or is suspected of having a tumor.

Embodiment 20. The method of any one of embodiments 18-19, wherein the subject has breast cancer, had breast cancer in the past, or is suspected of having breast cancer.

Embodiment 21. The method of any one of embodiments 18-20, wherein the subject is undergoing, has undergone, or will undergo, neoadjuvant therapy for early-stage breast cancer.

Embodiment 22. The method of any one of embodiments 18-21, wherein the subject is postoperative.

Embodiment 23. The method of any one of embodiments 17-22, wherein the liquid biopsy contains cell-free DNA (cfDNA).

Embodiment 24. The method of any one of embodiments 17-23, wherein the liquid biopsy is genome-wide.

Embodiment 25. The method of any one of embodiments 1-24, wherein the method is a method for detecting minimal residual disease (MRD).

Embodiment 26. The method of any one of embodiments 1-25, wherein the method is a method for detecting at least one single nucleotide polymorphism (SNP).

Embodiment 27. The method of embodiment 26, wherein at least one SNP is in the germ line.

Embodiment 28. The method of any one of embodiments 1-27, wherein the method is a method for detecting at least one insertion or deletion.

Embodiment 29. The method of any one of embodiments 1-28, wherein the method is a method for detecting at least one structural variant.

Embodiment 30. The method of any one of embodiments 1-29, wherein the pool is enriched for more than one specific mutation.

Embodiment 31. The method of any one of embodiments 1-30, wherein the pool is enriched for at least 25 specific mutations.

Embodiment 32. The method of any one of embodiments 1-31, wherein the pool is enriched for at least 50 specific mutations.

Embodiment 33. The method of any one of embodiments 1-32, wherein the pool is enriched for at least 100 specific mutations.

Embodiment 34. The method of any one of embodiments 1-33, wherein the pool is enriched for at least 500 specific mutations.

Embodiment 35. The method of any one of embodiments 1-34, wherein the pool is enriched for at least 1,000 specific mutations.

Embodiment 36. The method of any one of embodiments 1-35, wherein the method is capable of tracking up to 10,000 distinct, low-abundance specific mutations throughout the genome.

Embodiment 37. The method of embodiment 36, wherein the mutations are in non-overlapping regions of the genome.

Embodiment 38. The method of any one of embodiments 1-37, wherein the allele-specific probe is biotinylated.

Embodiment 39. The method of any one of embodiments 1-36, further comprising selecting low-noise mutations.

Embodiment 40. The method of embodiment 37, wherein the low-noise mutations comprise mutations at sites in a reference sequence comprising an adenine (A) and thymine (T) base pairing.

Embodiment 41. The method of any one of embodiments 1-40, wherein the pool includes internal controls.

Embodiment 42. The method of embodiment 41, wherein the internal controls comprise synthetic mutants that the allele-specific probes are capable of binding.

Embodiment 43. The method of embodiment 42, wherein the performance of an allele-specific probe can be assessed based on its ability to detect synthetic mutants.

Embodiment 44. The method of any one of embodiments 41-43, wherein an internal control is included for each specific mutation or duplex in the pool.

Embodiment 45. The method of any one of embodiments 1-44, wherein at least one of the allele-specific probes comprises a modification.

Embodiment 46. The method of embodiment 45, wherein the modification improves structural stability of the probe.

Embodiment 47. The method of any one of embodiments 45-46, wherein the modification improves binding affinity.

Embodiment 48. The method of any one of embodiments 1-47, wherein the allele-specific probes comprise minor groove binders (MGB).

Embodiment 49. The method of embodiment 48, wherein the MGB is attached to the 3′ end of the allele-specific probe.

Embodiment 50. The method of any one of embodiments 1-49, wherein a recovery moiety is attached to the 5′ end of the allele-specific probe.

Embodiment 51. The method of embodiment 50, wherein the recovery moiety is biotin.

Embodiment 52. A method of detecting minimal residual disease, comprising: (a) performing a liquid biopsy on a subject having, suspected of having, at risk of having, or who has previously had cancer; and (b) performing the method of any one of embodiments 1-51; wherein identification of mutations associated with tumors indicates minimal residual disease.

Embodiment 53. The method of any one of embodiments 1-52, wherein the allele-specific probe comprises a nucleotide complementary to a specific mutation, wherein the nucleotide complementary to a specific mutation is in the middle 50% of nucleotides of the allele-specific probe.

Embodiment 54. The method of any one of embodiments 1-53, wherein the allele-specific probe comprises a nucleotide complementary to a specific mutation, wherein the nucleotide complementary to a specific mutation is in the middle 34% of nucleotides of the allele-specific probe.

Embodiment 55. The method of any one of embodiments 1-54, wherein the allele-specific probe comprises a nucleotide complementary to a specific mutation, wherein the nucleotide complementary to a specific mutation is in the middle 5% of nucleotides of the allele-specific probe.

Embodiment 56. The method of any one of embodiments 1-55, wherein the Gibbs free energy (ΔG) of the allele-specific probe annealing to its complementary sequence is at least -20 kcal/mol at Temp =50° C., but no more than -12 J.

Embodiment 57. The method of any one of embodiments 1-56, wherein the Gibbs free energy (ΔG) of the allele-specific probe annealing to its complementary sequence is at least -18 kcal/mol at Temp =50° C., but no more than -14 kcal/mol at Temp =50° C.

Embodiment 58. The method of any one of embodiments 18-57, wherein the sequence of the allele-specific probe is 100% homologous with less than 10 sequences of a reference genome of the subject.

Embodiment 59. The method of any one of embodiments 18-58, wherein the sequence of the allele-specific probe is 100% homologous with less than 5 sequences of a reference genome of the subject.

Embodiment 60. A method of making an allele-specific probe, the method comprising: (a) identifying a specific mutation in a nucleic acid sequence of a genome; (b) generating a complementary nucleic acid (CNA) including a complementary base to the specific mutation; and (c) attaching a recovery moiety to the 5′ nucleotide of the allele-specific probe; wherein the complementary base is in the middle 50% of nucleotides of the CNA; wherein, the CNA comprises at least 12, but no more than 60 nucleotides; wherein the Gibbs free energy of the CNA and the nucleic acid comprising the specific mutation is at least -20, but no more than -12; wherein the annealing temperature of the allele-specific probe is at least 48° C. (°C.), but no more than 52° C.; and wherein the CNA is 100% homologous with less than 10 sequences within the genome.

Embodiment 61. An allele-specific probe according to the method of embodiment 60.

Embodiment 62. The method of embodiment 1-59, wherein the allele-specific probe is the allele-specific probe of embodiment 61.

In addition to the embodiments expressly described herein, it is to be understood that all of the features disclosed in this disclosure may be combined in any combination (e.g., permutation, combination). Each element disclosed in the disclosure may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, and can make various changes and modifications of the invention to adapt it to various usages and conditions. Thus, other embodiments are also within the claims.

### Equivalents and Scope

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists (e.g., in Markush group format), each subgroup of the elements is also disclosed, and any element(s) can be removed from the group. It should it be understood that, in general, where the disclosure, or aspects of the disclosure, is/are referred to as comprising particular elements and/or features, certain embodiments of the disclosure or aspects of the disclosure consist, or consist essentially of, such elements and/or features. For purposes of simplicity, those embodiments have not been specifically set forth in haec: verba herein. It is also noted that the terms “comprising” and “containing” are intended to be open and permits the inclusion of additional elements or steps. Where ranges are given, endpoints are included in such ranges unless otherwise specified. Furthermore, unless otherwise indicated or otherwise evident from the context and understanding of one of ordinary skill in the art, values that are expressed as ranges can assume any specific value or sub-range within the stated ranges in different embodiments of the disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the disclosure, as defined in the following claims.

What is claimed is:

1. A method of identifying the presence of a specific mutation, comprising:
(a) obtaining a pool of DNA duplexes having, suspected of having, or at risk of having the specific mutation in at least one strand, and optionally fragmenting the DNA duplexes;
(b) attaching a unique molecular identifier (UMI) to the 5′ and 3′ ends of each strand of the DNA duplexes to produce tagged duplexes, wherein the UMIs are unique to each tagged duplex;
(c) amplifying the tagged duplexes by polymerase chain reactions (PCR) to produce amplified duplexes;
(d) denaturing the amplified duplexes to produce single-stranded amplified DNA;
(e) capturing single-stranded amplified DNA having the specific mutation using an allele-specific probe that anneals to the specific mutation to produce an enriched sample;
(f) sequencing the enriched sample; and
(g) identifying the presence of the specific mutation if the specific mutation is observed in both strands of the tagged duplex as identified by the UMIs.

2. A method comprising:
(a) obtaining a pool of DNA duplexes comprising a specific mutation in at least one strand and attaching a unique molecular identifier (UMI) to the 5′ and 3′ ends of each strand of the DNA duplexes to produce tagged duplexes, wherein the UMIs are specific to each tagged duplex;
(b) amplifying the tagged duplexes by polymerase chain reactions (PCR) to produce amplified duplexes and subsequently denaturing the amplified duplexes to produce single-stranded amplified DNA;
(c) capturing single-stranded amplified DNA having the specific mutation using an allele-specific probe that anneals to the specific mutation to produce an enriched sample, and sequencing the enriched sample; and
(d) calculating a double-stranded consensus (DSC) to single-stranded consensus (SSC) ratio (DSC to SSC ratio) using the UMIs, and identifying the specific mutation if the DSC to SSC ratio is greater than 0.15.

3. The method of claim 1, wherein in step (e) the allele-specific probe anneals to the specific mutation at between 48 degrees Celsius (°C) and 52° C. and the probe is recovered, to produce a sample that is enriched for single-stranded amplified DNA having the specific mutation.

4. The method of claim 1 or claim 3, further comprising: (h)
(1) calculating a double-stranded consensus (DSC) to single-stranded consensus (SSC) ratio (DSC to SSC ratio);
(2) and identifying a specific mutation if the DSC to SSC ratio is greater than 0.15.

5. The method of claim 2 or claim 4, wherein the DSC to SSC ratio is greater than 0.2.

6. The method of claims 2, 4 or 5, wherein the DSC to SSC ratio is greater than 0.3.

7. The method any one of claims 1-6, wherein the allele-specific probe is about 10 to about 60 nucleotides long.

8. The method of any one of claims 1-7, wherein the allele-specific probe is about 15 to about 50 nucleotides long.

9. The method of any one of claims 1-8, wherein the allele-specific probe is about 20 to about 40 nucleotides long.

10. The method of any one of claims 1-9, wherein the allele-specific probe is about 28 to about 32 nucleotides long.

11. The method of any one of claims 1-10, wherein the allele-specific probe is 30 nucleotides long.

12. The method of any one of claims 1-11, wherein the specific mutation can be identified with at least 10 times fewer sequencing reads as compared with conventional duplex sequencing methods.

13. The method of any one of claims 1-12, wherein the specific mutation can be identified with at least 100 times fewer sequencing reads as compared with conventional duplex sequencing methods.

14. The method of any one of claims 1-13, wherein capturing of the single-stranded amplified DNA having the specific mutation using an allele-specific probe that anneals to the specific mutation is repeated on the enriched sample at least 10 times relative to a control.

15. The method of any one of claims 1-14, wherein capturing of the single-stranded amplified DNA having the specific mutation using an allele-specific probe that anneals to the specific mutation is repeated on the enriched sample at least 100 times relative to a control.

16. The method of any one of claims 1-15, wherein capturing of the single-stranded amplified DNA having the specific mutation using an allele-specific probe that anneals to the specific mutation is repeated on the enriched sample at least 1,000 times relative to a control.

17. The method of any one of claims 1-16, wherein the pool is generated from a liquid biopsy.

18. The method of claim 17, wherein the liquid biopsy is conducted on a subject or on a sample from a subject.

19. The method of claim 18, wherein the subject has a tumor, had a tumor in the past, or is suspected of having a tumor.

20. The method of claim 18 or 19, wherein the subject has breast cancer, had breast cancer in the past, or is suspected of having breast cancer.

21. The method of any one of claims 18-20, wherein the subject is undergoing, has undergone, or will undergo, neoadjuvant therapy for early-stage breast cancer.

22. The method of any one of claims 18-21, wherein the subject is postoperative.

23. The method of any one of claims 17-22, wherein the liquid biopsy contains cell-free DNA (cfDNA).

24. The method of any one of claims 17-23, wherein the liquid biopsy is genome-wide.

25. The method of any one of claims 1-24, wherein the method is a method for detecting minimal residual disease (MRD).

26. The method of any one of claims 1-25, wherein the method is a method for detecting at least one single nucleotide polymorphism (SNP).

27. The method of claim 26, wherein at least one SNP is in the germ line.

28. The method of any one of claims 1-27, wherein the method is a method for detecting at least one insertion or deletion.

29. The method of any one of claims 1-28, wherein the method is a method for detecting at least one structural variant.

30. The method of any one of claims 1-29, wherein step (e) further comprises using at least one additional allele-specific probe is used to capture at least one additional single-stranded amplified DNA, wherein the at least one additional allele-specific probe anneals a distinct specific mutation.

31. The method of any one of claims 1-30, wherein step (e) further comprises using at least 25 additional allele-specific probes are used to capture at least 25 additional single-stranded amplified DNA, wherein the at least 25 additional allele-specific probes anneal distinct specific mutations.

32. The method of any one of claims 1-31, wherein step (e) further comprises using at least 50 additional allele-specific probes are used to capture at least 50 additional single-stranded amplified DNA, wherein the at least 50 additional allele-specific probes anneal distinct specific mutations.

33. The method of any one of claims 1-32, wherein step (e) further comprises using at least 100 additional allele-specific probes are used to capture at least 100 additional single-stranded amplified DNA, wherein the at least 100 additional allele-specific probes anneal distinct specific mutations.

34. The method of any one of claims 1-33, wherein step (e) further comprises using at least 500 additional allele-specific probes are used to capture at least 500 additional single-stranded amplified DNA, wherein the at least 500 additional allele-specific probes anneal distinct specific mutations.

35. The method of any one of claims 1-34, wherein step (e) further comprises using at least 1,000 additional allele-specific probes are used to capture at least 1,000 additional single-stranded amplified DNA, wherein the at least 1,000 additional allele-specific probes anneal distinct specific mutations.

36. The method of any one of claims 1-35, wherein the method is capable of tracking up to 10,000 distinct, low-abundance specific mutations throughout the genome.

37. The method of claim 36, wherein the mutations are in non-overlapping regions of the genome.

38. The method of any one of claims 1-37, wherein the allele-specific probe is biotinylated.

39. The method of any one of claims 1-36, further comprising selecting low-noise mutations.

40. The method of claim 39, wherein the low-noise mutations comprise mutations at sites in a reference sequence comprising an adenine (A) and thymine (T) base pairing.

41. The method of any one of claims 1-40, wherein the pool includes internal controls.

42. The method of claim 41, wherein the internal controls comprise synthetic mutants to which the allele-specific probes are capable of binding.

43. The method of claim 42, wherein the performance of an allele-specific probe can be assessed based on its ability to detect synthetic mutants.

44. The method of any one of claims 41-43, wherein an internal control is included for each specific mutation or duplex in the pool.

45. The method of any one of claims 1-44, wherein at least one of the allele-specific probes comprises a modification.

46. The method of claim 45, wherein the modification improves structural stability of the probe.

47. The method of claim 45 or 46, wherein the modification improves binding affinity.

48. The method of any one of claims 1-47, wherein the allele-specific probes comprise a minor groove binder (MGB).

49. The method of claim 48, wherein the MGB is attached to the 3′ end of the allele-specific probe.

50. The method of any one of claims 1-49, wherein a recovery moiety is attached to the 5′ end of the allele-specific probe.

51. The method of claim 50, wherein the recovery moiety is biotin.

52. A method of detecting minimal residual disease, comprising:
(a) performing a liquid biopsy on a subject having, suspected of having, at risk of having, or who has previously had cancer; and
(b) performing the method of any one of claims 1-51; wherein identification of mutations associated with tumors indicates minimal residual disease.

53. The method of any one of claims 1-52, wherein the allele-specific probe comprises a nucleotide complementary to a specific mutation, wherein the nucleotide complementary to a specific mutation is in the middle 50% of nucleotides of the allele-specific probe.

54. The method of any one of claims 1-53, wherein the allele-specific probe comprises a nucleotide complementary to a specific mutation, wherein the nucleotide complementary to a specific mutation is in the middle 34% of nucleotides of the allele-specific probe.

55. The method of any one of claims 1-54, wherein the allele-specific probe comprises a nucleotide complementary to a specific mutation, wherein the nucleotide complementary to a specific mutation is in the middle 5% of nucleotides of the allele-specific probe.

56. The method of any one of claims 1-55, wherein the Gibbs free energy (ΔG) of the allele-specific probe annealing to its complementary sequence is at least -20 kcal/mol at Temp =50° C., but no more than -12 kcal/mol at Temp =50° C.

57. The method of any one of claims 1-56, wherein the Gibbs free energy (ΔG) of the allele-specific probe annealing to its complementary sequence is at least -18 kcal/mol at Temp =50° C., but no more than -14 kcal/mol at Temp =50° C.

58. The method of any one of claims 18-57, wherein the sequence of the allele-specific probe is 100% homologous with less than 10 sequences of a reference genome of the subject.

59. The method of any one of claims 18-58, wherein the sequence of the allele-specific probe is 100% homologous with less than 5 sequences of a reference genome of the subject.

60. A method of detecting one or more low-abundance mutations in a sample of DNA duplexes comprising:
(a) enriching the sample of DNA duplexes for the one or more low-abundance mutations, wherein the enriching step (a) comprises:
(i) optionally fragmenting the sample of DNA duplexes;
(ii) attaching a unique molecular identifier (UMI) to the top and bottom strands of each of the DNA duplexes to obtain barcoded DNA duplexes;
(iii) amplifying the barcoded DNA duplexes;
(iv) contacting the barcoded DNA duplexes with allele-specific probes specific for one or more low-abundance mutations, thereby enriching the sample of DNA for the one or more low-abundance mutations, and
(b) sequencing the enriched DNA by duplex sequencing to identify the one or more low-abundance mutations.

61. The method of claim 60, wherein the allele-specific probes specific for one or more low-abundance mutations anneals to the barcoded DNA fragments comprising the low-abundance mutations at a temperature between 48° C. and 52° C.

62. The method of any one of claims 60-61, wherein the allele-specific probes specific for one or more low-abundance mutations are about 15 to about 50 nucleotides in length.

63. The method of any one of claims 60-62, wherein the allele-specific probes specific for one or more low-abundance mutations are about 20 to about 40 nucleotides in length.

64. The method of any one of claims 60-63, wherein the allele-specific probes specific for one or more low-abundance mutations are about 28 to about 32 nucleotides in length.

65. The method of any one of claims 60-64, wherein the allele-specific probes specific for one or more low-abundance mutations are 30 nucleotides in length.

66. The method of any one of claims 60-65, wherein the step of duplex sequencing of step (b) results in single-stranded consensus (SSC) sequences of the top or bottom strand sequences and/or double-stranded consensus (DSC) sequences of the top and bottom strand sequences of the barcoded DNA fragments.

67. The method of claim 66, wherein the one or more low-abundance mutations identified in step (b) are those mutations that are present on both the top and bottom strands of the double-stranded consensus (DSC) sequences of the barcoded DNA fragments.

68. The method of any one of claims 66-67, further comprising identifying and removing those low-abundance mutations associated with those barcoded DNA fragments characterized as having a disproportionate number of double-stranded consensus (DSC) sequences to single-stranded consensus (SSC) sequences.

69. The method of any one of claims 66-68, wherein for any given barcoded DNA fragment identified as comprising a low-abundance mutation, the disproportionate number of double-stranded consensus (DSC) sequences to single-stranded consensus (SSC) sequences defines a DSC/SSC ratio.

70. The method of claim 69, wherein if the DSC/SSC ratio is below 0.15 for the any given barcoded DNA fragment, the identified low-abundance mutation is a false mutation.

71. A method of making an allele-specific probe, the method comprising:
(a) identifying a specific mutation in a nucleic acid sequence of a genome;
(b) generating a complementary nucleic acid (CNA) including a complementary base to the specific mutation; and
(c) attaching a recovery moiety to the 5′ nucleotide of the allele-specific probe;
wherein the complementary base is in the middle 50% of nucleotides of the CNA;
wherein, the CNA comprises at least 12, but no more than 60 nucleotides;
wherein the Gibbs free energy of the CNA and the nucleic acid comprising the specific mutation is at least -20, but no more than -12;
wherein the annealing temperature of the allele-specific probe is at least 48° C. (°C.), but no more than 52° C.; and
wherein the CNA is 100% homologous with less than 10 sequences within the genome.

72. An allele-specific probe produced according to the method of claim 71.

73. The method of any one of claims 1-72, wherein the allele-specific probe is the allele-specific probe of claim 71.

74. A kit, comprising, materials and/or reagents to carry out the methods of any one of claims 1-71.

75. The kit of claim 74, further comprising at least one allele-specific probe according to claim 71.

76. A kit, comprising, materials and/or reagents to carry out the method of claim 71.

77. The kit of any one of claims 74-76, further comprising a housing to carry out the methods any one of claims 1-69.

78. The kit of any one of claim 74-77, wherein the kit is capable of performing a liquid biopsy to detect one or more mutations.
